SEQUENCE LISTING (74 sequences)

SEQ ID NO: 1 - RNA, 73 nt, synthetic construct
  misc_feature 1..73: note = Synthetic: S. cerevisiae tRNALys(UUU)
  source 1..73: mol_type = other RNA
tccttgttagctcagttggtagagcgttcggcttttaaccgaaatgtcaggggttcgagccccctatgaggag

SEQ ID NO: 2 - RNA, 73 nt, synthetic construct
  misc_feature 1..73: note = Synthetic: S. cerevisiae tRNALys(CUU)
  source 1..73: mol_type = other RNA
gccttgttggcgcaatcggtagcgcgtatgactcttaatcataaggttaggggttcgagccccctacagggct

SEQ ID NO: 3 - RNA, 72 nt, synthetic construct
  misc_feature 1..72: note = Synthetic: S. cerevisiae S tRNAArg(UCU)
  source 1..72: mol_type = other RNA
gctcgcgtggcgtaatggcaacgcgtctgacttctaatcagaagattatgggttcgacccccatcgtgagtg

SEQ ID NO: 4 - RNA, 73 nt, synthetic construct
  misc_feature 1..73: note = Synthetic: Human tRNALys(UUU)
  source 1..73: mol_type = other RNA
gcccggatagctcagtcggtagagcatcagacttttaatctgagggtccagggttcaagtccctgttcgggcg

SEQ ID NO: 5 - RNA, 73 nt, synthetic construct
  misc_feature 1..73: note = Synthetic: Human tRNALys(CUU)
  source 1..73: mol_type = other RNA
gcccggctagctcagtcggtagagcatgagactcttaatctcagggtcgtgggttcgagccccacgttgggcg

SEQ ID NO: 6 - RNA, 73 nt, synthetic construct
  misc_feature 1..73: note = Synthetic: Human tRNAArg(UCU)
  source 1..73: mol_type = other RNA
ggctctgtggcgcaatggatagcgcattggacttctaattcaaaggttgtgggttcgagtcccaccagagtcg

SEQ ID NO: 7 - RNA, 155 nt, synthetic construct
  misc_feature 1..155: note = Synthetic: Targeting molecule for S cerevisiae: tRNALys(UUU)
  source 1..155: mol_type = other RNA
gggactgaatgcgctccggcgaggcagcccacatcaagtggaactacacagacttccttgtcgcaaagccgcggtcccaagagcaatccttcccgatgcgctgctcatctattaaaatcaatagtagaatgtagtaaagccgacgggagacattc

SEQ ID NO: 8 - RNA, 155 nt, synthetic construct
  misc_feature 1..155: note = Synthetic: Targeting molecule for S cerevisiae: tRNALys(CUU)
  source 1..155: mol_type = other RNA
gggactgcttatgatccggcgaggcagcccacatcaagtggaactacacagacttccttgtcgcagagtcacggtcccaagagcaatccttcccgttatgatgctcatctattaaaatcaatagtagaatgtagtagagtcatcgggagacattc

SEQ ID NO: 9 - RNA, 155 nt, synthetic construct
  misc_feature 1..155: note = Synthetic: Targeting molecule for S cerevisiae: tRNAArg(UCU)
  source 1..155: mol_type = other RNA
gggacctcttctgatacggcgagcctgcccacatcaagtggaactacacagacatgcttgtcgcgaagtcacggtcccaagagcaatccttcccgttctgatgctcatctattaaaatcaatagtagaatgtagtgaagtcagcgggagacattc

SEQ ID NO: 10 - RNA, 127 nt, synthetic construct
  misc_feature 1..127: note = Synthetic: Targeting molecule for human tRNALys(UUU)
  source 1..127: mol_type = other RNA
ccacacacctcagatccagtccagggcagcttccctgttctgcaaagtcttgtgtggacattaaaattgcgacctcagatccagtataactataaatagtaatgctgcaaagtcttcgcagacaaaa

SEQ ID NO: 11 - RNA, 127 nt, synthetic construct
  misc_feature 1..127: note = Synthetic: Targeting molecule for human tRNALys(CUU)
  source 1..127: mol_type = other RNA
ccacacacctgagatccagtccagggcagcttccctgttctgcagagtcttgtgtggacattaaaattgcgacctgagatccagtataactataaatagtaatgctgcagagtcttcgcagacaaaa

SEQ ID NO: 12 - RNA, 127 nt, synthetic construct
  misc_feature 1..127: note = Synthetic: Targeting molecule for human tRNAArg(UCU)
  source 1..127: mol_type = other RNA
ccacacactttggatacagtccagggcagcttccctgttctgcgaagtcctgtgtggacattaaaattgcctctttggatacagtataactataaatagtaatgctgcgaagtccaggcagacaaaa

SEQ ID NO: 13 - RNA, 73 nt, synthetic construct; identical to SEQ ID NO: 1
  misc_feature 1..73: note = Synthetic: Modified S. cerevisiae tRNALys(UUU)
  modified_base 36: mod_base = OTHER; note = pseudouridine
  source 1..73: mol_type = other RNA

SEQ ID NO: 14 - RNA, 73 nt, synthetic construct; identical to SEQ ID NO: 2
  misc_feature 1..73: note = Synthetic: Modified S. cerevisiae tRNALys(CUU)
  modified_base 36: mod_base = OTHER; note = pseudouridine
  source 1..73: mol_type = other RNA

SEQ ID NO: 15 - RNA, 72 nt, synthetic construct; identical to SEQ ID NO: 3
  misc_feature 1..72: note = Synthetic: Modified S. cerevisiae S tRNAArg(UCU)
  modified_base 35: mod_base = OTHER; note = pseudouridine
  source 1..72: mol_type = other RNA

SEQ ID NO: 16 - RNA, 73 nt, synthetic construct; identical to SEQ ID NO: 4
  misc_feature 1..73: note = Synthetic: Modified human tRNALys(UUU)
  modified_base 36: mod_base = OTHER; note = pseudouridine
  source 1..73: mol_type = other RNA

SEQ ID NO: 17 - RNA, 73 nt, synthetic construct; identical to SEQ ID NO: 5
  misc_feature 1..73: note = Synthetic: Modified human tRNALys(CUU)
  modified_base 36: mod_base = OTHER; note = pseudouridine
  source 1..73: mol_type = other RNA

SEQ ID NO: 18 - RNA, 73 nt, synthetic construct; identical to SEQ ID NO: 6
  misc_feature 1..73: note = Synthetic: Modified human tRNAArg(UCU)
  modified_base 36: mod_base = OTHER; note = pseudouridine
  source 1..73: mol_type = other RNA

SEQ ID NO: 19 - RNA, 127 nt, synthetic construct
  misc_feature 1..127: note = Synthetic: mRNA targeting nucleic acid molecule for pseudouridylation
  source 1..127: mol_type = other RNA
ggcacaaccttctgacctgctttctcttatgtgagtagtgttaagaactatgtgctatacaaataattgcagcaccttctgcagtataactataaatagtaatgctgcaagaactctgcagacaaaa

SEQ ID NO: 20 - DNA, 6649 bp, synthetic construct
  misc_feature 1..6649: note = Synthetic: Plasmid vector sequence for S. cerevisiae mRNA-tRNA
  source 1..6649: mol_type = other DNA
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagccgcggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtacaggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaatttcctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccacggaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagaattaattctcatgtttgacagcttatcatcggatcgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatccgatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgtcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatcctttttatttctttctaagtgggtactggcaggagtcggggcctagtttagagagaagtagactcagtcgacaagagctctttttatttctttctaagtgggtactggcaggagtcggggcctagtttagagagaagtagactcaggtaccaactcgagtttttatttctttctaagtgggtactggcaggagtcggggcctagtttagagagaagtagactcagctagcaactgcagagtaatccttcttacattgtatcgtagcgctgcatatataatgcgtaaaattttcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 21 - DNA, 5104 bp, synthetic construct
  misc_feature 1..5104: note = Synthetic: Plasmid vector sequence for human mRNA-tRNA construct
  source 1..5104: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaatactattccacttctactcaaatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaattcataaagcttgggctaagggagatgattgggtagaaagtactattctattcatttgcctcccagcctacagtcgacatagagctcttagcagcacgtaaatattggcgttaagattctaaaattatctccagtattaactgtgctgctgaaggtaccatatctagattttttttttgctcacatgttctttcctgcgttatcccctgattctgtggataaccgtattaccgccatgcat

SEQ ID NO: 22-23 - skipped (2 entries)

SEQ ID NO: 24 - DNA, 18 bp, synthetic construct
  misc_feature 1..18: note = Synthetic: sctKuuu_40-57R
  source 1..18: mol_type = other DNA
cgaacccctgacatttcg

SEQ ID NO: 25 - DNA, 18 bp, synthetic construct
  misc_feature 1..18: note = Synthetic: sctKcuu_40-57R
  source 1..18: mol_type = other DNA
cgaacccctaaccttatg

SEQ ID NO: 26 - DNA, 18 bp, synthetic construct
  misc_feature 1..18: note = Synthetic: sctRucu_39-56R
  source 1..18: mol_type = other DNA
cgaacccataatcttctg

SEQ ID NO: 27 - DNA, 21 bp, synthetic construct
  misc_feature 1..21: note = Synthetic: sctLcaa_51-71R
  source 1..21: mol_type = other DNA
agattcgaactcttgcatctt

SEQ ID NO: 28 - DNA, 19 bp, synthetic construct
  misc_feature 1..19: note = Synthetic: scSNR81_35-53R
  source 1..19: mol_type = other DNA
gtctgtgtagttccacttg

SEQ ID NO: 29 - DNA, 22 bp, synthetic construct
  misc_feature 1..22: note = Synthetic: HtKuuu_41-62R
  source 1..22: mol_type = other DNA
ggacttgaaccctggaccctca

SEQ ID NO: 30 - DNA, 22 bp, synthetic construct
  misc_feature 1..22: note = Synthetic: HtKcuu_41-62R
  source 1..22: mol_type = other DNA
gggctcgaacccacgaccctgg

SEQ ID NO: 31 - DNA, 20 bp, synthetic construct
  misc_feature 1..20: note = Synthetic: HtSgcu_47-66R
  source 1..20: mol_type = other DNA
cgaacccacgcgtgcagagc

SEQ ID NO: 32 - RNA, 19 nt, synthetic construct
  misc_feature 1..19: note = Synthetic: sctKuuu-U36rH
  modified_base 1..5: mod_base = OTHER; note = 2'-O-methylation
  modified_base 6..9: mod_base = OTHER; note = deoxyribonucleic
  modified_base 10..19: mod_base = OTHER; note = 2'-O-methylation
  source 1..19: mol_type = other RNA
ggttaaaagccgaatgctc

SEQ ID NO: 33 - RNA, 19 nt, synthetic construct
  misc_feature 1..19: note = Synthetic: sctKcuu-U36rH
  modified_base 1..5: mod_base = OTHER; note = 2'-O-methylation
  modified_base 6..9: mod_base = OTHER; note = deoxyribonucleic
  modified_base 10..19: mod_base = OTHER; note = 2'-O-methylation
  source 1..19: mol_type = other RNA
gattaagagtcatatgcgc

SEQ ID NO: 34 - RNA, 19 nt, synthetic construct
  misc_feature 1..19: note = Synthetic: sctRucu-U36rH
  modified_base 1..5: mod_base = OTHER; note = 2'-O-methylation
  modified_base 6..9: mod_base = OTHER; note = deoxyribonucleic
  modified_base 10..19: mod_base = OTHER; note = 2'-O-methylation
  source 1..19: mol_type = other RNA
gattagaagtcagatgcgt

SEQ ID NO: 35 - DNA, 37 bp, synthetic construct
  misc_feature 1..37: note = Synthetic: scCUP1K30UAA_10-23
  source 1..37: mol_type = other DNA
ctacatgattaggctagctacaacgatggcattgttc

SEQ ID NO: 36 - DNA, 35 bp, synthetic construct
  misc_feature 1..35: note = Synthetic: scCUP1stop_10-23
  source 1..35: mol_type = other DNA
agtcgacttaggctagctacaacgattcccagagc

SEQ ID NO: 37 - DNA, 18 bp, synthetic construct
  misc_feature 1..18: note = Synthetic: Oligo_dT-18
  source 1..18: mol_type = other DNA
tttttttttttttttttt

SEQ ID NO: 38 - DNA, 20 bp, synthetic construct
  misc_feature 1..20: note = Synthetic: CUP1_50-69F
  source 1..20: mol_type = other DNA
gccaatgtggtagctgcaaa

SEQ ID NO: 39 - DNA, 20 bp, synthetic construct
  misc_feature 1..20: note = Synthetic: CUP1_133-152R
  source 1..20: mol_type = other DNA
tcagacttgttaccgcaggg

SEQ ID NO: 40 - DNA, 20 bp, synthetic construct
  misc_feature 1..20: note = Synthetic: mCherry_201-220F
  source 1..20: mol_type = other DNA
ccctcagttcatgtacggct

SEQ ID NO: 41 - DNA, 20 bp, synthetic construct
  misc_feature 1..20: note = Synthetic: mCherry_302-321R
  source 1..20: mol_type = other DNA
gccgtcctcgaagttcatca

SEQ ID NO: 42 - DNA, 20 bp, synthetic construct
  misc_feature 1..20: note = Synthetic: EGFP_466-485F
  source 1..20: mol_type = other DNA
aagcagaagaacggcatcaa

SEQ ID NO: 43 - DNA, 20 bp, synthetic construct
  misc_feature 1..20: note = Synthetic: EGFP_542-561R
  source 1..20: mol_type = other DNA
gggggtgttctgctggtagt

SEQ ID NO: 44 - DNA, 20 bp, synthetic construct
  misc_feature 1..20: note = Synthetic: TDH3_151-170F
  source 1..20: mol_type = other DNA
cacggtagatacgctggtga

SEQ ID NO: 45 - DNA, 20 bp, synthetic construct
  misc_feature 1..20: note = Synthetic: TDH3_254-273R
  source 1..20: mol_type = other DNA
gtcaacgttggaagaacccc

SEQ ID NO: 46 - DNA, 22 bp, synthetic construct
  misc_feature 1..22: note = Synthetic: hACTB_744-765F
  source 1..22: mol_type = other DNA
caccattggcaatgagcggttc

SEQ ID NO: 47 - DNA, 22 bp, synthetic construct
  misc_feature 1..22: note = Synthetic: hACTB_941-962R
  source 1..22: mol_type = other DNA
gccagggcagtgatctccttct

SEQ ID NO: 48 - RNA, 14 nt, synthetic construct
  misc_feature 1..14: note = Synthetic: Cup1K30UAAmRNA
  modified_base 7: mod_base = OTHER; note = pseudouridine
  source 1..14: mol_type = other RNA
tgccaataatcatg

SEQ ID NO: 49 - RNA, 14 nt, synthetic construct
  misc_feature 1..14: note = Synthetic: Cup1K30UGAmRNA
  modified_base 7: mod_base = OTHER; note = pseudouridine
  source 1..14: mol_type = other RNA
tgccaatgatcatg

SEQ ID NO: 50 - RNA, 14 nt, synthetic construct
  misc_feature 1..14: note = Synthetic: Cup1K30UAGmRNA
  modified_base 7: mod_base = OTHER; note = pseudouridine
  source 1..14: mol_type = other RNA
tgccaatagtcatg

SEQ ID NO: 51 - RNA, 27 nt, synthetic construct
  misc_feature 1..27: note = Synthetic: PTC(K30X)-gRNA
  stem_loop 8..9
  variation 17..24: note = n is a, c, g, or u
  source 1..27: mol_type = other RNA
catgatacgtttggcannnnnnnnata

SEQ ID NO: 52 - RNA, 17 nt, synthetic construct
  misc_feature 1..17: note = Synthetic: pre-tRNALys(UUU)
  modified_base 9: mod_base = OTHER; note = pseudouridine
  source 1..17: mol_type = other RNA
tcggcttttaagcgyat

SEQ ID NO: 53 - RNA, 17 nt, synthetic construct
  misc_feature 1..17: note = Synthetic: tRNALys(CUU)
  modified_base 9: mod_base = OTHER; note = pseudouridine
  source 1..17: mol_type = other RNA
atgactcttaatcataa

SEQ ID NO: 54 - RNA, 17 nt, synthetic construct
  misc_feature 1..17: note = Synthetic: tRNAArg(UCU)
  modified_base 9: mod_base = OTHER; note = pseudouridine
  source 1..17: mol_type = other RNA
ctgacttctaatcagaa

SEQ ID NO: 55 - RNA, 28 nt, synthetic construct
  misc_feature 1..28: note = Synthetic: Sc-TKu
  stem_loop 9..10
  variation 20..25: note = n is a, c, g, or u
  source 1..28: mol_type = other RNA
atgcgctgcgtaaagccgannnnnnaca

SEQ ID NO: 56 - RNA, 28 nt, synthetic construct
  misc_feature 1..28: note = Synthetic: Sc-TKc
  stem_loop 9..10
  variation 20..25: note = n is a, c, g, or u
  source 1..28: mol_type = other RNA
ttatgatgcgtagagtcatnnnnnnaca

SEQ ID NO: 57 - RNA, 28 nt, synthetic construct
  misc_feature 1..28: note = Synthetic: Sc-TR
  stem_loop 9..10
  variation 20..25: note = n is a, c, g, or u
  source 1..28: mol_type = other RNA
ttctgatgcgtgaagtcagnnnnnnaca

SEQ ID NO: 58 - DNA, 22 bp, synthetic construct
  misc_feature 1..22: note = Synthetic: DNAzyme 10-23
  source 1..22: mol_type = other DNA
ttaggctagctacaacgatggy

SEQ ID NO: 59 - RNA, 18 nt, synthetic construct
  misc_feature 1..18: note = Synthetic: mCherryK14uAA mRNA
  modified_base 9: mod_base = OTHER; note = pseudouridine
  source 1..18: mol_type = other RNA
ccatcatctaagagttca

SEQ ID NO: 60 - RNA, 18 nt, synthetic construct
  misc_feature 1..18: note = Synthetic: mCherryK14uAG mRNA
  modified_base 9: mod_base = OTHER; note = pseudouridine
  source 1..18: mol_type = other RNA
ccatcatctaggagttca

SEQ ID NO: 61 - RNA, 17 nt, synthetic construct
  misc_feature 1..17: note = Synthetic: tRNALys(UUU)
  modified_base 8: mod_base = OTHER; note = pseudouridine
  source 1..17: mol_type = other RNA
agacttttaatctgagg

SEQ ID NO: 62 - RNA, 17 nt, synthetic construct
  misc_feature 1..17: note = Synthetic: tRNALyS(CUU)
  modified_base 8: mod_base = OTHER; note = pseudouridine
  source 1..17: mol_type = other RNA
rgactcttaatcycagg

SEQ ID NO: 63 - RNA, 29 nt, synthetic construct
  misc_feature 1..29: note = Synthetic: Hs-PTC(K14x)_gRNA
  stem_loop 10..11
  variation 21..26: note = n is a, c, g, or u
  source 1..29: mol_type = other RNA
tgaactctagctgatgatggnnnnnnaca

SEQ ID NO: 64 - RNA, 29 nt, synthetic construct
  misc_feature 1..29: note = Synthetic: Hs-TKu
  stem_loop 10..11
  variation 20..26: note = n is a, c, g, or u
  source 1..29: mol_type = other RNA
cctcagattcgaaaagtctnnnnnnnaca

SEQ ID NO: 65 - RNA, 29 nt, synthetic construct
  misc_feature 1..29: note = Synthetic: Hs-TKc
  stem_loop 10..11
  variation 20..26: note = n is a, c, g, or u
  source 1..29: mol_type = other RNA
cctgrgattcgaagagtctnnnnnnnaca

SEQ ID NO: 66-68 - skipped (3 entries)

SEQ ID NO: 69 - RNA, 15 nt, synthetic construct
  misc_feature 1..15: note = Synthetic: PTC(K30X) mRNA
  variation 7..9: note = (uaa) or (uag) or (uga)
  source 1..15: mol_type = other RNA
tgccaannntcatgt

SEQ ID NO: 70 - RNA, 15 nt, synthetic construct
  misc_feature 1..15: note = Synthetic: PTC(K30X) pseudouridylated mRNA
  variation 7..9: note = (uaa) or (uag) or (uga) where u is pseudouridine
  source 1..15: mol_type = other RNA
tgccaannntcatgt

SEQ ID NO: 71 - RNA, 15 nt, synthetic construct
  misc_feature 1..15: note = Synthetic: PTC(K14X) mRNA
  variation 7..9: note = (uaa) or (uag) or (uga)
  source 1..15: mol_type = other RNA
atcatcnnngagttc

SEQ ID NO: 72 - RNA, 15 nt, synthetic construct
  misc_feature 1..15: note = Synthetic: PTC(K14X) pseudouridylated mRNA
  variation 7..9: note = (uaa) or (uag) or (uga) where u is pseudouridine
  source 1..15: mol_type = other RNA
atcatcnnngagttc

SEQ ID NO: 73 - protein, 8 aa, synthetic construct
  REGION 1..8: note = Synthetic: Tag monoclonal antibody
DYKDDDDK

SEQ ID NO: 74 - DNA, 21 bp, synthetic construct
  misc_feature 1..21: note = Synthetic: non-targeting shRNA
  source 1..21: mol_type = other DNA
caacaagatgaagagcaccaa

(19) United States
(12) Patent Application Publication
(10) Pub. No.: US 2026/0265742 A1
(43) Pub. Date: Sep. 10, 2026

(54) A METHOD OF TREATING A PREMATURE TRANSLATION TERMINATION CODON N1N2N3 (PTC)-RELATED DISEASE BY NONSENSE SUPPRESSION IN A SUBJECT

(71) Applicant: UNIVERSITY OF ROCHESTER, Rochester, NY (US)

(72) Inventors: Yi-Tao YU, Pittsford, NY (US); Yi Pan, Rochester, NY (US)

(21) Appl. No.: 19/167,151

(22) PCT Filed: Apr. 18, 2024
(86) PCT No.: PCT/US24/25260

§ 371 Date: Sep. 19, 2025

Related U.S. Application Data
(60) Provisional application No. 63/498,957, filed on Apr. 28, 2023.

(51) Int. Cl. C12N 15/113

(52) U.S. Cl. CPC C12N 15/113; C12N 2310/315; C12N 2310/321; C12N 2310/335

(57) ABSTRACT

Compositions and methods for treating a premature translation termination codon N1N2N3 (PTC)-related disease by nonsense suppression in a subject is disclosed. The method comprises the steps of: a) modifying a PTC in an mRNA in a cell of the subject by converting a uridine (U) residue in the PTC into a pseudouridine (Ψ) to create a modified mRNA, wherein the PTC results in the PTC-related disease, wherein the uridine is at position 1 (N1) in the PTC; b) providing a modified tRNA in the cell, wherein the modified tRNA comprises a modified uridine at position N36 of an anticodon sequence (N34N35N36); wherein the modified uridine in the modified tRNA is capable of paring with the pseudouridine (Ψ) in the modified mRNA to attach an amino acid to the translation of the modified mRNA and produce a full length translation product from the modified mRNA. Also disclosed is a method for changing coding specificity using Ψ-Ψ pair between tRNA and mRNA.

This application is a national stage application of International Application No. PCT/US2024/025260, filed Apr. 18, 2024, which claims priority from U.S. Provisional Application No. 63/498,957, filed Apr. 28, 2023. The entirety of all the aforementioned applications is incorporated herein by reference.

This invention was made with government support under GM138387 and CA241111 awarded by the National Institute for Health. The government has certain rights in the invention.

### FIELD

This application relates generally to the field of medicine, and in particular, to the control of gene expression, nonsense suppression, and modification of coding specificity.

### BACKGROUND

A nonsense suppressor is a factor that can inhibit the effect of a nonsense mutation. Nonsense suppressors can be generally divided into two classes: (1) a mutated tRNA which can bind with a termination codon on mRNA; 2) a mutation on ribosomes decreasing the effect of a termination codon.

Nonsense suppressors are useful molecular biology tools, but can also result in problematic side effects, since all identical stop codons in the genome will also be suppressed to the same degree. In synthetic biology, artificial suppressor elongator tRNAs are used to incorporate unnatural amino acids at nonsense codons placed in the coding sequence of a gene.

There remains a need for nonsense suppression that can more effectively treat premature termination codon-related diseases without causing undesirable side effects.

### SUMMARY

An aspect of the application is an isolated modified tRNA, comprising a modified anticodon sequence N34N35N36 that comprises a modified uridine at position 36 (N36) of the anticodon sequence.

An aspect of the present application relates a method of treating a premature translation termination codon N1N2N3 (PTC)-related disease by nonsense suppression in a subject, the method comprising the steps of: a) modifying a PTC in an mRNA in a cell of the subject by converting a uridine (U) residue in the PTC into a pseudouridine (Ψ) to create a modified mRNA, wherein the PTC results in the PTC-related disease, wherein the uridine is at position 1 (N1) in the PTC; b) providing a modified tRNA in the cell, wherein the modified tRNA comprises a modified uridine at position N36 of an anticodon sequence (N34, N35, N36); wherein the modified uridine in the modified tRNA is capable of pairing with the pseudouridine (Ψ) in the modified mRNA to attach an amino acid to the translation of the modified mRNA and produce a full length translation product from the modified mRNA.

An aspect of the application relates to a method of reading through a premature translation termination codon N1N2N3 (PTC) in an mRNA comprising: a) modifying the PTC in the mRNA in a cell by converting a uridine (U) residue in the PTC into a pseudouridine (Ψ) to create a modified mRNA, wherein the uridine is at position 1 (N1) in the PTC; b) providing a modified tRNA in the cell, wherein the modified tRNA comprises a modified uridine at the last position (position 36 or N36) of an anticodon sequence (N34N35N36) of the modified tRNA; wherein the modified uridine in the modified tRNA is capable of paring with the pseudouridine (Ψ) in the modified mRNA to incorporate an amino acid at the modified PTC and produce a full length translation product from the modified mRNA.

Another aspect of the application relates to a method of expanding a genetic code based on Ψ-Ψ pair decoding. The method comprises the step of a) modifying an original genetic codon (N1N2N3) that contains one or more uridines (U) in a mRNA in a cell by converting the one or more U residue(s) in the genetic codon into pseudouridine (Ψ) to create a modified genetic codon; b) providing a modified tRNA in the cell, wherein the modified tRNA comprises an anticodon that (1) contains one or more pseudouridines (Ψ) that pair with the one or more pseudouridines (Ψ) in the modified genetic codon, and (2) complements to the modified genetic codon at positions other than the one or more pseudouridines (Ψ), wherein the modified tRNA is capable of pairing with the modified genetic codon in the mRNA and incorporates an amino acid that is different from the amino acid encoded by the original genetic codon into a peptide encoded by the mRNA.

Another aspect of the application relates to a mRNA/tRNA complex, comprising: a modified mRNA comprising a modified premature translation termination codon N1N2N3 (PTC), wherein the uridine at position N1 of the PTC has been converted to a pseudouridine (Ψ); and a modified tRNA, wherein the tRNA comprises a modified uridine at position N36 of the anticodon sequence N34N35N36 of the tRNA, wherein the pseudouridine (Ψ) in the modified mRNA pairs with the modified uridine in the anticodon sequence of the modified tRNA. Another aspect of the application is a kit for treating a PTC-related disease by nonsense suppression, the kit comprising: (1) a modified tRNA comprising a modified uridine at position 36 (N36) of an anticodon sequence N34N35N36 of the tRNA; and (2) a nucleic acid molecule for pseudouridylation of a target uridine in a target mRNA in a cell, wherein the nucleic acid molecule comprises a guide region capable of forming a partially double stranded nucleic acid complex with the target mRNA comprising the target uridine, wherein the partially double stranded nucleic acid complex is capable of engaging a mammalian pseudouridylation enzyme, wherein the guide region assists in positioning the target uridine in the partially double stranded nucleic acid complex for it to be converted to a pseudouridine by the mammalian pseudouridylation enzyme, wherein the target uridine is a uridine in a PTC; (3) instructions for using the kit.

Another aspect of the present application relates to a kit for treating a PTC-related disease by nonsense suppression, the kit comprising: (1) a tRNA targeting nucleic acid molecule for pseudouridylation of a target uridine in a target tRNA, wherein the tRNA targeting nucleic acid molecule comprises a guide region capable of forming a partially double stranded nucleic acid complex with the target tRNA in a cell, wherein the partially double stranded nucleic acid complex is capable of engaging a mammalian pseudouridylation enzyme, wherein the guide region assists in positioning the uridine in the tRNA in the partially double stranded nucleic acid complex for it to be converted to a pseudouridine by the mammalian pseudouridylation enzyme; and (2) a nucleic acid molecule for pseudouridylation of a target uridine in a target mRNA in a cell, wherein the nucleic acid molecule comprises a guide region capable of forming a partially double stranded nucleic acid complex with the target mRNA comprising the target uridine, wherein the partially double stranded nucleic acid complex is capable of engaging a mammalian pseudouridylation enzyme, wherein the guide region assists in positioning the target uridine in the partially double stranded nucleic acid complex for it to be converted to a pseudouridine by the mammalian pseudouridylation enzyme, wherein the target uridine is a uridine in a PTC; (3) instructions for using the kit.

### BRIEF DESCRIPTION OF THE DRAWINGS

FIG. 1 shows the two-dimensional structure of a tRNA molecule

FIG. 2 shows the uridine and the pseudouridine (Ψ) structure. The hydrogen bond acceptor (a) and hydrogen bond donor (d) sites are shown.

FIG. 3 shows proposed U-U, Ψ-U, and Ψ-Ψ base-pairing interactions. There are two possible structures where two hydrogen bonds are formed between U and U (U-U base pair); there are three possible structures where two hydrogen bonds are formed between Ψ and U (Ψ-U base pair); there are four possible structures where two hydrogen bonds are formed between Ψ and Ψ (Ψ-Ψ base pair).

FIG. 4 shows a generic designed molecule and generic substrate RNA. The targeted uridine for pseudouridylation is in bold.

FIG. 5 shows amplification of tRNA and mRNA targeting molecules using overlap PCR.

FIG. 6 shows pseudouridylated stop codons are decoded by a number of near- and non-cognate tRNAs. Three of the tRNAs, tRNA^{Lys}(UUU), tRNA^{Lys}(CUU), and tRNA^{Arg}(UCU), can form excellent base-pairing interactions with UAA, UAG, and UGA, respectively, where two (the second and third) are Watson-Crick base pair and one (the first) is a U-U pair. Both uridines of this pair are targeted by gRNAs for pseudouridylation (indicated).

FIG. 7 shows Box H/ACA gRNA-directed PTC (mRNA) pseudouridylation. (A) Base-pairing interactions are shown between the pseudouridylation pocket of PTC^{(K30X)}-gRNA (designer box H/ACA gRNA) and its substrate RNAs [PTC (UAA, UGA, and UAG)-containing cup1 mRNAs]. The target uridines (of PTCs) are indicated (underlined Ψs). (B) Total RNA was isolated from cells co-transformed with the cup1^{(K30UA)} gene and two of the following gRNA gene(s): two control gRNAs (lanes 1 and 2), a control gRNA and tRNA^{Lys}(UUU)-gRNA (TKu, lanes 3 and 4), a control gRNA and tRNA^{Lys}(CUU)-gRNA (TKc, lanes 5 and 6), a control gRNA and tRNA^{Arg}(UCU)-gRNA (TR, lanes 7 and 8), PTC^{(K30X)}-gRNA and a control gRNA (lanes 9 and 10), PTC^{(K30X)}-gRNA and tRNA^{Lys}(UUU)-gRNA (lanes 11 and 12), PTC^{(K30X)}-gRNA and tRNA^{Lys}(CUU)-gRNA (lanes 13 and 14), or PTC^{(K30X)}-gRNA and tRNA^{Arg}(UCU)-gRNA (lanes 15 and 16). After treatment with (lanes 2, 4, 6, 8, 10, 12, 14, 16) or without (lanes 1, 3, 5, 7, 9, 11, 13, 15) CMC and alkaline, primer-extension with a primer complementary to cup1 mRNA was carried out. The band in odd-numbered lanes was the fully extended product. The band in even-numbered lanes was the stop caused by the bulky CMC-modified Ψ. Lanes 17-20 are primer-extension sequencing ladders, which help locate the exact positions of the primer-extension stop bands (stopped one nucleotide before the CMC-Ψ sites). (C) The same experiments as in (B) were conducted, except that the cup1(K30UGA) gene (instead of the cup1^{(K30UAA)} gene) was used. (D) The same experiments as in (B) were performed, except that the cup1^{(K30UAG)} gene (instead of the cup1^{(K30UAA)} gene) was used. (E) The intensity of primer-extension stop bands was quantified using the data shown in (B), (C), and (D). Lanes 1-4 in (E) correspond to lanes 10, 12, 14 and 16 in (B); lanes 5-8 in (E) correspond to lanes 10, 12, 14 and 16 in (C); lanes 9-12 in (E) correspond to lanes 10, 12, 14 and 16 in (D).

FIG. 8 shows box H/ACA gRNA-directed tRNA pseudouridylation. (A) Base-pairing interactions are shown between the pseudouridylation pockets of tRNA-gRNAs (Sc-TKu, Sc-TKc and Sc-TR) and their substrate RNAs [anticodon sequences of pre-tRNA^{Lys}(UUU), tRNA^{Lys}(CUU), and tRNA^{Ar}(UCU), respectively]. The intron sequence of pre-tRNA^{Lys}(UUU) is shown in lowercase. Y: cytidine or uridine. (B) Total RNA was isolated from cells transformed with a control gRNA (lanes 1 and 2), tRNA^{Lys}(UUU)-gRNA (TKu, lanes 3 and 4), tRNA^{Lys}(CUU)-gRNA (TKc, lanes 5 and 6), tRNA^{Arg}(UCU)-gRNA (TR, lanes 7 and 8), treated with (lanes 2, 4, 6, and 8) or without (lanes 1, 3, 5, and 7) CMC, hydrolyzed with alkaline, and primer-extended with a primer complementary to tRNA^{Lys}(UUU). The CMC-Ψ-induced primer-extension stop is indicated by the arrow. Lanes 9-12 are primer-extension sequencing ladders, which help locate the exact position of the primer-extension stop band (stopped one nucleotide before the CMC-Ψ site). (C) The same experiments as in (B) were conducted, except that the primer-extension primer was complementary to tRNA^{Lys}(CUU) [instead of tRNA^{Lys}(UUU)]. (D) The same experiments as in (B) were conducted, except that the primer-extension primer was complementary to tRNA^{Arg}(UCU) [instead of tRNA^{Lys}(UUU)]. (E), (F), and (G) Quantification of the primer-extension data (the relative intensity of primer-extension stop bands) presented in (B), (C), and (D), respectively.

FIG. 9 shows quantitative analysis of targeted pseudouridylation. (A) Schematic diagram of the procedures. Site-specific RNase H cleavage of tRNA directed by a 2′-O-methyl RNA-DNA chimera (left) as well as site-specific cleavage of mRNA by DNAzyme 10-23 (right) are shown. The cleavage occurred 5′ of the target U (or Ψ if modified). ³²P was then introduced to the 5′ terminus of the 3′-half cleaved fragment. After nuclease P1 digestion, mononucleotides (including the ³²P-labeled target U or Ψ) were resolved on a TLC plate. (B) After transformation with a specific tRNA-gRNA, cells were lysed and the target tRNA isolated. The isolated tRNA was then subjected to quantification analysis, as described in (A). In lanes 3 and 4, tRNA^{Lys}(UUU) was isolated from cells transformed with a control (nonspecific) gRNA (lane 3) or with tRNA^{Lys}(UUU)-gRNA (TKu) (lane 4). In lanes 5 and 6, tRNA^{Arg}(UCU) was isolated from cells transformed with a control (nonspecific) gRNA (lane 5) or with tRNAArg(UCU)-gRNA (TR). In lanes 7-9, tRNA^{Lys}(CUU) was isolated from cells transformed with a control gRNA (lane 7) or with tRNA^{Lys}(CUU)-gRNA (TKc) (lane 8, one copy, and lane 9, three copies). Lane 1 is the markers for pU, pC, pA, and pG; lane 2 is the pΨ marker. The positions of pΨ are indicated by the arrows. (C) Quantification of the pseudouridylation data presented in (B). The lane numbers correspond to the lane numbers in (B). (D) After co-transformation with the cup1^{K30UAA} gene and a control gRNA (nonspecific) gene (lane 1) or with the cup1^{K30UAA} gene and the PTC(K30X)-gRNA gene (lane 2), cells were lysed and the cup1^{K30UAA} mRNA isolated. The isolated mRNA was then subjected to quantification analysis, as described in (A). Lane 3 shows the markers for pU, pC, pA, and pG; lane 4 is the pΨ marker. The arrow indicates the position of pΨ. (E) Quantification of the pseudouridylation data presented in (D). Lane 2 in (E) corresponds to lane 2 in (D).

FIG. 10 shows cup1-nonsense suppression assay verifying the Ψ-Ψ codon-anticodon pair. (A) The strategy for Ψ-Ψ-mediated nonsense suppression is depicted. The construct with a cup1gene containing a PTC (TAA, TAG or TGA) at codon 30 (K30X) is shown. Also shown are the promoter, a 3×FLAG tag, and the terminator (or the PolyA signal). Both the cup1mRNA and a tRNA (as a matching pair or a mismatching pair) were targeted (or mistargeted) for pseudouridylation by gRNAs. The RNA genes were co-transformed into cup1Δ cells. Cells were plated on media containing different concentrations of copper. Cell growth/survival was assessed. (B) cup1Δ cells were transformed with the wild-type CUP1 (with no PTC) gene, along with two control (nonspecific) gRNAs (Panel A) or a control gRNA and PTC^{(K30X)}-gRNA (Panel B). (C) cup1Δ cells were co-transformed with the cup1^{K30UAA} gene and the following gRNA genes: two control gRNAs (nonspecific) (Panel A), a control gRNA and tRNA^{Lys}(UUU)-grNA (TKu) (Panel B), a control gRNA and tRNA^{Lys}(CUU)-gRNA (TKc) (Panel C), a control gRNA and tRNA^{Arg}(UCU)-gRNA (TR) (Panel D), a control gRNA and PTC(K30X)-gRNA (Panel E), tRNA^{Lys}(UUU)-grNA (TKu) and a PTC^{(K30UAA)}-gRNA (Panel F), tRNA^{Lys}(CUU)-gRNA (TKc) and a PTC^{(K30X)}-gRNA (Panel G), or tRNA^{Arg}(UCU)-gRNA (TR) and PTC^{(K30X)}-gRNA (Panel H). The copper concentrations are indicated on the left. (D) The same experiments as in (C) were carried out, except that the cup1^{K30UGA} gene (rather than the cup1^{K30UAA} gene) was used. (E) The same experiments as in (C) were carried out, except that the cup1^{K30UAG} gene (rather than the cup1^{K30UAA} gene) was used.

FIG. 11 shows nonsense suppression assay verifying the Ψ-Ψ codon-anticodon pair in the mCherry gene context. (A) Strategy is the same as in FIG. 10A, except that a mCherry gene containing a PTC (TAA, TAG or TGA) at codon 30 (K30X) was transformed, and fluorescence imaging (rather than cell growth in copper containing media) was used. (B) Yeast cells were co-transformed with the wild-type mCherry gene (with no PTC) and two control gRNA genes (Panel 1) or a control tRNA-gRNA gene and a PTC^{(K30UAA)}-specific gRNA gene (Panel 2). (C) Yeast cells were co-transformed with the PTC-containing mCherry gene, either PTC^{(K30UAA)} (Panels 1-4), PTC^{(K30UGA)} (Panels 5-8), or PTC^{(K30UAG)} (panels 9-14), and two additional gRNA genes: two control gRNA genes (Panels 1, 5 and 9), a control PTC-gRNA gene (nonspecific) and a tRNA^{Lys}(UUU)-gRNa gene (TKu) (Panel 2), a control PTC-gRNA gene and a tRNA^{Arg}(UCU)-gRNA (TR) (Panel 6), a control PTC-gRNA gene and a tRNA^{Lys}(CUU)-gRNA (TKc) (Panel 10, one copy; Panel 13, three copies), a control tRNA-gRNA gene (nonspecific) and a PTC-specific gRNA gene (Panels 3, 7, and 12), a PTC^{(K30UAA)}-specific gRNA gene and a tRNA^{Lys}(UUU)-gRNa gene (TKu) (Panel 4), a PTC^{(K30UGA)}-specific gRNA gene and a tRNA^{Arg}(UCU)-gRNA gene (TR) (Panel 8), or a PTC^{(K30UAG)}-specific gRNA gene and a tRNA^{Lys}(CUU)-gRNA gene (TKc) (Panel 13, one copy; and Panel 14, 3 copies). Exposure times are shown at the bottom. (D), (E), and (F) Quantification of the fluorescence data derived from (B) and (C), Panels 1-4, Panels 5-8, and Panels 9-14, respectively. Lane 1, the wild type data shown in (B); lanes 2-5 in (D) correspond to Panels 1-4 in (C), respectively; lanes 2-5 in (E) correspond to Panels 5-8 in (C), respectively; and lanes 2-7 in (F) correspond to Panels 9-14 in (C), respectively. (G), (H), and (I) Western blot analysis of samples derived from (B) and (C). Lane 1, total protein (cell lysate) was derived from cells transformed with wild-type mCherry as in (B). Total protein in lanes 2-5 in (G) was derived from samples corresponding to Panels 1-4 in (C), respectively; total protein in lanes 2-5 in (G) was derived from samples corresponding to Panels 1-4 in (C), respectively; total protein in lanes 2-5 in (H) was derived from samples corresponding to Panels 5-8 in (C), respectively; total protein in lanes 2-7 in (I) was derived from samples corresponding to Panels 9-14 in (C), respectively.

FIG. 12 shows melting curve analysis for the Ψ-Ψ pair. (A) A well-studied RNA duplex was used. The sequences of the two strands are shown, where x-y stands for either U-U, Ψ-U, Ψ-Ψ, U-A, or Ψ-A base-pair. (B) and (C) Melting curve was measured in 1 M Na+ and 20 mM phosphate buffer, pH=7, where the RNA duplex concentration was 10 μM. The melting temperatures of the duplex with different x-y pairs were determined.

FIG. 13 shows nonsense suppression assay verifying the Ψ-Ψ codon-anticodon pair in the mCherry gene context in human cells. (A) Base-pairing interactions are shown between the pseudouridylation pocket of Hs-PTC^{(K14X)}-gRNA and its substrate RNAs [PTC (UAA/UGA)-containing mCherry mRNAs] and between HstRNA-gRNAs (Hs-TKu and Hs-TKc) and their substrate RNAs [tRNA^{Lys}(UUU) and tRNA^{Lys}(CUU), respectively]. The target uridines (of PTCs or anticodons) are indicated (red Ψs). R: adenosine or guanosine; Y: cytidine or uridine. (B) HEK-293 cells were co-transfected with the wild-type mCherry gene (with no PTC) and two control gRNA genes (Panel 1) or a control tRNA-gRNA gene and a PTC^{(K14UAA)}-specific gRNA gene (Panel 2). (C) HEK-293 cells were co-transfected with the PTC-containing mCherry gene, either PTC^{(K14UAA)} (Panels 1-6) or PTC^{(K14UAG)} (Panels 7-12), and two additional gRNA genes: two control gRNA genes (Panels 1 and 7), a control PTC-gRNA gene (nonspecific) and a tRNA^{Lys}(UUU)-gRNA gene (TKu) (Panels 2 and 8), a control PTC-gRNA gene and a tRNA^{Lys}(CUU)-gRNA (TKc) (Panels 3 and 9), a control tRNA-gRNA gene (nonspecific) and a PTC-specific gRNA gene (Panels 4 and 10), a PTC-specific gRNA gene (Hs-PTC^{(K14X)}-gRNA) and a tRNA^{Lys}(UUU)-gRNA gene (TKu) (Panels 5 and 11), or a PTC-specific gRNA gene (Hs-PTC^{(K14X)}-gRNA) and a tRNA^{Lys}(CUU)-gRNA gene (TKc) (Panels 6 and 12). Exposure times are shown at the bottom. (D) and (E) Western blot analysis of samples derived from (B) and (C). Lane 1, total protein (cell lysate) was derived from cells transformed with wild-type mCherry as in (B). Total protein in lanes 2-7 in (D) was derived from samples corresponding to Panels 1-6 in (C), respectively; total protein in lanes 2-5 in (E) was derived from samples of Panels 7-12 in (C), respectively.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a”, an and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself.

For example, if the value “10” is disclosed, then “about 10” is also disclosed. It is also understood that when a value is disclosed that “less than or equal to the value”, “greater than or equal to the value” and possible ranges between values are also disclosed, as appropriately understood by the skilled artisan.

This application is directed to methods of treatment of disease by nonsense suppression by targeting the anticodons of three tRNAs, tRNA^{Lys} (CUU) (for UAG), tRNA^{Lys}(UUU) (for UAA) and tRNA^{Arg}(UCU) (for UGA). The method comprises nonsense suppression during translation of the mRNA when both the mRNA and the tRNA are pseudouridylated. The method comprises nonsense codon ΨAG pairs with anticodon CUΨ, ΨAA with UUΨ, and ΨGA with UCΨ.

It has been shown that targeting the premature translation termination codon (PTC) (caused by “nonsense mutations”) within the mRNA coding region leads to nonsense suppression. However, the suppression efficiency is relatively low. In the present application, it is shown that there can be unexpectedly significant improvement in nonsense suppression by targeting the anticodons of three tRNAs, tRNA^{Lys} (CUU) (for UAG), tRNA^{LYs} (UUU) (for UAA) and tRNA^{Arg}(UCU) (for UGA). This application further shows that nonsense suppression is the most efficient if both the PTC (UAG, UAA, or UGA) and the tRNA anticodon are pseudouridylated, i.e., nonsense codon FAG pairs with anticodon CUΨ, ΨAA with UUΨ, and ΨGA with UCΨ. These results indicate that the Ψ-Ψ pair is much stronger than the U-U or U-Ψ pair. In addition, this application has verified, through mass spectrometry-based sequencing, that Arginine (brought by tRNA^{Arg}(UCU)) is incorporated at the pseudouridylated nonsense codon ΨGA. Experiments verify the Lysine incorporation at the FAG and ΨAA codons.

The application is further directed to a method of treating premature translation termination codon (PTC)-related diseases by targeting both the PTC on the mRNA and tRNAs that may react with the PTC. In some embodiments, the method comprises introducing pseudouridylation at the uridine residue of the PTC and at the corresponding uridine residue in the anticodon of certain tRNAs, to promote suppression of PTCs (nonsense suppression) during translation of the mRNA. In certain embodiments, the application describes artificially modified tRNAs that comprises a modified uridine residue at the anticodon region. In certain embodiments, the application describes methods for treating a PTC-related disease by (1) pseudouridylation of the uridine residue of the relevant PTC and (2) modifying the uridine residue of certain tRNAs that would lead to nonsense suppression. In certain embodiments, the application describes methods for reading through a PTC in an mRNA. In certain embodiments, the application describes a mRNA/tRNA complex, comprising: a modified mRNA comprising a premature translation termination codon (PTC), wherein the uridine in the PTC has been converted to a pseudouridine; and a modified tRNA, wherein the tRNA comprises a modified uridine at the N36 position of the anticodon sequence of the tRNA, wherein the pseudouridine in the modified mRNA pairs with the modified uridine in the anticodon sequence of the modified tRNA. In certain embodiments, the application describes a kit for treating a PTC-related disease.

### I. Definitions

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “nucleotide sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “promoter” as used herein is defined as a DNA sequence recognized by the synthetic machinery of the cell, or introduced synthetic machinery, required to initiate the specific transcription of a polynucleotide sequence.

As used herein, the term “regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

An “expression cassette” is a distinct component of vector DNA consisting of a gene and regulatory sequence to be expressed by a transfected cell. In each successful transformation, the expression cassette directs the cell's machinery to make RNA and protein(s). Some expression cassettes are designed for modular cloning of protein-encoding sequences so that the same cassette can easily be altered to make different proteins.

As used herein, the term “effective amount” is an amount necessary or sufficient to achieve the desired biological effect or the selected result, and such amount can be determined by one of ordinary skill in the art as a matter of routine experimentation.

The terms ‘adenine’, ‘guanine’, ‘cytosine’, ‘thymine’, ‘uracil’ and ‘hypoxanthine’ (the nucleobase in inosine) as used herein refer to the nucleobases as such.

The terms ‘adenosine’, ‘guanosine’, ‘cytidine’, ‘thymidine’, ‘uridine’, ‘pseudouridine’ and ‘inosine’, refer to the nucleobases linked to the (deoxy)ribosyl sugar.

The term ‘nucleoside’ refers to the nucleobase linked to the (deoxy)ribosyl sugar.

The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, and the like.

Sometimes the terms adenine and adenosine, guanine and guanosine, cytosine and cytidine, uracil and uridine, thymine and thymidine, inosine and hypoxanthine, are used interchangeably to refer to the corresponding nucleobase, nucleoside or nucleotide. Pseudouridine is often referred to as Ψ, or as 5-ribosyluridine.

Sometimes the terms nucleobase, nucleoside and nucleotide are used interchangeably, unless the context clearly requires differently. The terms ‘ribonucleoside’ and ‘deoxyribonucleoside’, or ‘ribose’ and ‘deoxyribose’ are as used in the art.

Whenever reference is made to an ‘oligonucleotide’, both oligoribonucleotides and deoxyoligoribonucleotides are meant unless the context dictates otherwise. Whenever reference is made to an ‘oligoribonucleotide’ it may comprise the bases A, G, C, U or I.

Whenever reference is made to a ‘deoxyoligoribonucleotide’ it may comprise the bases A, G, C, T or I. In certain embodiments, the editing oligonucleotide of the present application is an oligoribonucleotide that may comprise chemical modifications, and may include deoxynucleotides (DNA) at certain specified positions.

Whenever reference is made to nucleosides in the oligonucleotide, such as cytidine, 5-methylcytosine, 5-hydroxymethylcytosine, pyrrolocytidine, and β-D-glucosyl-5-hydroxymethylcytidine are included; when reference is made to adenosine, 2-aminopurine, 2,6-diaminopurine, 3-deazaadenosine, 7-deazaadenosine, 8-azidoadenosine, 8-methyladenosine, 7-aminomethyl-7-deazaguanosine, 7-deazaguanosine, N(6)-methyladenosine and 7-methyladenosine are included; when reference is made to uridine, 5-methoxyuridine, 5-methyluridine, dihydrouridine, pseudouridine, and thienouridine, dihydrouridine, 4-thiouridine and 5-hydroxymethyluridine are included; when reference is made to guanosine, 7-methylguanosine, 8-aza-7-deazaguanosine, thienoguanosine and 1-methylguanosine are included. One of ordinary skill in the art will understand that nucleobases (e.g., cytosine, adenine, uracil, and guanine) may also be used in certain embodiments.

Whenever reference is made to nucleosides or nucleotides, ribofuranose derivatives, such as 2′-deoxy, 2′-hydroxy, 2′-fluoro, and 2′-O -substituted variants, such as 2′-O-methyl (2′-OMe), are included, as well as other modifications, including 2′-4′ bridged variants.

Whenever reference is made to oligonucleotides, linkages between two mono-nucleotides may be phosphodiester linkages as well as modifications thereof, including, phosphodiester, phosphotriester, phosphoro(di)thioate, methylphosphonate, phosphor-amidate linkers, and the like.

The term ‘comprising’ encompasses ‘including’ as well as ‘consisting’, e.g. a composition ‘comprising X’ may consist exclusively of X or may include something additional, e.g. X+Y.

The term ‘about’ in relation to a numerical value x is optional and means, e.g. x±10%.

The word ‘substantially’ does not exclude ‘completely’, e.g. a composition which is ‘substantially free from Y’ may be completely free from Y. Where relevant, the word ‘substantially’ may be omitted from the definition of the invention.

The term “complementary” as used herein refers to the fact that the nucleic acid molecule according to the invention hybridizes under physiological conditions to the target RNA sequence and/or to its own internal sequences, especially within the hairpin structure. The term does not mean that each and every nucleotide in the nucleic acid molecule has a perfect pairing with its opposite nucleotide in the target sequence or within the hairpin structure. In other words, while a nucleic acid molecule according to the invention may be complementary to a target sequence, there may be mismatches, wobbles and/or bulges between the nucleic acid molecule of the present application and the target sequence, while under physiological conditions that nucleic acid molecule still hybridizes to the target sequence such that the cellular enzymes can convert the target uridine to a Ψ. The term “substantially complementary” therefore also means that in spite of the presence of the mismatches, wobbles, and/or bulges, the nucleic acid molecule according to the present application has enough matching nucleotides with the target sequence that under physiological conditions the nucleic acid molecule hybridizes to the target RNA. As shown herein, a nucleic acid molecule may be complementary, but may also comprise one or more mismatches, wobbles and/or bulges with the target sequence, as long as under physiological conditions the nucleic acid molecule of the present application is able to hybridize to its target.

The term ‘downstream’ in relation to a nucleic acid sequence means further along the sequence in the 3′ direction; the term ‘upstream’ means the converse. Thus in any sequence encoding a polypeptide, the start codon is upstream of the stop codon in the sense strand, but is downstream of the stop codon in the antisense strand.

References to ‘hybridization’ typically refer to specific hybridization, and exclude non-specific hybridization. Specific hybridization can occur under experimental conditions chosen, using techniques well known in the art, to ensure that the majority of stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. Mismatching nucleotides are G-A, C-A, U-C, A-A, G-G, C-C, U-U pairs. In some embodiments nucleic acid molecules according to the present application comprise fewer than four mismatches, for example 0, 1 or 2 mismatches.

A wobble pair, or wobble base pair, is a hydrogen-bonded pairing between two nucleotides generally occurring between two RNA molecules. These base pairs are geometrically distinct from the canonical Watson-Crick-type base-pairing. The main wobble base pairs are hypoxanthine-uracil (I-U, where I represents inosine, the nucleoside formed from hypoxanthine), guanine-uracil (G-U), hypoxanthine-adenine (I-A), and hypoxanthine-cytosine (I-C). Wobble base pairs are of comparable thermodynamic stability to Watson-Crick base pairs, Wobble base pairs occur frequently in RNA secondary structure and are important for proper translation of the genetic code.

A Ψ-Ψ pair is a hydrogen-bonded pairing between two pseudouridines. The inventors have surprisingly discovered that the Ψ-Ψ pair is much stronger than a U-U or U-Ψ pair and can be used to facilitate nonsense codon recognition and alter the specificity of a sense codon could also be altered.

### II. Modified tRNA

One aspect of the present application relates to artificially modified tRNA molecules that are capable of suppressing a stop codon (i.e., extending a peptide chain through a stop codon). In some embodiments, the artificially modified tRNA comprises a modified anticodon sequence N34N35N36 that comprises a modified uridine at position 36 (N36) of the anticodon sequence.

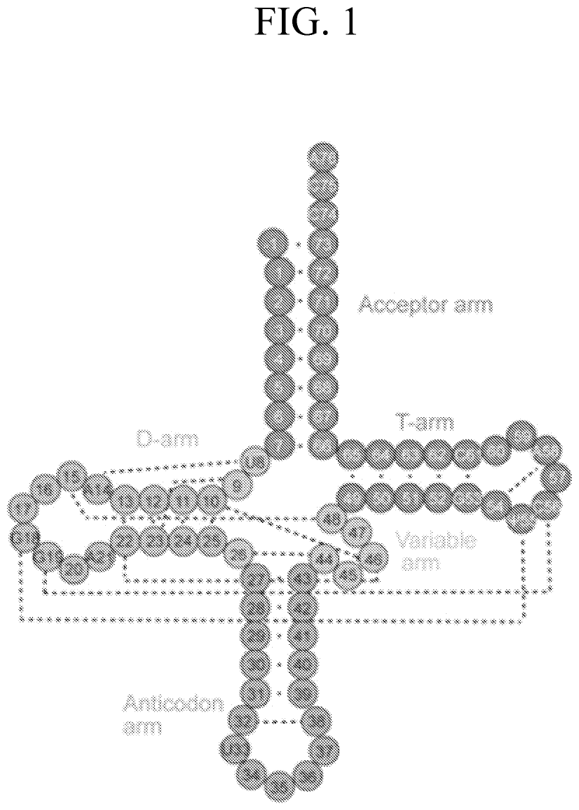

FIG. 1 shows the two-dimensional structure of a tRNA, which consists from 5′ end to 3′ end, an acceptor stem, a D-arm, an anticodon arm, a variable arm, a T-arm, the acceptor stem with a CCA tail.

The acceptor arm is a 7- to 9-base pair (bp) stem made by the base pairing of the 5′-terminal nucleotide with the 3′-terminal nucleotide (which contains the CCA 3′-terminal group used to attach the amino acid). In general, such 3′-terminal tRNA-like structures are referred to as ‘genomic tags’. The acceptor stem may contain non-Watson-Crick base pairs.

The D-arm is a 4- to 6-bp stem ending in a loop (D-loop) that often contains dihydrouridine (D).

The anticodon arm is a 5-bp stem whose loop (anticodon loop) contains the anticodon, which is located at the N34, N35 and N36 positions of the tRNA molecule. The tRNA 5′-to-3′ primary structure contains the anticodon but in reverse order, since 3′-to-5′ directionality is required to read the mRNA from 5′-to-3′. In other words, a tRNA with the anticodon sequence at N34N35N36 position would pair with a mRNA with a codon at N1N2N3 position in the following manner:

tRNA anticodon
3′- N36 N35 N34 -5′
mRNA codon
5′- N1 N2 N3 - 3′

The variable arm sits next to the anticodon arm and varies in size from 3 to 21 bases.

The T-arm is a 5-bp stem ending in a loop (T-loop or TΨC loop) that often contains one or more unusual base, such as 5-methyluridine (m⁵U or T) and pseudouridine (Ψ).

The CCA tail is a cytidine-cytidine-adenosine sequence at the 3′ end of the tRNA molecule. The amino acid loaded onto the tRNA by aminoacyl tRNA synthetases, to form aminoacyl-tRNA, is covalently bonded to the 3′-hydroxyl group on the CCA tail. This sequence is important for the recognition of tRNA by enzymes and critical in translation. In prokaryotes, the CCA sequence is transcribed in some tRNA sequences. In most prokaryotic tRNAs and eukaryotic tRNAs, the CCA sequence is added during processing and therefore does not appear in the tRNA gene.

In certain embodiments, the modified uridine at position 36 (N36) (if consider sense codons, positions 34 and 35 as well) of the artificially modified tRNA of the present application is pseudouridine (Ψ).

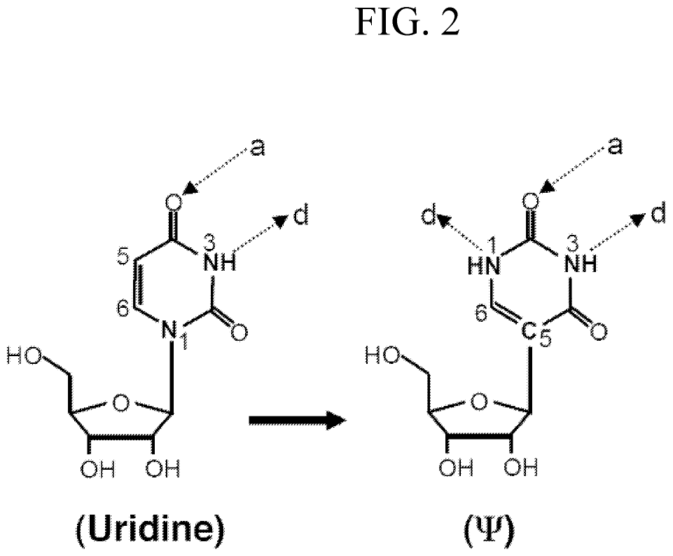

FIG. 2 shows the structures of uridine and T. In some embodiments, the artificially modified tRNAs of the present application comprise an artificially modified tRNA^{Lys}(UUU) that comprises a pseudouridine (Ψ) at position N36. A representative gene sequence for Saccharomyces cerevisiae wild-type tRNA^{Lys} (UUU) (gene symbol: YNCD0003W) is

(SEQ ID NO: 1)
UCCUUGUUAGCUCAGUUGGUAGAGCGUUCGGCUUUUAACCGAAAUGUCA
GGGGUUCGAGCCCCCUAUGAGGAG.

In some embodiments, the artificially modified tRNAs of the present application comprise an artificially modified tRNA^{Lys}(CUU) that comprises a pseudouridine (Ψ) at position 36. A representative gene sequence for Saccharomyces cerevisiae wild-type tRNA^{Lys} (CUU) (gene symbol: YNCC0009C) is

(SEQ ID NO: 2)
GCCUUGUUGGCGCAAUCGGUAGCGCGUAUGACUCUUAAUCAUAAGGUUA
GGGGUUCGAGCCCCCUACAGGGCU.

In some embodiments, the artificially modified tRNAs of the present application comprise an artificially modified tRNA^{Arg} (UCU) that comprises a pseudouridine (Ψ) at position 36. A representative gene sequence for Saccharomyces cerevisiae wild-type tRNA^{Ag}(UCU) (gene symbol: YNCB0015W) is GCUCGCGUGGCGUAAUGGCAACGCGUCUGACUUCUAAUCAGAAGAUUAUGGGUU CGACCCCCAUCGUGAGUG (SEQ ID NO:3). This tRNA naturally has a shortened D loop; this does not affect numbering, so that the anticodon sequence will always be annotated with N34N35N36.

In certain embodiments, the artificially modified tRNA is an artificially modified human tRNA with low tissue specific expression, wherein the uridine (U) at position 36 (N36) is replaced with pseudouridine (Ψ).

Examples of human tRNA with low tissue specific expression include, but are not limited to,

tRNA^{Lys}(UUU): gene symbol: TRK-TTT3-1
(SEQ ID NO: 4)
GCCCGGAUAGCUCAGUCGGUAGAGCAUCAGACUUUUAAUCUGAGGGUCC
AGGGUUCAAGUCCCUGUUCGGGCG
tRNA^{Lys}(CUU): gene symbol: TRK-CTT2-1
(SEQ ID NO: 5)
GCCCGGCUAGCUCAGUCGGUAGAGCAUGAGACUCUUAAUCUCAGGGUCG
UGGGUUCGAGCCCCACGUUGGGCG
tRNA^{Arg}(UCU): gene symbol: TRR-TCT3-1
(SEQ ID NO: 6)
GGCUCUGUGGCGCAAUGGAUAGCGCAUUGGACUUCUAAUUCAAAGGUUG
UGGGUUCGAGUCCCACCAGAGUCG

In certain embodiments, the modified tRNA is an S. cerevisiae tRNA^{Lys} having a modified anticodon of UUΨ. In certain embodiments, the modified S. cerevisiae tRNA^{Lys} has a sequence of

(SEQ ID NO: 13)
UCCUUGUUAGCUCAGUUGGUAGAGCGUUCGGCUUUΨAACCGAAAUGUCA
GGGGUUCGAGCCCCCUAUGAGGAG.

In certain embodiments, the modified tRNA is an S. cerevisiae tRNA^{Lys} having a modified anticodon of CUΨ. In certain embodiments, the modified S. cerevisiae tRNA^{Lys} has a sequence of

(SEQ ID NO: 14)
GCCUUGUUGGCGCAAUCGGUAGCGCGUAUGACUCUΨAAUCAUAAGGUUA
GGGGUUCGAGCCCCCUACAGGGCU.

In certain embodiments, the modified tRNA is an S. cerevisiae tRNA^{Arg} having a modified anticodon of UCΨ. In certain embodiments, the modified S. cerevisiae tRNA^{A}g has a sequence of

(SEQ ID NO: 15)
GCUCGCGUGGCGUAAUGGCAACGCGUCUGACUUCΨAAUCAGAAGAUUAU
GGGUUCGACCCCCAUCGUGAGUG.

In certain embodiments, the modified tRNA is a human tRNA^{Lys} having a modified anticodon of UUP. In certain embodiments, the modified human tRNA^{Lys} has a sequence of

(SEQ ID NO: 16)
GCCCGGAUAGCUCAGUCGGUAGAGCAUCAGACUUUΨAAUCUGAGGGUCC
AGGGUUCAAGUCCCUGUUCGGGCG

In certain embodiments, the modified tRNA is a human tRNA^{Lys} having a modified anticodon of CUT. In certain embodiments, the modified human tRNA^{Lys} has a sequence of

(SEQ ID NO: 17)
GCCCGGCUAGCUCAGUCGGUAGAGCAUGAGACUCUΨAAUCUCAGGGUCG
UGGGUUCGAGCCCCACGUUGGGCG

In certain embodiments, the modified tRNA is a human tRNA^{Arg} having a modified anticodon of UCΨ. In certain embodiments, the modified human tRNA^{Arg} has a sequence of

(SEQ ID NO: 18)
GGCUCUGUGGCGCAAUGGAUAGCGCAUUGGACUUCΨAUUCAAAGGUUGU
GGGUUCGAGUCCCACCAGAGUCG

In some embodiments, the artificially modified tRNA molecules of the present application further comprise one or more modifications in one or more nucleotides of the modified tRNA. Examples of modifications include, but not limited to, pseudouridylation, 2′-O-methyl modified sugar moiety, a 2′-O-methoxyethyl modified sugar moiety, a phosphorothioate internucleoside linkage, or a phosphodiester internucleoside linkage, or a combination thereof.

In certain embodiments, the one or more further modifications are at positions other than position 36 (N36).

In some embodiments, the artificially modified tRNAs of the present application are synthesized chemically or generated in vitro.

In some embodiments, the artificially modified tRNAs are created in vivo. The conversion of uridine to Ψ (pseudouridylation) requires two distinct chemical reactions: the breaking of the C1′-N1 glycosidic bond and the making of a new carbon (C1′-C5) bond that relinks the base to the sugar. Pseudouridylation is a true isomerization reaction, which creates an extra hydrogen bond donor and thereby influences a wide variety of functional aspects depending on the type of RNA that carries the Ψ and the position within the RNA sequence, such as protein synthesis, increased stop-codon read-through and frame shifting. Using pseudouridylation, an RNA molecule in a cell can be targeted by a nucleic acid molecule, such as an oligonucleotide, to recruit a pseudouridine synthase to convert a specific uridine present in the RNA sequence into pseudouridine; more specifically, oligonucleotides and intron-embedded snoRNAs can promote pseudouridylation of a uridine in a target RNA (see WO2019/191232).

### II. Methods of Treating Diseases Relating to Premature Translation Termination Codon

Another aspect of the present application relates a method of treating a premature translation termination codon N1N2N3 (PTC)-related disease in a subject. The method comprises the steps of: a) modifying a PTC in an mRNA in a cell of the subject by converting a uridine (U) residue in the PTC into a pseudouridine (Ψ) to create a modified mRNA, wherein the PTC results in the PTC-related disease, wherein the uridine is at position 1 (N1) in the PTC; b) providing a modified tRNA in the cell, wherein the modified tRNA comprises a modified uridine at position N36 of an anticodon sequence (N34, N35, N36); wherein the modified uridine in the modified tRNA is capable of pairing with the pseudouridine (Ψ) in the modified mRNA to attach an amino acid to the translation of the modified mRNA and produce a full length translation product from the modified mRNA.

Examples of PTC-related diseases include, but are not limited to, cystic fibrosis, Hurler syndrome, alpha-1-antitrypsin (A1AT) deficiency, Parkinson's disease, Alzheimer's disease, albinism, amyotrophic lateral sclerosis, asthma, β-thalassemia, Cadasil syndrome, Charcot-Marie-Tooth disease, chronic obstructive pulmonary Disease (COPD), distal spinal muscular atrophy (DSMA), Duchenne/Becker muscular dystrophy, dystrophic epidermolysis bullosa, epidermylosis bullosa, Fabry disease, factor V Leiden associated disorders, familial adenomatous, polyposis, galactosemia, Gaucher's disease, glucose-6-phosphate dehydrogenase, haemophilia, hereditary hematochromatosis, Hunter syndrome, Huntington's disease, inflammatory bowel disease (IBD), inherited polyagglutination syndrome, Leber congenital amaurosis, Lesch-Nyhan syndrome, Lynch syndrome, Marfan syndrome, mucopolysaccharidosis, muscular dystrophy, myotonic dystrophy types I and II, neurofibromatosis, Niemann-Pick disease type A, B and C, NY-eso1 related cancer, Peutz-Jeghers Syndrome, phenylketonuria, Pompe's disease, primary ciliary disease, prothrombin mutation related disorders, such as the prothrombin G20210A mutation, pulmonary hypertension, (autosomal dominant), retinitis pigmentosa, Sandhoff disease, severe combined immune deficiency syndrome (SCID), sickle cell anemia, spinal muscular atrophy, Stargardt's disease, Tay-Sachs disease, Usher syndrome, X-linked immunodeficiency, Sturge-Weber syndrome, cancer and neurofibromatosis.

### Modifying PTC

In some embodiments, the step of modifying a PTC comprises the substep of introducing a mRNA targeting nucleic acid molecule for pseudouridylation into the cell, wherein the mRNA targeting nucleic acid molecule comprises a guide region capable of forming a partially double stranded nucleic acid complex with the mRNA, wherein the partially double stranded nucleic acid complex is capable of engaging a mammalian pseudouridylation enzyme, wherein the guide region assists in positioning the uridine in the PTC in the partially double stranded nucleic acid complex for it to be converted to a pseudouridine by the mammalian pseudouridylation enzyme.

In some embodiments, the mRNA targeting nucleic acid molecule for pseudouridylation that is capable of introducing pseudouridylation of the uridine at the N1 position of the PTC. Designing and expression of such mRNA targeting nucleic acid molecules are described in more details in PCT/US2019/024282, incorporated herein by reference.

Examples of the mRNA targeting nucleic acid molecule for pseudouridylation include, but are not limited to, GGCACAACCUUCUGACCUGCUUUCUCUUAUGUGAGUAGUGUUAAGAACUAUGUG CUAUACAAAUAAUUGCAGCACCUUCUGCAGUAUAACUAUAAAUAGUAAUGCUGC AAGAACUCUGCAGACAAAA (SEQ ID NO:19). This particular sequence targets the pseudouridylation on CFTR gene with G542X nonsense mutation at N1 of the PTC.

In some embodiments, the mRNA targeting nucleic acid molecule is a synthetic molecule that is introduced into the cell using methods well known in the art, such as liposome mediated transfection or electroporation. In other embodiments, the mRNA targeting nucleic acid molecule is expressed inside the cell using an expression vector, such as a plasmid vector or viral vector. Examples of viral vectors include, but are not limited to, adenovirus vectors, adeno-associated virus (AAV) vectors, retrovirus vectors and lentivirus vectors.

Modifying tRNA

In some embodiments, the step of providing a modified tRNA in the cell comprises the substep of introducing the artificially modified tRNA of the present application into the cell. In some embodiments, the artificially modified tRNA of the present application is a synthetic RNA. In some embodiments, the synthetic RNA is introduced into the cell using technologies know in the art, such as nanoparticle/liposome mediated transfection and electroporation.

In some embodiments, the step of providing a modified tRNA in the cell comprises the substep of introducing a tRNA targeting nucleic acid molecule for pseudouridylation into the cell, wherein the tRNA targeting nucleic acid molecule comprises a guide region capable of forming a partially double stranded nucleic acid complex with a tRNA in the cell, wherein the partially double stranded nucleic acid complex is capable of engaging a mammalian pseudouridylation enzyme, wherein the guide region assists in positioning the uridine in the tRNA in the partially double stranded nucleic acid complex for it to be converted to a pseudouridine by the mammalian pseudouridylation enzyme inside the cell.

In some embodiments, the tRNA targeting nucleic acid molecule for pseudouridylation is a pseudouridylating editing oligonucleotide (psEON) that is capable of introducing pseudouridylation of the uridine at the N36 position of the tRNA.

Examples of tRNA targeting nucleic acid molecule for pseudouridylation include, but are not limited to,

Saccharomyces cerevisiae:

Molecule for tRNA^{Lys}(UUU):
(SEQ ID NO: 7)
GGGACUGAAUGCGCUCCGGCGAGGCAGCCCACAUCAAGUGGAACUACAC
AGACUUCCUUGUCGCAAAGCCGCGGUCCCAAGAGCAAUCCUUCCCGAUG
CGCUGCUCAUCUAUUAAAAUCAAUAGUAGAAUGUAGUAAAGCCGACGGG
AGACAUUC
Molecule for tRNA^{Lys}(CUU):
(SEQ ID NO: 8)
GGGACUGCUUAUGAUCCGGCGAGGCAGCCCACAUCAAGUGGAACUACAC
AGACUUCCUUGUCGCAGAGUCACGGUCCCAAGAGCAAUCCUUCCCGUUA
UGAUGCUCAUCUAUUAAAAUCAAUAGUAGAAUGUAGUAGAGUCAUCGGG
AGACAUUC
Molecule for tRNA^{Arg}(UCU):
(SEQ ID NO: 9)
GGGACCUCUUCUGAUACGGCGAGCCUGCCCACAUCAAGUGGAACUACAC
AGACAUGCUUGUCGCGAAGUCACGGUCCCAAGAGCAAUCCUUCCCGUUC
UGAUGCUCAUCUAUUAAAAUCAAUAGUAGAAUGUAGUGAAGUCAGCGGG
AGACAUUC.

Homo sapiens:

Molecule for tRNA^{Lys}(UUU):
(SEQ ID NO: 10)
CCACACACCUCAGAUCCAGUCCAGGGCAGCUUCCCUGUUCUGCAAAGUC
UUGUGUGGACAUUAAAAUUGCGACCUCAGAUCCAGUAUAACUAUAAAUA
GUAAUGCUGCAAAGUCUUCGCAGACAAAA.
Molecule for tRNA^{Lys}(CUU):
(SEQ ID NO: 11)
CCACACACCUGAGAUCCAGUCCAGGGCAGCUUCCCUGUUCUGCAGAGUC
UUGUGUGGACAUUAAAAUUGCGACCUGAGAUCCAGUAUAACUAUAAAUA
GUAAUGCUGCAGAGUCUUCGCAGACAAAA.
Molecule for tRNA^{Arg}(UCU):
(SEQ ID NO: 12)
CCACACACUUUGGAUACAGUCCAGGGCAGCUUCCCUGUUCUGCGAAGUC
CUGUGUGGACAUUAAAAUUGCCUCUUUGGAUACAGUAUAACUAUAAAUA
GUAAUGCUGCGAAGUCCAGGCAGACAAAA.

In some embodiments, the tRNA targeting nucleic acid molecule is a synthetic molecule that is introduced into the cell using methods well known in the art, such as liposome mediated transfection or electroporation. In other embodiments, the tRNA targeting nucleic acid molecule is expressed inside the cell using an expression vector, such as a plasmid vector or viral vector. In some embodiments, sequences encoding the tRNA targeting molecules and the mRNA targeting molecules are cloned into the same expression vector and are co-expressed inside the cell from the expression vector.

In some embodiments, the modified tRNA molecules of the present application further comprise one or more modifications in one or more nucleotides of the modified tRNA. Examples of modifications include, but not limited to, pseudouridylation, 2′-O-methyl modified sugar moiety, a 2′-O-methoxyethyl modified sugar moiety, a phosphorothioate internucleoside linkage, or a phosphodiester internucleoside linkage, or a combination thereof.

### III. Method for Reading Through a PTC in an mRNA

Another aspect of the present application relates to a method of reading through a premature translation termination codon N1N2N3 (PTC) in an mRNA comprising: a) modifying the PTC in the mRNA in a cell by converting a uridine (U) residue in the PTC into a pseudouridine (Ψ) to create a modified mRNA, wherein the uridine is at position 1 (N1) in the PTC; and b) providing a modified tRNA in the cell, wherein the modified tRNA comprises a modified uridine at the last position (position 36 or N36) of an anticodon sequence (N34N35N36) of the modified tRNA, wherein the modified uridine in the modified tRNA is capable of paring with the pseudouridine (Ψ) in the modified mRNA to incorporate an amino acid at the modified PTC and produce a full length translation product from the modified mRNA.

Methods for modifying the PTC in the mRNA by converting a uridine (U) residue in the PTC into a pseudouridine (Ψ) and methods for providing a modified tRNA in the cell have been described above.

In some embodiments, the PTC is UAG and wherein the modified tRNA is tRNA-Lys, having an anticodon sequence of CUΨ.

In some embodiments, the PTC is UAA and wherein the modified tRNA is tRNA-Lys having an anticodon sequence of UUΨ.

In some embodiments, the PTC is UGA and wherein the modified tRNA is tRNA-Arg having an anticodon sequence of UCΨ.

In certain embodiments, the modified tRNA is an artificially synthetic tRNA.

In certain embodiments, the modified tRNA further comprises one or more modifications in one or more nucleotides of the modified tRNA, wherein the one or more modifications comprise pseudouridylation, 2′-O-methyl modification of sugar moiety, 2′-O-methoxyethyl modification of sugar moiety, phosphorothioate internucleoside linkage, phosphodiester internucleoside linkage, or a combination thereof. In certain embodiments, the one or more further modifications are at positions other than position 36 (N36).

### IV. Method for Expanding a Genetic Codon

Another aspect of the application relates to a method of expanding a genetic codon based on Ψ-Ψ pair decoding. The method comprises the step of a) modifying an original genetic codon (N1N2N3) that contains one or more uridines (U) in a mRNA in a cell by converting the one or more U residue(s) in the genetic codon into pseudouridine (Ψ) to create a modified genetic codon; b) providing a modified tRNA in the cell, wherein the modified tRNA comprises an anticodon that (1) contains one or more pseudouridines (Ψ) that pair with the one or more pseudouridines (Ψ) in the modified genetic codon, and (2) complements to the modified genetic codon at positions other than the one or more pseudouridines (Ψ), wherein the modified tRNA is capable of pairing with the modified genetic codon in the mRNA and incorporates an amino acid that is different from the amino acid encoded by the original genetic codon into a peptide encoded by the mRNA.

Table 1 herein below an example of expansion of the current genetic code based on the Ψ-Ψ pair decoding in S. cerevisiae.

### V. mRNA/tRNA Complex

Another aspect of the application relates to a mRNA/tRNA complex, comprising: a modified mRNA comprising a modified premature translation termination codon N1N2N3 (PTC), wherein the uridine at position N1 of the PTC has been converted to a pseudouridine (Ψ); and a modified tRNA, wherein the tRNA comprises a modified uridine at position N36 of the anticodon sequence N34N35N36 of the tRNA, wherein the pseudouridine (Ψ) in the modified mRNA pairs with the modified uridine in the anticodon sequence of the modified tRNA.

In certain embodiments, the modified uridine in the modified tRNA is pseudouridine (Ψ).

In certain embodiments, the modified PTC is PAG and wherein the modified tRNA is tRNA-Lys having a modified anticodon of CUP.

In certain embodiments, the modified PTC is ΨAA and wherein the modified tRNA is tRNA-Lys having a modified anticodon of UUP.

In certain embodiments, the modified PTC is ΨGA and wherein the modified tRNA is tRNA-Arg having a modified anticodon of UCΨ.

In certain embodiments, the modified tRNA further comprises one or more modifications in one or more nucleotides of the modified tRNA, wherein the one or more nucleotides comprise a 2′-O-methyl modified sugar moiety, a 2′-O-methoxyethyl modified sugar moiety, a phosphorothioate internucleoside linkage, or a phosphodiester internucleoside linkage, or a combination thereof. In certain embodiments, the one or more further modifications are at positions other than position 36 (N36).

### VI. Kits

Another aspect of the application is a kit for treating a PTC-related disease by nonsense suppression, the kit comprising: (1) a modified tRNA comprising a modified uridine at position 36 (N36) of an anticodon sequence N34N35N36 of the tRNA; and (2) a nucleic acid molecule for pseudouridylation of a target uridine in a target mRNA in a cell, wherein the nucleic acid molecule comprises a guide region capable of forming a partially double stranded nucleic acid complex with the target mRNA comprising the target uridine, wherein the partially double stranded nucleic acid complex is capable of engaging a mammalian pseudouridylation enzyme, wherein the guide region assists in positioning the target uridine in the partially double stranded nucleic acid complex for it to be converted to a pseudouridine by the mammalian pseudouridylation enzyme, wherein the target uridine is a uridine in a PTC; (3) instructions for using the kit.

Another aspect of the present application relates to a kit for treating a PTC-related disease by nonsense suppression, the kit comprising: (1) a tRNA targeting nucleic acid molecule for pseudouridylation of a target uridine in a target tRNA, wherein the tRNA targeting nucleic acid molecule comprises a guide region capable of forming a partially double stranded nucleic acid complex with the target tRNA in a cell, wherein the partially double stranded nucleic acid complex is capable of engaging a mammalian pseudouridylation enzyme, wherein the guide region assists in positioning the uridine in the tRNA in the partially double stranded nucleic acid complex for it to be converted to a pseudouridine by the mammalian pseudouridylation enzyme; and (2) a nucleic acid molecule for pseudouridylation of a target uridine in a target mRNA in a cell, wherein the nucleic acid molecule comprises a guide region capable of forming a partially double stranded nucleic acid complex with the target mRNA comprising the target uridine, wherein the partially double stranded nucleic acid complex is capable of engaging a mammalian pseudouridylation enzyme, wherein the guide region assists in positioning the target uridine in the partially double stranded nucleic acid complex for it to be converted to a pseudouridine by the mammalian pseudouridylation enzyme, wherein the target uridine is a uridine in a PTC; (3) instructions for using the kit.

### VI. Pharmaceutical Compositions

Another aspect of the present application relates to a pharmaceutical composition comprising (1) a nucleic acid molecule according to the present application, or an expression vector according to the present application, and (2) a pharmaceutically acceptable carrier.

Suitable pharmaceutically acceptable carriers are well known to the person skilled in the art.

In some embodiments, the pharmaceutically acceptable carrier is a saline solution. This can usefully be isotonic or hypotonic, particularly for pulmonary delivery. In some embodiments, the application also provides a delivery device (e.g. syringe, inhaler, nebulizer) which includes a pharmaceutical composition of the application.

The present application is further illustrated by the following examples that should not be construed as limiting. The contents of all references, patents, and published patent applications cited throughout this application, as well as the Figures and Tables, are incorporated herein by reference.

### EXAMPLES

### Methods

### Strains and Cell Culture

Yeast S. cerevisiae strains used in this work were YCL46 (MATa cup1Δ::ura3, leu2-3,112, ura3-52, trp1Δ63, lys2-801amber, ade2-101ochre, his3A200, GAL+) and BY4741 (MATa his3Δ1, leu2Δ0, met15Δ0, ura3Δ0). Cells were cultured in yeast extract-peptone-dextrose (YPD) complete medium for routine cultivation or synthetic dextrose (SD) medium for selection.

Human HEK293T cells were also used in this work and were cultured in DMEM high glucose supplemented with 10% fetal bovine serum. Subculture of HEK293T was performed when the cell confluency reached to 90-100%, and the PBS plus 1 mM EDTA solution was used.

The Escherichia coli strain XL10-Gold was used for cloning and was cultivated in LB broth.

### Oligonucleotides

Oligonucleotides sctKuuu_40-57R [SEQ ID NO:24, for yeast tRNALys(UUU)], sctKcuu_40-57R [SEQ ID NO:25, for yeast tRNALys(CUU)], and sctKuuu_39-56R [SEQ ID NO:26, for yeast tRNAArg(UCU)] were used as yeast tRNA probes in Northern blot analysis. As a control, sctLcaa_51-71R (SEQ ID NO:27) was used to probe yeast tRNALeu(CAA)65.

Oligonucleotide scSNR81_35-53R (SEQ ID NO:28) was used as the yeast gRNA probe in Northern blot analysis.

Oligonucleotides HtKuuu_41-62R [SEQ ID NO:29, for human tRNALys(UUU)] and HtKcuu_41-62R [SEQ ID NO:230, for human tRNALys(CUU)] were used as human tRNA probes in Northern blot analysis. As a control, the oligonucleotide HtSgcu_47-66R (SEQ ID NO:31) was used to probe human tRNASer(GCU).

Oligonucleotide CUP1_133-152R (SEQ ID NO:39) was used for primer-extension experiment (See Section “Pseudouridylation Assay”) to detect CUP1 mRNA pseudouridylation.

Oligonucleotides sctKuuu_40-57R [SEQ ID NO:24, for yeast tRNALys(UUU)], sctKcuu_40-57R [SEQ ID NO:25, for yeast tRNALys(CUU)], and sctKuuu_39-56R [SEQ ID NO:26, for yeast tRNAArg(UCU)] were used for primer-extension experiment to detect yeast tRNA pseudouridylation.

Chimeric oligonucleotides sctKuuu-U36rH [SEQ ID NO:32, for yeast tRNALys(UUU)], sctKcuu-U36rH [SEQ ID NO:33, for yeast tRNALys(CUU)], and sctRucu-U36rH [SEQ ID NO:34, for yeast tRNAArg(UCU)] were used to direct site-specific RNase H cleavage (See Section “Thin-layer chromatography”).

Oligonucleotide sets CUP1_50-69F (SEQ ID NO:38) and CUP1_133-152R (SEQ ID NO:39, for yeast CUP1 gene), mCherry_201-220F (SEQ ID NO:40) and mCherry_302-321R (SEQ ID NO:41, for mCherry gene), and EGFP_466-485F (SEQ ID NO:42) and EGFP_542-561R (SEQ ID NO:43, for EGFP gene) were used as qPCR primer sets in RT-qPCR experiments.

As controls, the oligonucleotide set TDH3_151-170F (SEQ ID NO:44) and TDH3_254-273R (SEQ ID NO:45) was used to amplify yeast reference gene TDH3, and the set hACTB_744-765F (SEQ ID NO:46) and hACTB_941-962R (SEQ ID NO:47) for human reference gene ACTB.

### Plasmid

Yeast gRNA genes were amplified through primer overlapping PCR. Genes were inserted into the YEplac181-pTDH3-3×3′ETS plasmid [LEU2, 2-micron] between SalI and SacI, between KpnI and XhoI, between NheI and PstI, and/or in BamHI sites.

Yeast reporter cassettes were constructed as follows: TDH3 promoter was inserted into the YEplac195 plasmid [URA3, 2-micron] between EcoRI and BamHI sites, RPS25A intron between BamHI and SalI, 3×FLAG tag with Kozak sequence between SacI and KpnI, and DIT1 terminator between SalI and HindIII. The wild-type yeast CUP1 or mCherry gene was inserted into the cassette between KpnI and SalI sites to form YEplac195-CUP1 or YEplac195-mCherry plasmid, respectively. Mutant gene constructs with PTC were obtained through site-directed mutagenesis. All the plasmids were transformed into yeast through standard lithium acetate transformation.

Human reporter cassettes were constructed as follows: Wild-type and mutant 3×FLAG-mCherry fusion protein genes were cloned and inserted into the pcDNA3.1-Zeo(+) plasmid between NheI and XbaI sites to form pcDNA-mCherry plasmids. The BleoR antibiotic-resistant protein gene was replaced with EGFP gene through Gibson Assembly® (NEB) to form pcDNA(EGFP)-mCherry plasmids.

Human gRNA expression cassettes were constructed as follows: human U6 promoter-EcoRI-HindIII-XbaI-KpnI-polyT terminator cassette was inserted into the pEGFP-N1 plasmid through Gibson Assembly® (NEB), and the CMV immediate-early enhancer and promoter were substituted with the human PGK1 promoter (SnapGene) to form pU6E plasmid. The PTC-gRNA genes were inserted between EcoRI and HindIII sites, and the tRNA-gRNA genes were between XbaI and KpnI. A non-targeting shRNA (5′-CAACAAGATGAAGAGCACCAA-3′, Sigma-Aldrich) was inserted between HindIII and XbaI sites. This shRNA gene serves as an interval between two gRNAs, which will be cut by DROSHA. To construct the plasmids for lentiviral transduction, the cassettes from the pU6E plasmids were cloned and substituted for the internal U6 promoter of pLKO.5-puro (Sigma-Aldrich) plasmid through Gibson Assembly® (NEB) to form pLKO.5-gRNA plasmids. All the plasmids were delivered into HEK293T cells with jetPRIME® transfection reagent (Polyplus), according to the manufacturer's instructions.

### RNA Extraction

Yeast and human total RNA were extracted with TRIzol™. Yeast cells were first suspended in TRIzol™ and disrupted four times by bead-beating. RNA extraction was then performed according to the manufacturer's instructions. Human total RNA extraction was carried out directly following the manufacturer's instructions.

To directly extract the yeast total tRNA, we followed the previously described protocol. Briefly, yeast cells were first suspended in three times the volume of Extraction Buffer (0.1 M sodium acetate, pH 5.0 and 0.1% (w/V) SDS), mixed with twice the volume of phenol [saturated with 0.1 M citrate buffer, pH 4.3 (Sigma-Aldrich)], and vigorously shaken on a thermoshaker at room temperature for 2 hours. The mixture was then centrifuged at 13,000 g for 15 min, and the supernatant was then extracted once with an equal volume of acid phenol-chloroform-isoamyl alcohol (25:24:1). The RNA was then precipitated with three times the volume of ethanol, washed once with 10 mM acetic acid in 75% (V/V) ethanol, and dissolved in RNase-free ddH2O.

To preserve tRNA charging during extraction, the experiments described above were performed on ice or at 4° C. The RNA pellet (either total RNA or tRNA) was dissolved in ice-chilled 10 mM sodium acetate, pH 5.0, and directly used for downstream experiments.

### Pseudouridylation Assay

Pseudouridylation assay was performed as previously described66. Briefly, about 10 μg total RNA or 5 μg total tRNA was first treated with 200 mM CMCT in BEU buffer (50 mM bicine-NaOH, pH 9.0, 7 M urea, and 4 mM EDTA) and washed with 100 mM sodium carbonate plus 1 mM EDTA to remove CMCT adducts from G, I and unmodified U nucleotides. RNA then served as templates for reverse transcription/primer extension with 32P-radiolabeled primers. The primer-extension products were then resolved on a 7%-urea polyacrylamide gel, exposed to a phosphor screen, and visualized by autoradiography (Typhoon RGB, Cytiva). The product bands were recognized by Image Lab software (Bio-Rad) and quantified.

### Thin-Layer Chromatography

About 5-10 μg yeast tRNALys(UUU), tRNALys(CUU), and tRNAArg(UCU) were first isolated from 500 μg yeast total tRNA by biotinylated anti-sense tRNA oligonucleotides bound to Pierce™ High Capacity Streptavidin Agarose (Thermo Scientific). 1 μg isolated tRNA was then site-specifically cleaved at the 5′ side of U36 by RNase H (Sigma) directed by 2′-O-methyl RNA-DNA chimeric oligonucleotides. The 3′ cleaved fragment was then dephosphorylated by FastAP (Thermo Scientific) and radiolabeled with [γ-32P]-ATP (PerkinElmer) by T4 PNK (Thermo Scientific). The radiolabeled tRNA fragment was further purified with a 12% urea polyacrylamide gel, digested into nucleoside 5′-monophosphates by Nuclease P1 (NEB), and developed in Cellulose F TLC plates (Sigma-Aldrich) with solvent C [isopropanol:concentrated HCl:water=68:18:14 (V:V:V)]. TLC plates were exposed to a phosphor screen and were visualized by autoradiography (Typhoon RGB, Cytiva). The nucleotide spots were recognized by Image Lab software (Bio-Rad), and the quantification was performed.

For CUP1 mRNA, the 3 mg total RNA was used for site-specific cleavage (cleaved immediately 5′ of the PTC and the normal stop codon) by the DNAzyme 10-23: scCUP1K30UAA_10-23 and scCUP1stop_10-23. The cleaved 3′ mRNA fragment (beginning with the PTC, which was targeted by a PTC-specific or PTC-unspecific gRNA) was then hybridized with a biotinylated mRNA probe, pulled down by Pierce™ High Capacity Streptavidin Agarose, radiolabeled with [γ-32P]-ATP by T4 PNK, digested with nuclease P1 to completion, and developed as described above.

### Northern Blot Analysis

5 μg of total RNA were resolved on a 7% urea polyacrylamide gel and transferred onto the Amersham Hybond-N+ membrane (Cytiva) in 0.5×TBE with 10V overnight at 4° C. RNA on the membrane was then crosslinked with 254 nm UV light, prehybridized with 100 μg/mL carrier RNA in hybridization buffer (6×SSC in 50 mM sodium phosphate, pH 6.5 and 0.5% (w/V) SDS) at 42° C. for 2 hours followed by hybridization with 32P-radiolabeled tRNA or gRNA probes at 42° C. overnight. The membrane was then washed four times with wash buffer (1×SSC and 0.1% (w/V) SDS), exposed to a phosphor screen, and visualized by autoradiography (Typhoon RGB, Cytiva). The RNA bands were recognized by Image Lab software (Bio-Rad), and the quantification was performed.

For the charged tRNA, the experiments were carried out as described above, except that 5 μg of total tRNA (yeast) or total RNA (human) were resolved on a 7% acid urea polyacrylamide gel and transferred onto the Amersham Hybond-N+ membrane (Cytiva) in 100 mM Tris-HCl, pH 8.0 with 10V overnight at 4° C.

### Copper Sensitivity Assay

Copper sensitivity assay was performed. Briefly, plates with selective medium were prepared by mixing the warm SD medium with copper sulfate before the medium congealed. Mid-log phased yeast cells were first suspended in autoclaved water to a final OD600 of 0.2, 0.02, and 0.002, and spread on plates in droplets of 7.5 L. The plates were then incubated at 30° C. for 48 hours, and the images were captured by Gel Doc XR+ Imaging System (Bio-Rad) with the exposure time fixed at Is.

### Western Blot Analysis

Mid-log phased yeast cells were collected and suspended in RIPA buffer (Thermo Scientific) supplemented with 200 μM PMSF, 1 g/mL pepstatin, and 10 mM 2-mercaptoethanol.

Cells were then disrupted and lysed four times by bead-beating, and the supernatant was collected and mixed with NuPAGE™ LDS Sample Buffer (Invitrogen) after centrifugation. Proteins were then resolved through SDS-PAGE and transferred to Immun-Blot® Low Fluorescence PVDF Membrane (Bio-Rad) by Trans-Blot Turbo Transfer System (Bio-Rad). The membranes were then blocked by 5% (w/V) non-fat milk solution, probed with primary antibodies and Alexa Fluor conjugated secondary antibodies, and visualized by fluorescence imaging (Typhoon RGB, Cytiva). Bands of the proteins were recognized by Image Lab software (Bio-Rad), and the quantification was performed.

For human cells, the experiments were carried out as described above, except that cells were first detached and collected in PBS plus 1 mM EDTA, pelleted by centrifugation, resuspended in resuspension buffer (TBS supplemented with 200 M PMSF, 1 g/mL pepstatin, and 10 mM 2-mercaptoethanol), and lysed by adding equal volume of RIPA buffer and pipet-mixing.

### RT-qPCR

About 1 μg total RNA was used as a template for reverse transcription as described above, except that the primer here was Oligo_dT-18. The cDNA (RT product) solution was then diluted to 100 μL with dilution buffer (10 mM Tris plus 1 mM EDTA). For each qPCR reaction, 1 μL cDNA dilution was mixed with 5 μL SYBR™ Select Master Mix (Applied Biosystems), 400 M qPCR primer sets, and ddH2O to 10 μL. qPCR reactions were performed with QuantStudio™ 5 (Applied Biosystems) in standard mode, and the comparative Ct (ΔΔCt) was calculated.

### Red Fluorescence Imaging and Data Quantification

Red fluorescence images were acquired from ECLIPSE Ts2-FL inverted microscope (Nikon), and the data were then analyzed by NIS-Elements BR. Briefly, the yeast colonies were identified via bright-field imaging and used for in situ red fluorescent signal quantification. The obtained mean intensity was then normalized to the exposure time to yield “Intensity per millisecond”. All quantifications were carried out in at least three independent biological replicates.

### Optical Melting Measurements

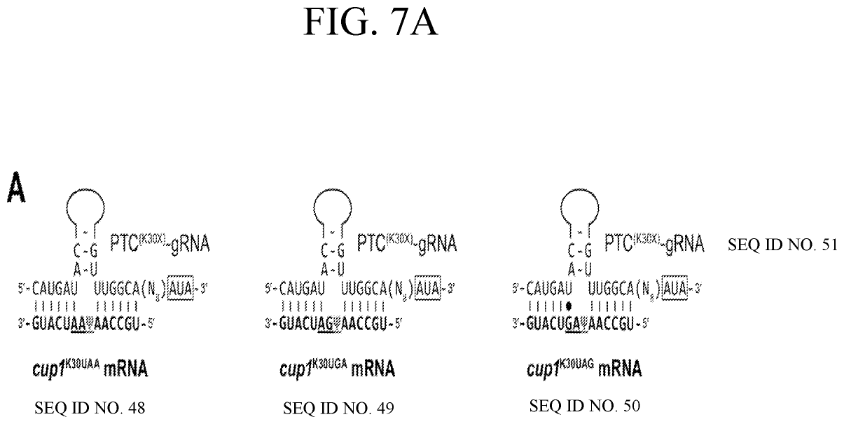

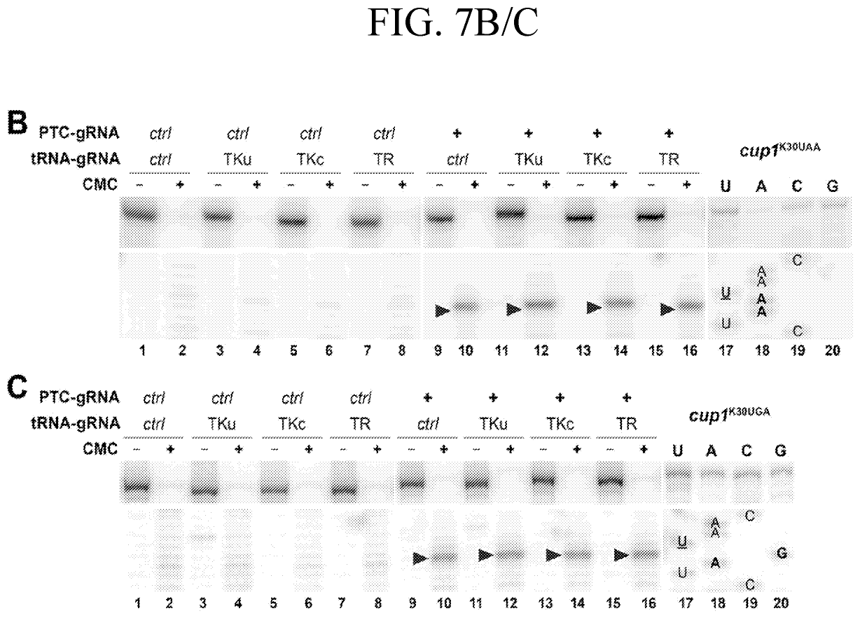

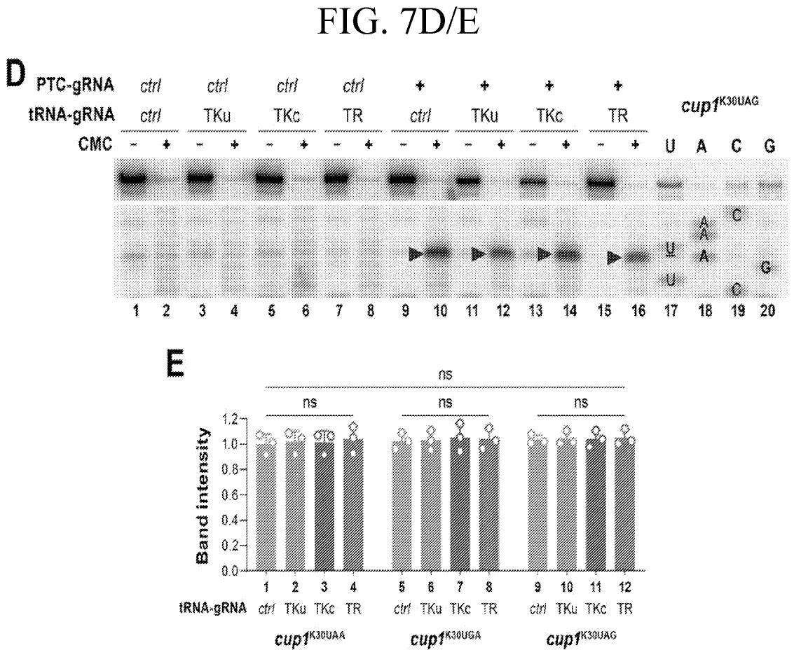

The optical melting experiments shown in FIG. 7 were performed in a phosphate buffer. RNA oligonucleotides were first dissolved in melting buffer (20 mM sodium phosphate, pH 7.0, 980 mM sodium chloride, and 1 mM EDTA) in a concentration of ~1 mM. Oligos were then mixed, diluted to 2, 5, or 10 μM concentration, and pre-annealed in the melting buffer by denaturing the oligo mix under 95° C. for 2 min, and gradually cooling down to room temperature with the ramp rate of −0.1° C./second.

These optical melting measurements were performed in Shimadzu UV-1800 spectrophotometer, and the temperature range was set from 10° C. to 90° C. The acquired OD260 absorbance versus temperature data were then analyzed by MeltR69 using “global fitting” to determine the concentrations and to calculate the thermodynamic parameters, which were represented with mean±standard error.

Optical melting experiments for determining motif stabilities were studied in cacodylate buffer to facilitate comparison to prior experiments35. Measurements were performed for nine concentrations for the RNA duplexes between the ranges of 0.1 mM and 1 μM in buffer containing 1 M sodium chloride, 20 mM sodium cacodylate, and 0.5 mM Na2EDTA at pH 7.

Absorbance vs. temperature melting curves were measured at 260 nm with a heating rate of 1° C./min from 0 to 90° C. on JASCO V-650 UV/Vis spectrophotometer with a thermoprogrammer. The melting curves were analyzed using a two-state model with MeltWin 3.570. For all optical melting experiments, single strand oligonucleotide concentrations were calculated from the absorbance above 80° C. and single strand extinction coefficients were approximated by a nearest-neighbor model.

### Lentiviral Transduction

HEK293T cells were first cultivated in 100 mm culture dish to 75-85% confluency and transfected with pLKO.5-gRNA, pMD2.G, pMDLg-pRRE, and pRSV-Rev plasmids of 3.5, 2, 3, and 1.5 μg, respectively, for lentiviral production. The culture medium was replaced 24 hours post-transfection and collected 60 hours post-transfection. The collected medium with lentiviral particles was then directly used for the transduction of freshly cultured HEK293T cells, and the cells were then subjected to the selection of 3 μg/mL puromycin (Gibco) twice to generate stable cell lines.

### Data Processing and Analysis

Data from pseudouridylation assay, thin-layer chromatography, northern blot analysis, western blot analysis, RT-qPCR and red fluorescence quantification were processed and analyzed in GraphPad Prism 10 with ANOVA with post hoc tests. Error bars represent the estimated mean±standard deviation. All quantifications were done in at least three independent biological replicates and in three technical repeats for each biological replicate.

### Example 1. Designing the mRNA Targeting Nucleic Acid Molecules

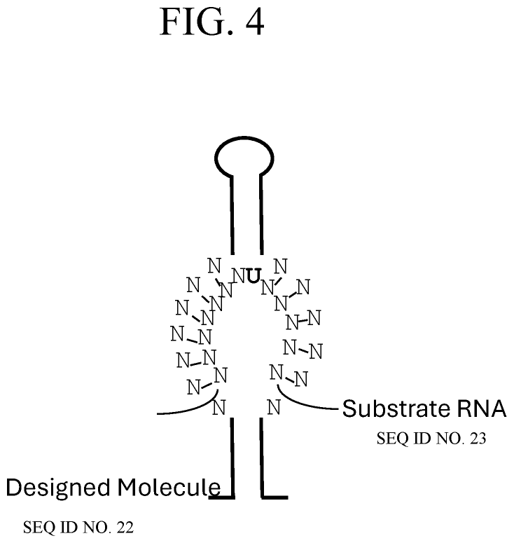

Based on the mRNA target sequences, the guide sequences within the guiding pockets of a box H/ACA RNA are designed to be complementary to the targeted sequences (FIG. 4). The designed molecule should be able to base pair with the mRNA target as followed but not limited to:

| | | |
|---|---|---|
| Designed molecule: | 5′- NNNNN**NNNNN . . .-3′ | (SEQ ID NO: 22) |
| | \|\|\|\|\| \|\|\|\|\| | |
| mRNA substrate: | 3′-. . . NNNNNUNNNNNN . . .-5′ | SEQ ID NO: 23) |

The targeted uridine for pseudouridylation is in bold. The “*” stands for the upper stem-loop structure that usually stays unchanged unless necessary. 2D structure prediction is performed after the designing by using the software such as but not limited to RNAfold, RNAStructure, etc. to examine if the molecule could fold into an unexpected structure (i.e., not the canonical hairpin-hinge (box H)-hairpin-tail (box ACA) structure) and if this unexpected structure is dominant. Should it occur, sequences outside the guiding pockets would also be modified to correct the structure.

### Example 2. Designing the tRNA Targeting Nucleic Acid Molecules

Based on the tRNA target sequences, the guide sequences within the guiding pockets of a box H/ACA RNA are designed to be complementary to the targeted sequences (FIG. 4). The designed molecule should be able to base pair with the tRNA target as followed but not limited to:

| | | |
|---|---|---|
| Designed molecule: | 5′- NNNNN**NNNNN . . .-3′ | (SEQ ID NO: 22) |
| tRNA substrate: | 3′-. . . NNNNNUNNNNNN . . .-5′ | SEQ ID NO: 23) |

The targeted uridine for pseudouridylation is in bold. The “*” stands for the upper stem-loop structure that usually stays unchanged unless necessary. 2D structure prediction is performed after the designing by using the software such as but not limited to RNAfold, RNAStructure, etc. to examine if the molecule could fold into an unexpected structure (i.e., not the canonical hairpin-hinge (box H)-hairpin-tail (box ACA) structure) and if this unexpected structure is dominant. Should it occur, sequences outside the guiding pockets would also be modified to correct the structure.

### Example 3: Cloning Both the tRNA and the mRNA Targeting Molecules into Tandem Expression Cassettes

Sequence of the plasmid vector for S. cerevisiae tRNA and mRNA is shown in SEQ ID NO:20.

### Cloning Steps:

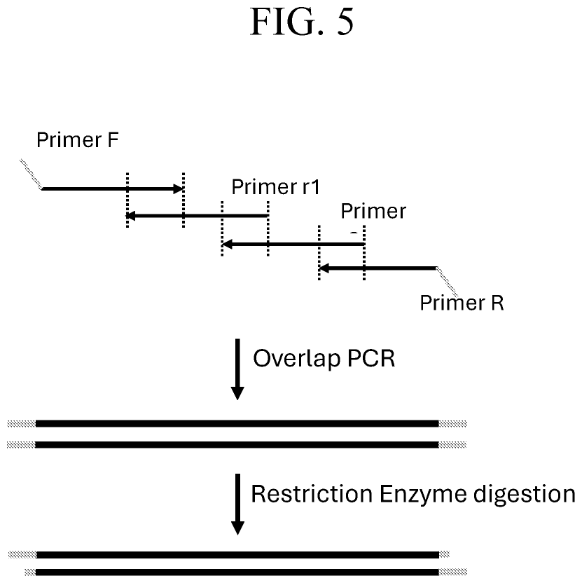

tRNA and mRNA targeting molecules were amplified using overlap PCR (FIG. 5). One forward primer (primer F) and multiple reverse primers (primer R, primer r1, etc.) are designed. The 5′ end of the primer F and primer R possesses the restriction enzyme recognition sequences (for tRNA targeting molecules, XbaI and PstI; for mRNA targeting ones, SalI and SacI). The 3′ end of the primer F anneals to the 3′ end of the primer r1 and all the reverse primers overlap with each other tandemly. The primers were mixed in 20 pmol:1 pmol: . . . :20 pmol ration (primer F to primer r1 to . . . to primer R), diluted in 25 μL double distilled water (ddH2O), and mixed with 25 μL Q5® High-Fidelity 2× Master Mix. PCR amplification program was set as followed: 30 second at 95 degree Celsius for pre-denaturing, 35 cycles of 10 second at 95 degree Celsius for denaturing−20 second at 50 degree Celsius for annealing−10 second at 72 degree Celsius for elongating, 3 min at 72 degree Celsius for post-elongating and cooling at 12 degree Celsius. The amplified molecules were then separated with 2% agarose gel electrophoresis with 0.5×TBE buffer. Gel slices with the amplified molecules were excised and gel-extracted using QIAquick Gel Extraction Kit (QIAGEN). Purified molecules were then digested with restriction enzymes (for tRNA targeting molecules, FastDigest XbaI and FastDigest PstI; for mRNA targeting ones, FastDigest SalI and FastDigest SacI, ThermoFisher). At the meantime, the plasmid vectors were also digested with the restriction enzymes (for tRNA targeting molecules, FastDigest NheI and FastDigest PstI; for mRNA targeting ones, FastDigest SalI and FastDigest SacI; ThermoFisher) and FastAP alkaline phosphatase (ThermoFisher). The digested molecules and vectors were purified using QIAquick PCR Purification Kit (QIAGEN). Purified digested molecules and vectors were mixed in a molar ratio of 10:1 (molecules to vectors) and ligated using T4 DNA ligase (Thermofisher). The ligation mixture mere used for subsequent Escherichia coli transformation and spread onto the plates with ampicillin for selection. The colonies were picked up for small-scale liquid culturing and the plasmids were extracted using QIAprep Spin Miniprep Kit (QIAGEN).

Sequence of the plasmid vector for H. sapiens tRNA and mRNA is shown in SEQ ID NO:21.

### Example 4: Introducing the Expression Cassettes into Cells, Either Via Transformation (Saccharomyces cerevisiae) or Transfection (Homo sapiens)

S. cerevisiae: Plasmid Transformation

Early log-phased cells of 0.8 OD600 unit amount were first collected by centrifuge at 3000 g for 5 min, washed with 1 mL 100 mM lithium acetate once, and then incubated with 300 ng plasmid vector with the report gene and 300 ng plasmid vector with the mRNA and tRNA targeting plasmid vector in 100 μL OST buffer (100 mM lithium acetate, 100 mM DTT, 40% (w/V) PEG-3350) for 30 min at 42° C. After incubation the cells were collected by centrifuge at 3000 g for 5 min and spread onto the Synthetic Dextrose medium (leucine and uracil double dropout). The colonies were picked up for downstream experiments.

H. sapiens Tissue: Plasmid Transfection

Transfection was generally performed according to the vender's protocol of jetPRIME® transfection reagent (Polyplus). Adhesive HEK293T cells (from ATCC) were first cultured 24 h in DMEM medium (high glucose, with L-glutamine, without sodium pyruvate; Gibco) supplemented with 10% (V/V) fetal bovine serum (Corning) in 6-well plates with 40% confluency before transfection. 0.75 μg plasmid vector with the report gene and 0.75 μg plasmid vector with the mRNA and tRNA targeting plasmid vector were mixed and diluted in 150 L jetPRIME® buffer. Mixture was then vortexed for 10 second, and 3 μL jetPRIMIE® reagent was added. After 10 second vortexing, the mixture was added dropwise into the cells. The medium was replaced 24 hours post-transfection and the cells were harvested 72 hours post-transfection for downstream experiments.

### Example 5: Detection of the Full Length Translation Product

Cell lysate acquisition. For S. cerevisiae, late log-phased cells of 30 OD600 unit amount were collected by centrifuge at 3000 g for 5 min, washed once with 5 mL (ddH2O), resuspended in 300 μL RIPA lysis and extraction buffer (Thermofisher), and mixed with 100 g 0.5 mm diameter glass beads. Cells was then disrupted and lysed with bead beating four times.

For H. sapiens tissue, cells in 6-well plates 72 h post-transfection with 100% confluency were collected through treating with 1 mL 1 mM EDTA in phosphate buffered saline (PBS) followed by centrifuge at 300 g for 3 min. Cell pellet was resuspended in 75 μL Tris-buffered saline (TBS) and lysed by adding another 75 μL RIPA lysis and extraction buffer (Thermofisher).

Denaturing poly acrylamide electrophoresis. Cell lysate acquired above was then centrifuged at 16000 g for 10 min, and the suspension was mixed withNuPAGE™ LDS Sample Buffer (4×; Invitrogen). 10-15 μL mixture was loaded onto 10% bis-Tris SDS polyacrylamide gel. Electrophoresis was performed with Tris-MOPS-SDS buffer (50 mM Tris, 50 mM MOPS free acid, 0.1% (w/V) SDS, 1 mM EDTA) with constant 100V voltage until the loading dye reached to the bottom.

Protein transferring and blotting. Proteins were then transferred to Amersham nitrocellulose Western blotting membranes (0.22 m pore size; cytiva) using Trans-Blot Turbo Transfer System (Bio-Rad) with maximum 23V voltage and 1.3 A current for 7 min. Membranes were then blocked with 10 mL 5% (w/V) fat-free milk in TBS plus 0.1% (w/V) Tween-20 (TBST; VWR) for one hour, washed once with 10 mL TBST for 5 min, incubated with 10 mL DYKDDDDK Tag monoclonal antibody (FG4R, 1:2000 dilution in 5% (w/V) bovine serum album (BSA) in TBST; Invitrogen), 10 mL GAPDH Loading Control monoclonal antibody (GA1R, 1:2000 dilution in 5% (w/V) BSA in TBST; Invitrogen), or alpha Tubulin monoclonal antibody (YL1/2, 1:1000 dilution in 5% (w/V) BSA in TBST; Invitrogen) overnight, washed three times with 10 mL TBST for 10 min, probed with Anti-mouse IgG, HRP-linked Antibody (1:5000 dilution in 5% (w/V) fat-free milk in TBST; Cell signaling Technology), washed twice with 10 mL TBST for 10 min, incubated with Radiance ECL (azure biosystems), and visualized by CCD camera (azure biosystems).

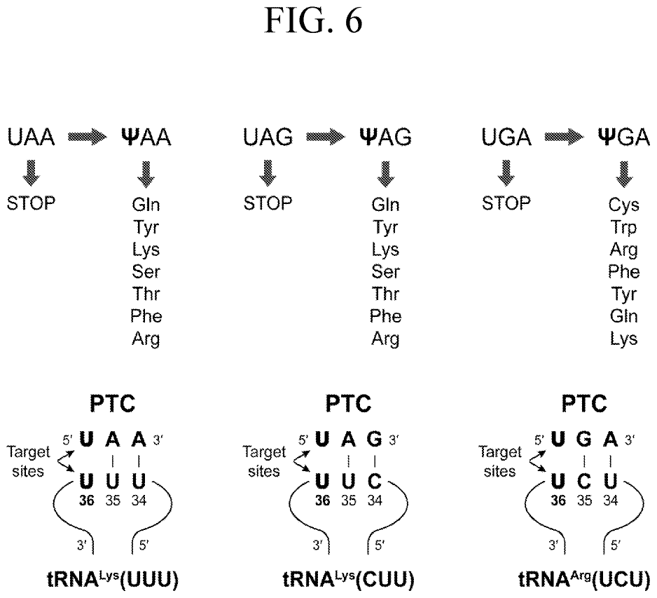

The Ψ-A pair is more stable than the U-A pair and that Ψ appears to be more flexible in pairing with other nucleotides. For the latter, Ψ is not only capable of pairing with A and G, but it can also pair with Uridine. This appears to be true in codon-anticodon recognition during protein translation. Through determining the amino acids incorporated at the pseudouridylated stop codons with mass spectrometry, several different tRNAs have been identified that can recognize the pseudouridylated stop codons (see FIG. 6): tRNA^{Ser}, tRNA^{Thr}, tRNA^{Gln}, tRNA^{Tyr}, tRNA^{Phe}, tRNA^{Lys}, and tRNA^{Arg} for the TAG and TAA codons; and tRNA^{Tyr}, tRNA^{Phe}, tRNA^{Trp}, tRNA^{Cyc}, tRNA^{Gln}, tRNA^{Lys}, and tRNA^{Arg} for the ΨGA codon. It is not surprising to identify multiple tRNAs for each pseudouridylated stop codon given that there are no cognate tRNAs for stop codons. Those tRNAs that have similar affinity to the stop codons would compete for recognition. The anticodons of some of these tRNAs appear to form a U-Ψ pair with the pseudouridylated stop codons. Specifically, tRNA^{Lys}(UUU) and tRNA^{Lys}(CUU) appear to pair with ΨAA and ΨAG, respectively. On the other hand, tRNA^{Arg}(UCU) is likely to pair with ΨGA. To further study the effect of P on codon-anticodon pairing, artificial box H/ACA RNAs were designed to target the uridines on either or both strands (the codon of mRNA and the anticodon of tRNA). The effect of pseudouridylation on translation could then be measured using the nonsense suppression systems, in which codon-anticodon pairing is reflected in nonsense codon read-through and can be readily quantified.

### Example 6: TRNA as Well as mRNA can be Targeted for Pseudouridylation by Designer Box H/ACA gRNAs

Designer box H/ACA gRNAs are capable of targeting mRNA pseudouridylation; however, it is not clear whether designer gRNAs can target tRNA for modification. Ample experimental results indicate that tRNA pseudouridylation is exclusively catalyzed by stand-alone protein pseudouridine synthases (PUSs) in eukaryotic cells. In other words, eukaryotic tRNA pseudouridylation is not naturally catalyzed by box H/ACA gRNA-guided mechanism. Thus, using the Saccharomyces cerevisiae system, the study first set out to determine if the designer box H/ACA gRNAs were able to direct not only PTC (mRNA) pseudouridylation but also tRNA pseudouridylation at the desired anticodon site.

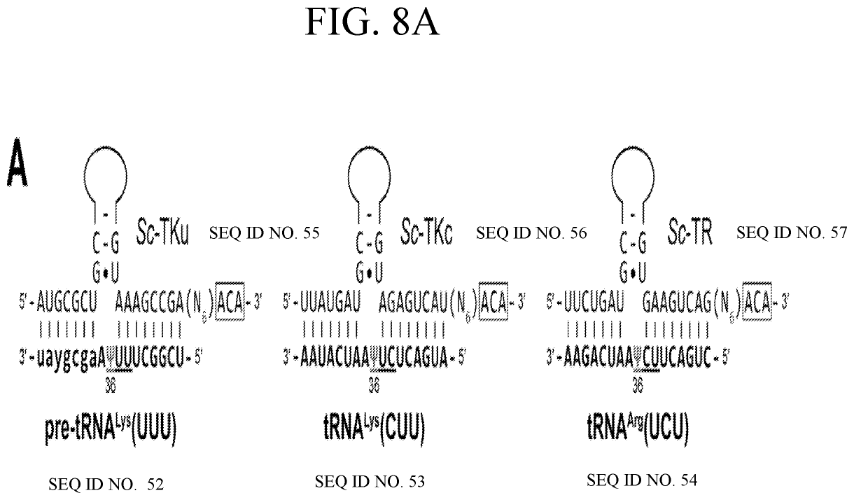

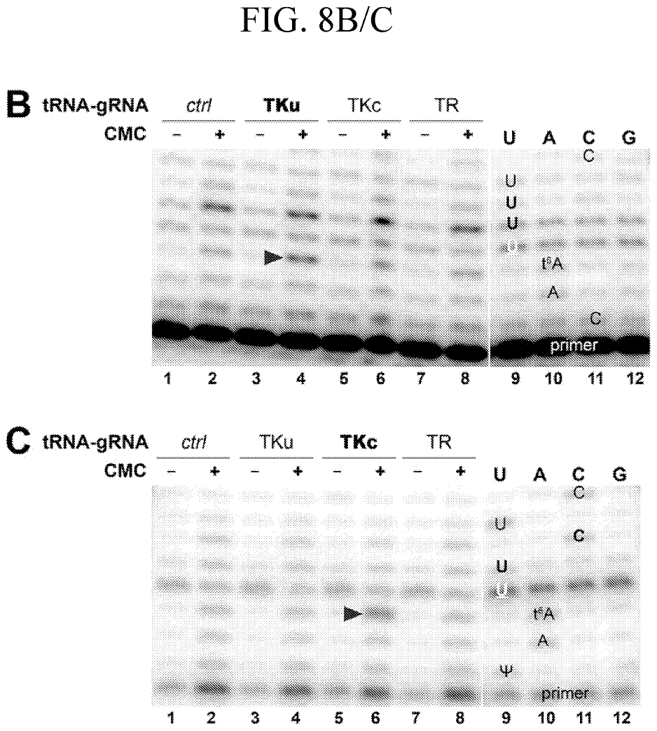

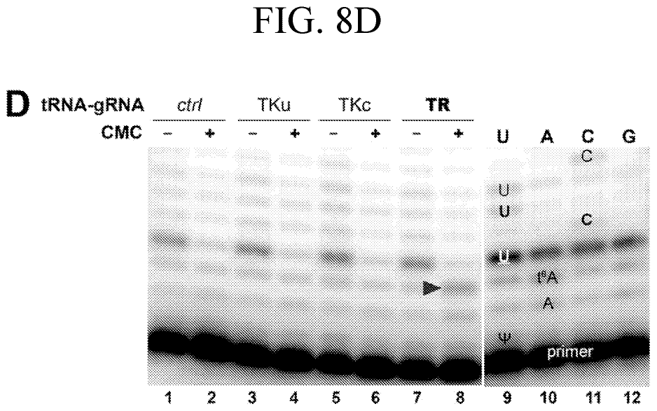

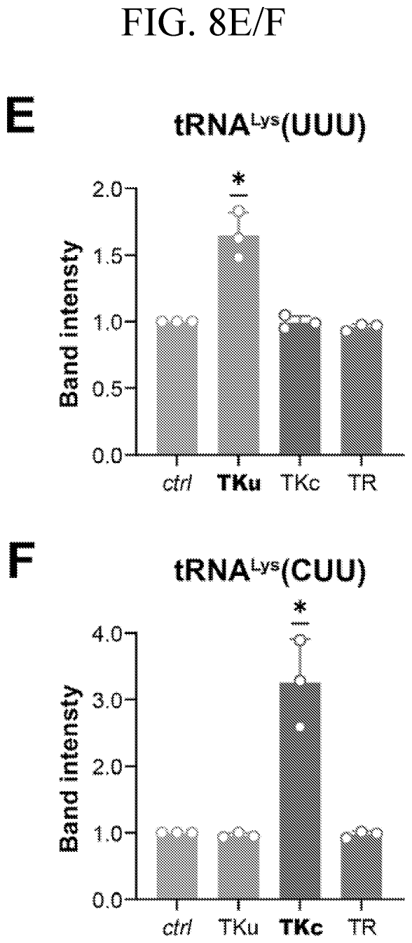

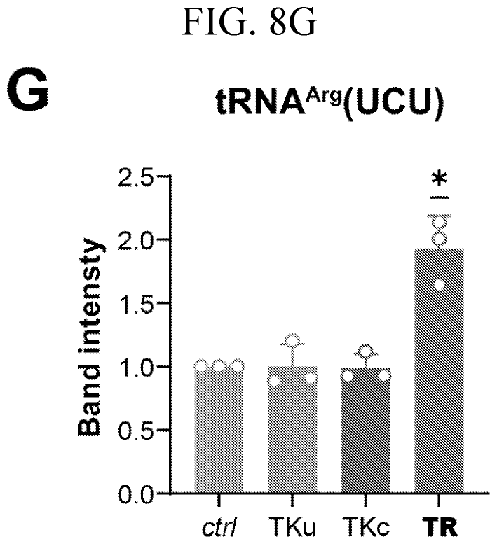

The study constructed a plasmid containing the cup1 reporter gene with a PTC at codon 30 (K30X), and another plasmid with a gRNA gene targeting the PTC of cup1 mRNA (FIG. 7A). The study also constructed another three plasmids each carrying a designer box H/ACA gRNA sequence targeting U₃₆ of tRNA^{Lys}(UUU₃₆), tRNA^{Lys}(CUU₃₆) or tRNA^{Arg}(UCU₃₆), respectively (FIG. 8A). The study then selectively transformed yeast cells with the PTC-containing cup1gene and the gRNA genes. After culturing, the study collected cells, recovered total cellular RNA, and checked the pseudouridylation of mRNA and relevant tRNAs at the target sites.

The study used a standard pseudouridylation assay, namely CMC-modification followed by alkaline hydrolysis and primer-extension, to detect Ψ in tRNA as well in mRNA. As shown in FIGS. 7B, 7C, and 7D, when cells were transformed with the PTC-containing cup1 gene and the PTC-specific gRNA gene or co-transformed with an additional tRNA-gRNA gene, the study detected a primer-extension stop/pause one nucleotide before the target pseudouridylation site at the mRNA PTC, indicating that the target site was indeed site-specifically pseudouridylated and that any other tRNA-gRNAs (co-transformed) showed no interference with this site-specific PTC pseudouridylation (lanes 10, 12, 14, and 16); no primer-extension stop/pause was detected when the PTC-specific gRNA gene was absent (lanes 2, 4, 6, and 8). Indeed, quantification analysis indicated that non-specific gRNAs had no effect on specific gRNA-directed pseudouridylation (FIG. 7E).

The study also detected site-specific pseudouridylation at the target site (Ψ₃₆) of tRNA^{Lys}(UUU₃₆), tRNA^{Lys}(CUU₃₆), and tRNA^{Arg}(UCU₃₆), when a target-specific designer tRNA-gRNA was introduced into the cells; transformation with non-specific tRNA-gRNAs did not lead to tRNA pseudouridylation at the desired target site (FIG. 8). Specifically, to target U₃₆ of tRNA^{Lys}(UUU₃₆), only transformation of tRNA^{Lys}(UUU₃₆)-gRNA resulted in the U₃₆-to-Ψ₃₆ conversion (FIG. 8B, lane 4); transformation of all the other gRNAs [including tRNA^{Lys}(CUU₃₆)-gRNA, tRNAArg(UCU₃₆)-gRNA]did not lead to tRNA^{Lys}(UUU₃₆) pseudouridylation at U₃₆ (FIG. 8B, lanes 6 and 8). Likewise, tRNA^{Lys}(CUU₃₆)-gRNA and tRNA^{Arg}(UCU₃₆)-gRNA specifically directed pseudouridylation of tRNA^{Lys}(CUU₃₆) and gRNA-tRNA^{Arg}(UCU₃₆), respectively, at U₃₆ (FIG. 8C, lane 6; FIG. 8D, lane 8); the other non-specific tRNA-gRNAs failed to guide the reaction at the target site (FIG. 8C, lanes 4 and 8; FIG. 8D, lanes 4 and 6). Quantitative analysis confirmed the results (FIGS. 8E, 8F, and 8G). The study concluded that pseudouridylation could also be re-directed to the new sites of tRNA by designer gRNAs in a highly site-specific manner.

### Example 7: Designer gRNA-Directed Pseudouridylation Occurs Less Efficiently in tRNA than in mRNA

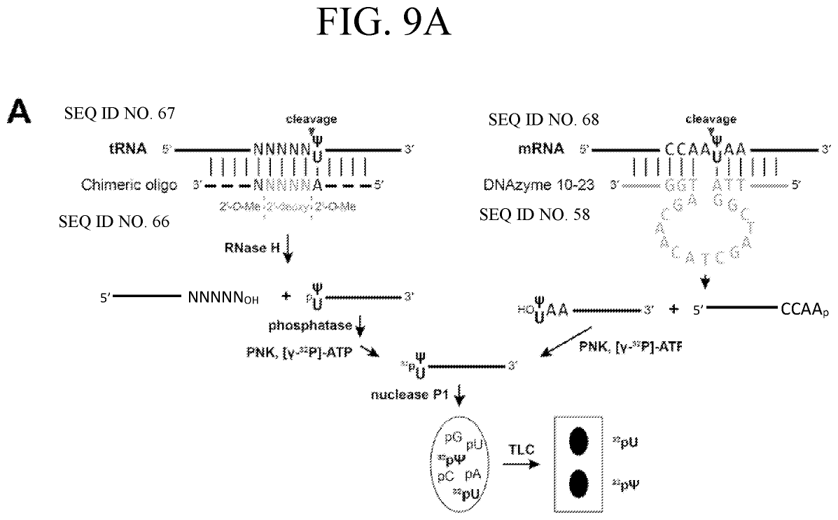

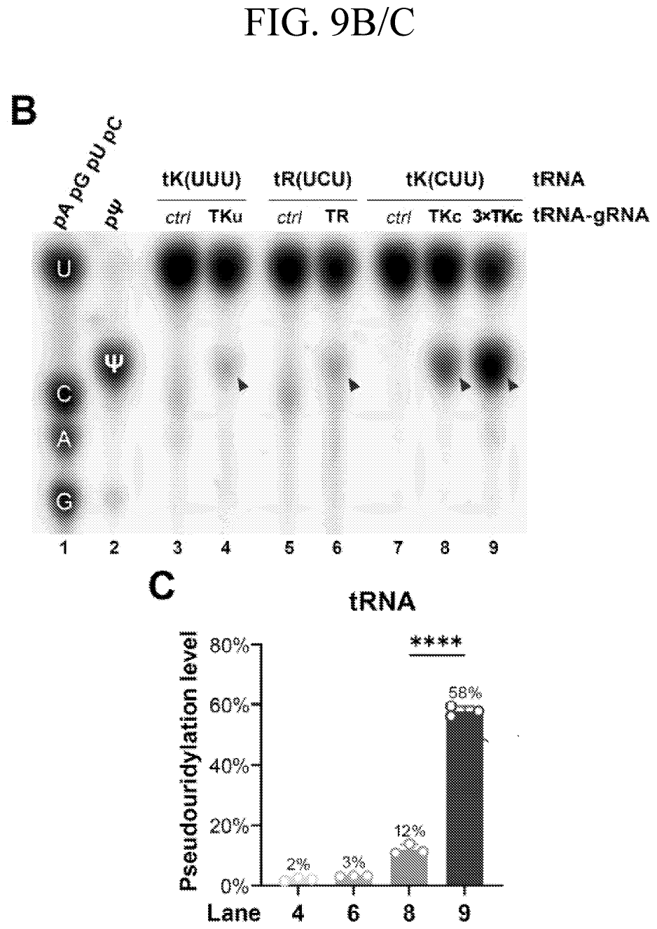

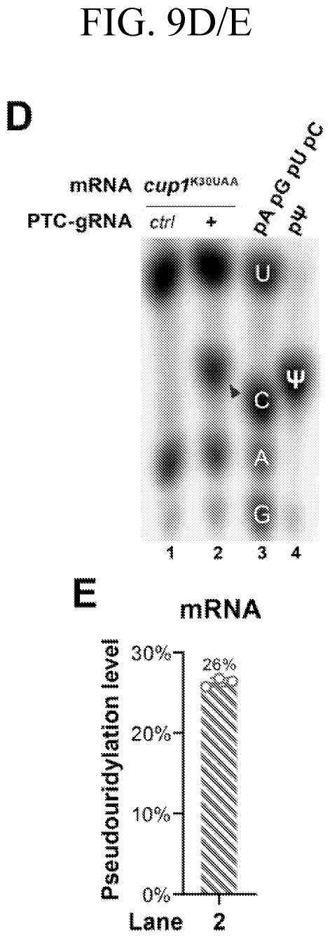

Although the study detected gRNA-guided tRNA pseudouridylation, it was not clear as to what extent the tRNAs were pseudouridylated. The study next carried out quantitative analysis using site-specific pseudouridylation measurement assay. tRNA^{Lys}(UUU), tRNA^{Lys}(CUU) or tRNA^{Arg}(UCU) was purified from cells transformed or untransformed with relevant tRNA-gRNAs. After specific cleavage 5′ of the target site, the target nucleotide (U, if not pseudouridylated; or Ψ, if pseudouridylated), located at the 5′ end of the digested 3′ fragment, was ³²P labeled with [γ-³²P]-ATP and polynucleotide kinase. Upon purification and nuclease P1 digestion of the ³²P-labeled fragment, ³²pU or ³²pΨ was then resolved on a TLC plate and quantified (FIG. 9A).

As shown in FIG. 9B, tRNA^{Lys}(UUU), tRNA^{Lys}(CUU) or tRNA^{Arg}(UCU) isolated from cells transformed with non-specific gRNAs showed no pseudouridylation at the target site—there was only a U spot and no Ψ spot was detected (lane 3, 5 and 7). In contrast, when tRNA^{Lys}(UUU), tRNA^{Lys}(CUU) or tRNA^{Arg}(UCU), purified from cells transformed with specific tRNA-gRNAs, was used, the study detected a Ψ spot in addition to a U spot (lanes 4, 6, and 8). Calculation of the ratio of Ψ to U indicated that gRNA-guided U-to-Ψ conversion reached to about 2% for tRNA^{Lys}(UUT), 3% for tRNA^{Arg}(UCT), and 12% for tRNA^{Lys}(CUT) at the target site (FIG. 9C, lanes 4, 6, and 8). To check if it was possible to increase the pseudouridylation level by overexpressing a gRNA, the study created another plasmid carrying three copies of tRNA-gRNA targeting U36 of tRNA^{Lys}(CUU). Remarkably, the study detected a robust increase in pseudouridylation at position 36 (FIG. 9B, lane 9). Indeed, quantification analysis indicated that expanding the copy number of tRNA-gRNA from one to three significantly increased tRNA pseudouridylation at the target site (from 12% to close to 60%) (FIG. 9C, lane 9).

The study also quantified the pseudouridylation of the PTC (K30X) of cup1 reporter mRNA using the same quantification assay. As shown in FIG. 4D, when non-specific PTC-gRNA was expressed, only ³²pU but not ³²pΨ was detected (lane 1). However, when the PTC-specific gRNA was present, both ³²pU and ³²pΨ was detected (lane 2). Quantification indicated that gRNA-guided PTC pseudouridylation reached to ~26% (FIG. 9E, lane 2).

To exclude the possibility that the observed differences in pseudouridylation efficiency is due to different gRNA expression levels, the study also checked the gRNA level using northern analysis. The results indicated that all the gRNAs were efficiently expressed. To ensure that pseudouridylation did not alter the RNA expression, we also checked the levels of mRNA and tRNA with or without the presence of PTC-gRNA and/or tRNA-gRNA. The results showed that mRNA levels were not changed. This was expected as virtually no NMD on this PTC-containing cup1 mRNA was observed in yeast cells. Likewise, the tRNA level was essentially unchanged in response to the expression of any tRNA-gRNA. Further, the study measured the tRNA charging levels to ensure that tRNA-gRNA expression (or pseudouridylation of tRNA) did not alter tRNA charging. As expected, the results showed that uncharged tRNA and charged tRNA remained unchanged regardless of the presence or absence of a tRNA-gRNA.

The relatively low efficiency of targeted tRNA pseudouridylation is most likely due to the low accessibility caused by the stable structure of the tRNA anticodon stem-loop. To direct tRNA modification, the designer gRNAs would have to invade and disrupt the tRNA structure and base-pair with the tRNA sequence. Although the tRNA structure is relatively more difficult to target (compared to unstructured sequences), we showed that by raising the tRNA-gRNA copy number (from one to three) we drastically improved tRNA pseudouridylation efficiency (from 12% to almost 60%) (FIGS. 9B and 9C). Therefore, it is possible to address this inaccessibility issue by overexpressing tRNA-gRNA.

Experiments targeting PTC^{(UGA)} and its matching tRNA [tRNA^{Arg}(UCU)] in human cells yielded unexpected results. In fact, targeting PTC(UGA) alone [without targeting tRNA^{Arg}(UCU)] already led to solid nonsense suppression; concurrently targeting PTC^{(UGA)} and tRNA^{Arb}(UCU) did not further enhance nonsense suppression. The study hypothesized that U₃₆ of tRNA^{Arg}(UCU) is probably already naturally pseudouridylated, allowing the formation of Ψ-Ψ codon-anticodon base-pair. The suppression level probably does not depend on targeted tRNA^{Arg}(UCU) pseudouridylation because it is already modified. Rather, the level of PTC-readthrough depends on the level of targeted PTC^{(UGA)} pseudouridylation, which is likely low (refer to FIGS. 9D and 9E). Human tRNA^{Arg}(UCU) is indeed pseudouridylated. Overexpressing some near-cognate tRNAs significantly improves Ψ-mediated nonsense suppression in human cells. In particular, overexpressing tRNA^{Arg}(UCU) significantly enhances ΨGA-readthrough; however, this is not true if PTC^{(UGA)} is not pseudouridylated. Experimental results are consistent with the idea that human tRNA^{Arg}(UCU) is already naturally pseudouridylated.

### Example 8: Targeted Pseudouridylation Uncovers a Genuine T-Y Codon-Anticodon Base-Pair in Translation

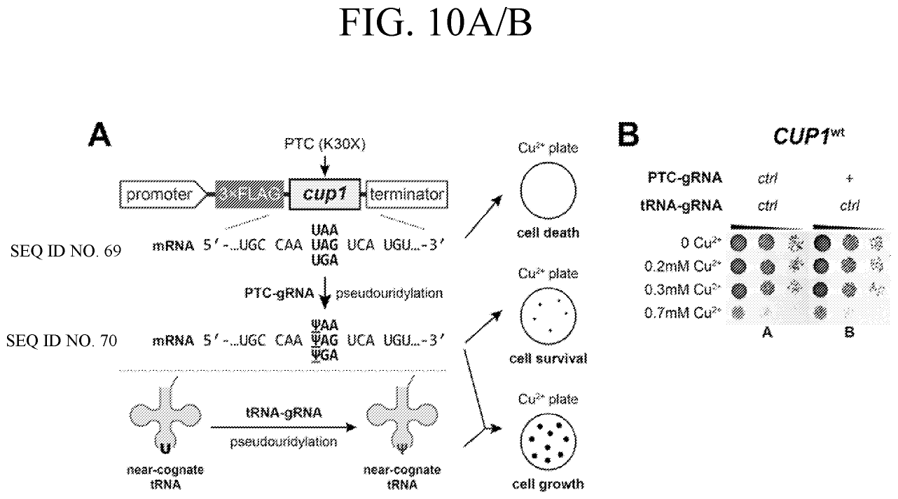

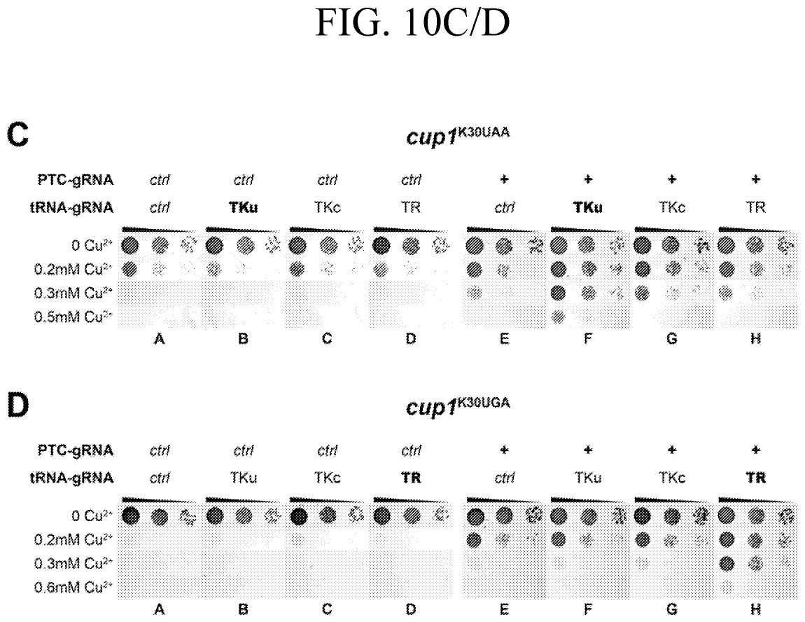

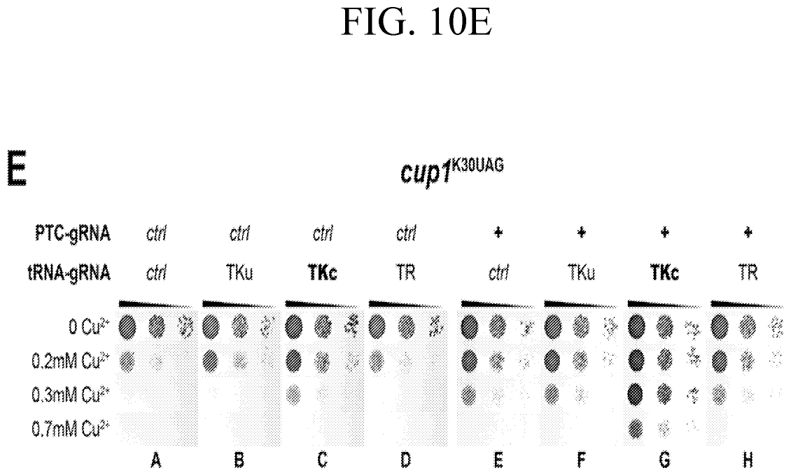

Having confirmed that PTC-gRNA and tRNA-gRNA were capable of directing pseudouridylation to the target site of target RNA, the study next tested whether targeted pseudouridylation at the PTCs or/and the anticodons of matching tRNAs would affect coding specificity, resulting in PTC-readthrough. The study took advantage of the cup1 (copper chelator) reporter system, a sensitive system permitting a quantitative analysis of PTC-readthrough through measuring cell growth in copper-containing media (FIG. 10A). Because the cup1 mRNA reporter had a PTC at codon 30 (K30X), translation of the PTC-containing mRNA generated a C-terminally truncated Cup1 protein, which was only partially functional in chelating Cu²⁺. When expressed in the cup1-deletion strain, the truncated Cup1 allowed cells to grow in media containing only low concentrations of copper (maximum 0.2 mM) (see below, FIGS. 10C, 10D, 10E, Panel A). In contrast, when the wild-type full-length Cup1 protein (with no PTC) was expressed, cells were able to grow in high copper-containing media (greater than 0.7 mM Cu²⁺) (FIG. 5B).

To analyze nonsense suppression, the study co-transformed the cup1-deletion strain (YCL46³⁸) with the PTC-containing cup1gene and a pair of gRNA genes: the PTC^{(K30X)}-gRNA (or a control PTC-gRNA with a random guide sequence) and a tRNA-gRNA. The study then assessed nonsense suppression by plating the cells on agar plates containing various concentrations of Cu²⁺.

The study first tested the K30UAA PTC (FIG. 10C). When transformed with control gRNAs, cells (cup1-deletion strain) carrying the PTC^{(K30UAA)}_cup1 could only grow in media containing up to 0.2 mM Cu²⁺, as expected (Panel A). In contrast, cells expressing a specific PTC^{(K30UAA)}-gRNA (targeting the U of UAA at codon 30) were able to grow in media containing 0.3 mM Cu²⁺ (Panel E). However, when transformed with a control PTC-gRNA and any of the three tRNA-gRNAs (targeting the tRNA anticodon), cells failed to grow in media containing higher than 0.2 mM Cu²⁺; in other words, no significant improvement was detected (Panels B-D). Even when a tRNA-gRNA specifically targeting the potential decoding tRNA [i.e., tRNA^{Lys}(UUU)] was transformed, no significant improvement was detected (Panel B). Remarkably, however, when a pair of matching gRNAs, PTC^{(K30UAA)}-gRNA and tRNA^{Lys}(UUU)-gRNA, were co-transformed, the study observed significant improvement of cell growth (from 0.3 mM Cu²⁺ to 0.5 mM Cu²⁺) (Panel F). This improvement occurred only when tRNA^{Lys}(UUU) was pseudouridylated; when the other un-matching tRNA, tRNA^{Lys}(CUU) or tRNA^{Arg}(UCU), was pseudouridylated, no improvement of cell growth was observed (cells remained to grow in media with up to 0.3 mM Cu²⁺) (Panels G and H). Because there is an intron in the pre-tRNA^{Lys}(UUU), the tRNA^{Lys}(UUU)-gRNA used above was designed to target the pre-tRNA intron sequence (FIG. 2A). To explore it further, the study designed and constructed an additional tRNA^{Lys}(UUU)-gRNA targeting the mature form of tRNA^{Lys}(UUU) (after the intron was removed). Upon transformation of both gRNAs as a mixture, the study observed a slightly less suppression when compared to the suppression by targeting pre-tRNA^{Lys}(UUU) alone, suggesting that the pre-tRNA sequence was a better target for pseudouridylation and nonsense suppression.

The study then tested the K30UGA PTC and detected a similar nonsense suppression pattern (FIG. 10D). Again, when transformed with PTC^{(K30UGA)}-cup1 and the control gRNAs (control PTC-gRNA and any of the tRNA-gRNAs), cells grew in media containing up to 0.2 mM Cu²⁺ (Panels A-D). When cells were co-transformed with the PTC^{(K30UGA})-gRNA targeting the PTC-UGA and a control tRNA-gRNA, a moderate growth improvement was detected (improved from 0.2 mM Cu²⁺ to 0.3 mM Cu²⁺) (compare Panel E with Panels A-D). Importantly, when cells were co-transformed with PTC^{(K30UGA)}-gRNA and its matching tRNA-gRNA [tRNA^{Arg}(UCU)-gRNA], cell growth was further improved (from 0.3 mM Cu²⁺ to 0.6 mM Cu²⁺) (Panel H). This improvement was specific, as co-transformation of PTC^{(K30UGA)}-gRNA and any other non-matching tRNA-gRNA [tRNA^{Lys}(UUU)-gRNA or tRNA^{Lys}(CUU)-gRNA] did not result in any improvement (Panels F and G).

Finally, the study tested the K30UAG PTC (FIG. 10E). Surprisingly, a much stronger effect on nonsense suppression was detected. First, as expected, when co-transformed with PTC^{(K30UAG)}-cup1 and PTC^{(K30UAG)}-gRNA targeting the PTC-UAG (along with a control tRNA-gRNA), cup1-deletion cells improved growth (from 0.2 mM Cu²⁺ to 0.3 mM Cu²⁺) (compare Panel E with Panel A). Second, interestingly, transformation with PTC^{(K30UAG)}-cup1 and the matching tRNA-gRNA (tRNA^{Lys}(CUU)-gRNA) targeting U36 of tRNA^{Lys}(CUU) (in the absence of a PTC-specific gRNA), also improved the cell growth from 0.2 mM Cu²⁺ to 0.3 mM Cu²⁺ (Panel C), which was not observed in other two nonsense codons (FIG. 10C, Panel B, and FIG. 10D, Panel D). It was likely due to the pseudouridylation level—pseudouridylation targeting tRNA^{Lys}(CUU) was more efficient/effective than pseudouridylation targeting tRNA^{Lys}(UUU) or tRNA^{Arg}(UCU) (FIGS. 9B and 9C, compare lane 8 with lanes 4 and 6).

Third, remarkably, when co-transformed with the matching pair of gRNAs, PTC^{(K30UAG)}-gRNA and tRNA^{Lys}(CUU)-gRNA, a robust growth improvement (close to wild-type) was observed (from 0.3 mM Cu²⁺ to greater than 0.7 mM Cu²⁺) (Panel G). Again, this was most likely due to the high level of pseudouridylation when targeting tRNA^{Lys}(CUU). This effect on nonsense suppression was also specific, since targeting any other irrelevant tRNA for pseudouridylation did not lead to improved cell growth (Panels F and H).

The nonsense suppression results presented above (from the examination of three different nonsense codons) indicated that the Ψ-Ψ base-pair contributes significantly to codon-anticodon recognition during translation.

### Example 9: The Ψ-Ψ Codon-Anticodon Pairing is Also Observed in a Completely Different Sequence Context (Different Gene)

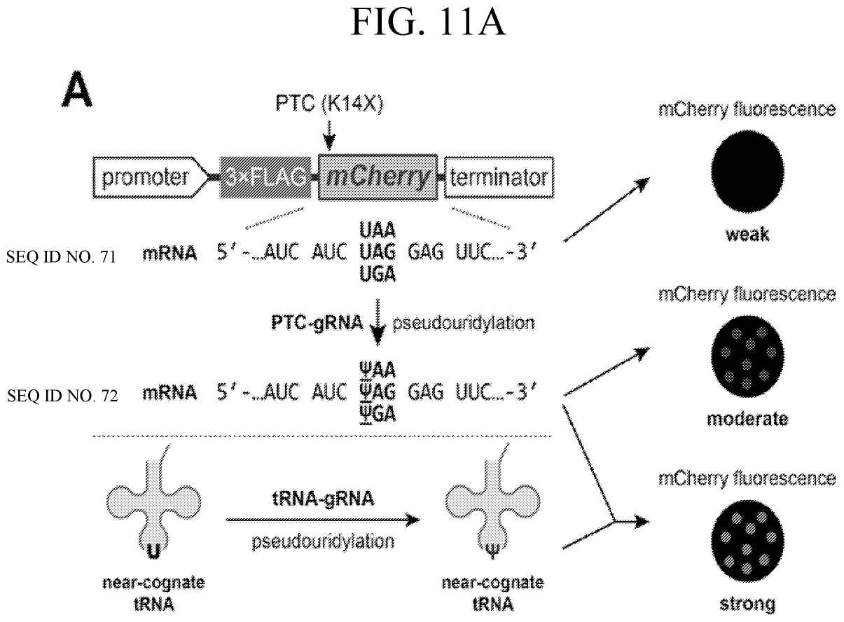

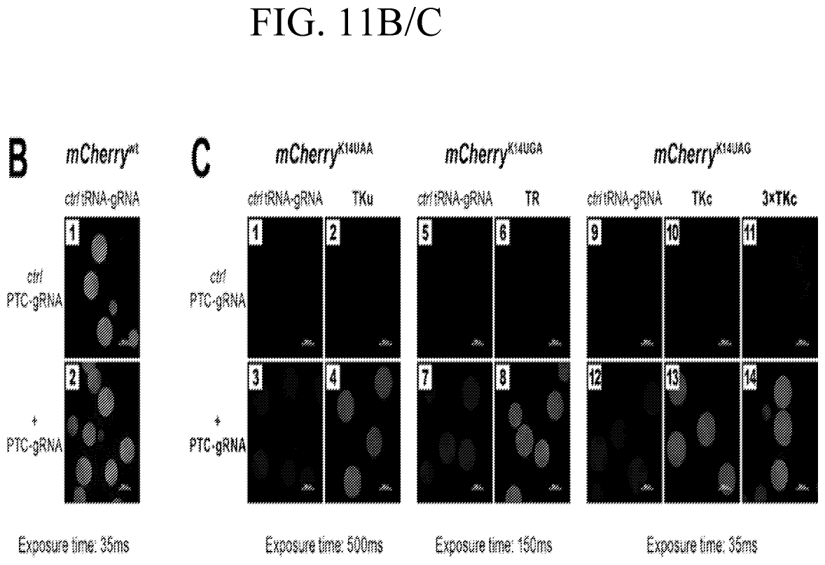

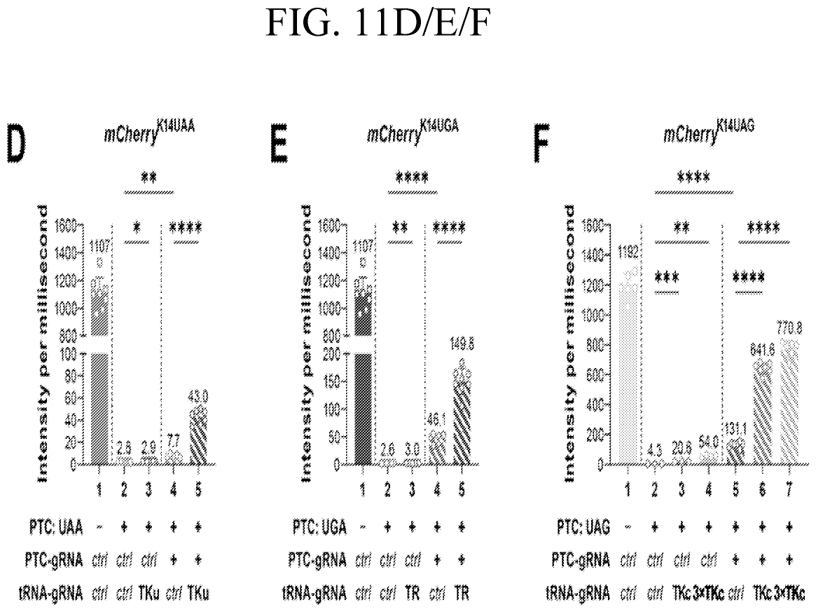

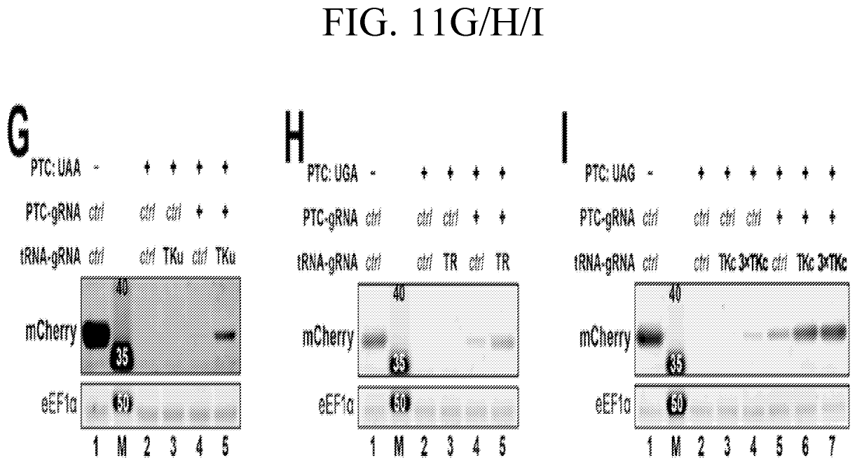

To verify this unusual Ψ-Ψ codon-anticodon pair in a different sequence context, the study created a new mCherry-based nonsense suppression reporter system, where a PTC (either TAA, TAG, or TGA) was substituted for the 14th codon (AAG) of the intron-less mCherry gene to generate mCherry^{K14UAA}, mCherry^{K14UAG}, and mCherry^{K14UGA}, respectively (FIG. 11A). Given that these mutant mCherry genes have no intron and are not NMD targets, they again allowed us to analyze only codon-anticodon base-pairing interactions during translation.

When yeast cells were transformed with the wild-type mCherry gene (no PTC), a strong mCherry fluorescence was detected (FIG. 11B); however, no mCherry fluorescence was detected when cells were transformed with any of the PTC-containing mCherry genes (FIG. 11C, Panels 1, 5, and 9), even in the presence of a control tRNA-gRNA or any of the three tRNA-gRNAs targeting tRNA^{Lys}(UUU), tRNA^{Lys}(CUU), or tRNAArg(UCU) (FIG. 11C, Panels 2, 6, and 10, and 11). However, when cells were transformed with a PTC-specific gRNA, we observed a modest (rather weak) mCherry fluorescence signal (FIG. 11C, Panels 3, 7, and 12), suggesting a readthrough full-length mCherry protein was produced, as expected. Excitingly, when cells were transformed with a matching pair of gRNAs [i.e., tRNA^{Lys}(UUU)-gRNA and PTC^{(K14UAA)}-gRNA for the PTC^{(K14UAA)}-containing mCherry, tRNALys(CUU)-gRNA and PTC^{(K14UAG)}-gRNA for the PTC^{(K14UAG)}-containing mCherry, and tRNAArg(UCU)-gRNA and PTC^{(K14UGA)}-gRNA for the PTC^{(K14UGA)}-containing mCherry], a robust mCherry fluorescence signal (5-10 fold of what was observed when a PTC-specific gRNA alone was present) was produced (FIG. 11C, Panels 4, 8, and 13). Importantly, when an mis-matching pair of gRNAs (e.g., a nonspecific tRNA-gRNA and a specific PTC-gRNA, or a specific tRNA-gRNA and a non-specific PTC-gRNA) were introduced into yeast cells, no improvement of mCherry signal was observed. Again, after quantification, we observed a much better nonsense suppression for the PTC^{(K14UAG)}-containing mCherry (54% to 65% of the wild-type level), when the gRNA pair, tRNA^{Lys}(CUU)-gRNA and PTC^{(K14UAG)}-gRNA, were co-transformed, as compared to the suppression of the other two PTCs [PTC(K14UAA)-containing mCherry and PTC^{(K14UGA)}-containing mCherry] by their respective pair of gRNAs [the tRNA^{Lys}(UUU)-gRNA and PTC^{(K14UAA)}-gRNA pair and the tRNA^{Arg}(UCU)-gRNA and PTC^{(K14UGA)}-gRNA pair](FIG. 11C, compare Panels 13 and 14 with Panel 4 and 8). Even the transformation of tRNA^{Lys}(CUU)-gRNA alone resulted in a low but clear nonsense suppression. These observed differences in nonsense suppression were again likely due to the different levels of tRNA pseudouridylation (FIGS. 9B and 9C, lanes 4, 6, 8, and 9).

The study also performed western blot experiments to examine the full-length mCherry protein production and RT-qPCR to quantitate the mCherry mRNA level. Our results showed that while the mRNA level remained virtually unchanged, the full-length protein level changed significantly (FIGS. 11G, 11H, 11I). In each case, there was an about 3-5-fold increase in cells transformed with a matching pair of gRNAs (tRNA-gRNA and PTC-gRNA) as compared to cells transformed with a PTC-specific gRNA alone or co-transformed with a PTC-specific gRNA and a mis-matching tRNA-gRNA. The western blot results were consistent with the mCherry fluorescence results presented above.

Taken together, these results indicated once again (1) that the Ψ-Ψ pair functioned as a genuine base-pair that contributed significantly to codon-anticodon recognition during translation, (2) that this Ψ-Ψ effect was specific as mis-matching pairs of gRNAs did not lead to an improved nonsense suppression, and (3) that the level of effect was proportional to the level of tRNA pseudouridylation. Pseudouridylation of tRNA^{Lys}(CUU) was much higher than that of tRNA^{Lys}(UUU) and tRNA^{Arg}(UCU) (FIGS. 9B and 9C).

### Example 10: Melting Curve Analysis Indicates that Ψ-Ψ is a High-Affinity Base Pair

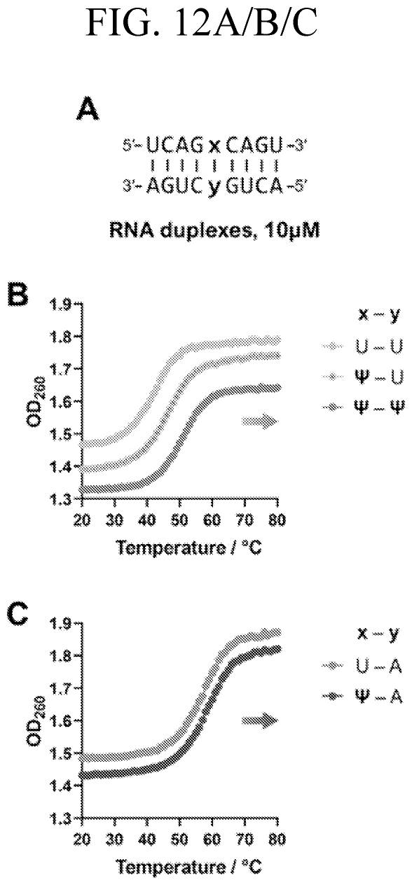

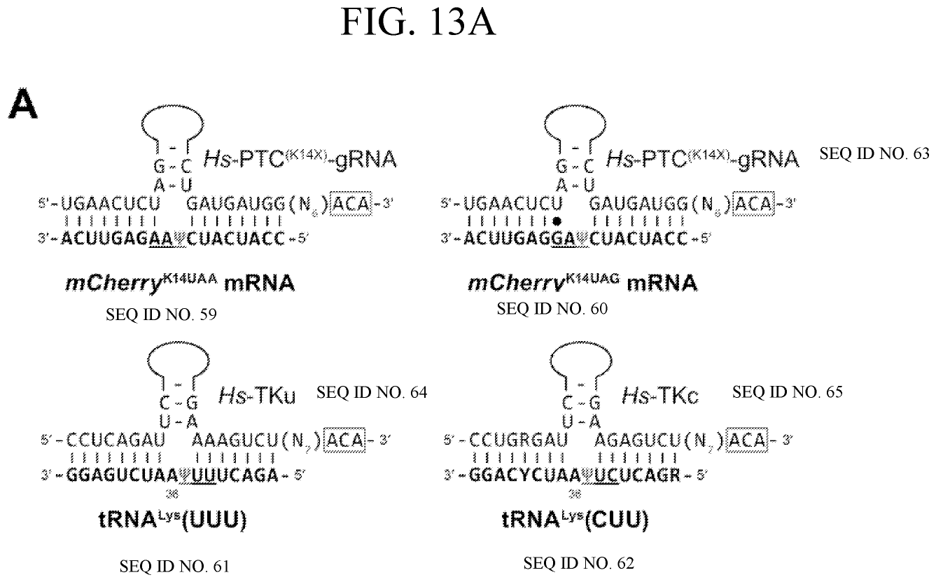

The robust improvement in nonsense suppression pointed to the high affinity of Ψ-Ψ pair. To verify this likelihood, the study carried out melting curve analysis using a well-studied double-stranded RNA oligonucleotides with a U or a Ψ in the middle of the duplex [the top strand, 5′-UCAG(U/Ψ)CAGU-3′, and the bottom complementary strand, 5′-ACUG(U/Ψ)CUGA-3′](see FIG. 12A).

As shown in FIGS. 12B and 12C, the melting curve analysis showed that the affinity of Ψ-Ψ pair, although lower than Ψ-A pair, was much higher than U-U pair and Ψ-U pair. Specifically, according to the thermodynamic calculation, the free energy value (ΔG_{37° C.}) of the above duplex was significantly lower (more negative) with Ψ-Ψ pair than with the U-U pair or Ψ-U pair. Depending on the middle base pair, either U-U, U-ΨΨ-Ψ, U-A, or Ψ-A, the melting temperatures for the duplex under 100 μM concentration were ~47° C., ~50° C., ~57° C., ~59° C., and ~61° C., respectively. The Ψ-Ψ pair duplex was much more stable than the U-U pair duplex (a ~2.51 kcal/mol difference in ΔG_{37° C.}). In comparison, there was only a ~1.05 kcal/mol difference in AG_{37° C.} between the Ψ-Ψ pair duplex and the U-A pair duplex. The study also performed the melting curving analysis to measure the affinity of Ψ-Ψ pair in a completely different sequence context and obtained consistent results.

It appeared that the high affinity of the first codon-anticodon base-pair (Ψ-Ψ rather than U-U or U-Ψ), together with the second and third Watson-Crick codon-anticodon base-pairs, would sufficiently drive PTC readthrough. These results explained, at least in part, why a Ψ-Ψ base-pair could significantly contribute to the codon-anticodon recognition during translation.

### Example 11: The Effect of Ψ-Ψ Pair on Nonsense Suppression is Also Detected in Mammalian Cells

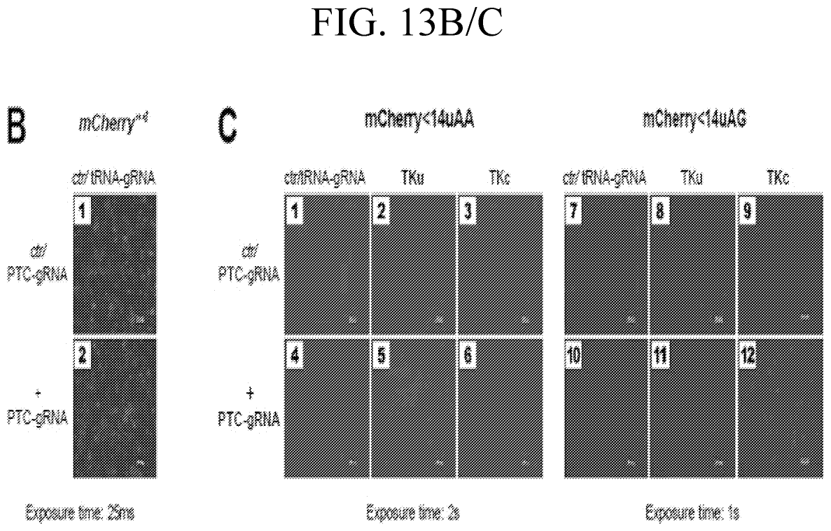

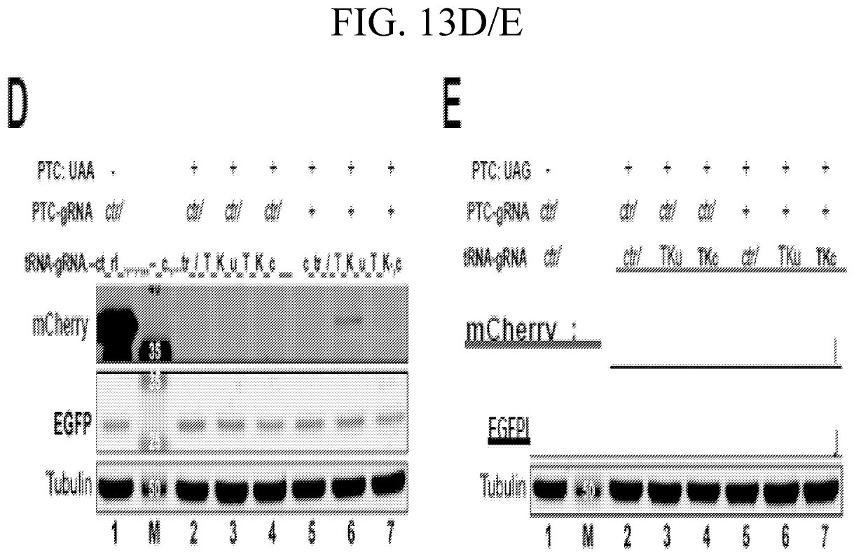

The yeast results prompted further testing whether the effect of Ψ-Ψ pairing on codon-anticodon recognition during translation could also be seen in mammalian cells. By changing the GPD promoter used in yeast to the CMV promoter for mammalian cells, the study generated a plasmid containing a mammalian cell-specific mCherry reporter gene, whose sequence, including the PTC (K14UAA, K14UAG, or K14UGA), is identical to the mCherry sequence used in the yeast experiments (FIG. 10A). The study also included an EGFP gene (for the purpose of monitoring transfection) in the plasmid. In addition, the study created gRNA plasmids as well, where a specific (for specific targeting, see FIG. 13A) or non-specific gRNA gene (or a pair of matching or non-matching gRNA genes) was included. Using this system and the same site-directed pseudouridylation strategy, the study tested the effect of Ψ-Ψ pair on nonsense suppression in HEK293 cells.

The study co-transfected HEK293 cells with the mCherry plasmid and the gRNA plasmid and carried out the same analysis as performed above (yeast experiments). Given that the similar level of GFP expression was observed for each transfection, the study inferred that transfection efficiency was similar. When the wild-type mCherry gene was transfected, very intense mCherry fluorescence signals were detected, regardless of the presence or absence of a PTC-gRNA (FIG. 13B, Panels 1 and 2). In contrast, no mCherry signals were seen when the PTC-containing mCherry gene was transfected (FIG. 13C, Panels 1 and 7). However, expressing PTC-specific gRNA alone [targeting the PTC (K14UAA or K14UAG) of mRNA] generated a low but detectable level of nonsense suppression (FIG. 13C, Panels 4 and 10). Importantly, simultaneously targeting of the PTC and the anticodon of the matching tRNA greatly enhanced nonsense suppression. Specifically, by comparing PTC^{(K14UAA)}-gRNA alone (FIG. 13C, Panel 4) with PTC^{(K14UAA)}-gRNA and tRNA^{Lys}(UUU)-gRNA together (Panel 5), the study observed a significant increase in suppression of PTC^{(K14UAA)}. For PTC^{(K14UAG)}, in the presence of PTC^{(K14UAG)}-gRNA alone, there was a low level of nonsense suppression; however, when both PTC^{(K14UAG)}-gRNA and tRNA^{Lys}(CUU)-gRNA were expressed, we detected a significant enhancement of suppression (FIG. 13C, compare Panel 10 with Panel 12). Interestingly, for PTC^{(K14UGA)}, targeting the mRNA PTC^{(K14UGA)} alone produced a high level of nonsense suppression. Simultaneously targeting the PTC and the anticodon of tRNA^{Arg}(UCU) did not further increase the suppression level. This observation was likely due to the fact that U36 of tRNA^{Arg}(UCU36) is naturally pseudouridylated

Importantly, the enhanced suppression for K14UAA and K14UAG was specific—it occurred only when the PTC and its matching tRNA were simultaneously targeted for pseudouridylation; when the PTC and its non-matching tRNA were targeted, no enhancement of nonsense suppression was observed (FIG. 13C, compare Panel 6 with Panel 4, and Panel 11 with Panel 10). Western blot was also carried out to measure the full-length read-through mCherry protein (FIGS. 13D and 13E), and the results were consistent with the mCherry fluorescence measurements discussed above (FIG. 13C).

The study also quantified the mRNA level and the levels of tRNA and tRNA charging. The results indicated that in all cases, there were no changes in the presence or absence of gRNAs, thus suggesting again that the Ψ-Ψ effect we observed was a result of codon-anticodon recognition during translation.

### Example 12: Low Level of Pseudouridylation Still Leads to High Nonsense Suppression

Despite the relative low level of targeted pseudouridylation detected on the PTC of mRNA (~26%) and the anticodon of tRNA (~2-12%), expressing the designer gRNAs that simultaneously target the PTC and its matching tRNA anticodon resulted in a significant improvement in PTC readthrough (nonsense suppression). In fact, upon targeting both the PTC and the tRNA anticodon, the study detected a ~3-10-fold increase in full-length protein level (PTC-readthrough) when compared to PTC pseudouridylation alone, reaching to ~10-65% of the wild-type level (expression of PTC-free wild-type gene).

Given the low level of pseudouridylation on both the PTC and the tRNA anticodon, the chance of the two (Ψ on two separate strands) meeting each other to form a Ψ-Ψ pair is low. Thus, the study argues that the affinity of Ψ-Ψ pair is sufficiently high with respect to codon-anticodon recognition, compensating the low level of pseudouridylation. This could explain why simultaneous pseudouridylation of both the PTC and its matching tRNA, although with low efficiency, led to a robust increase in nonsense suppression or recoding. Indeed, the direct melting curve analysis (FIG. 12) has demonstrated that the affinity/stability of the Ψ-Ψ base-pair is much higher than that of Ψ-U (or U-Ψ), which is far better than U-U pair. In other words, the affinity/stability of the Ψ-Ψ base-pair is relatively close to that of the A-U Watson-Crick base-pair. Thus, these results suggest that Ψ can not only provide flexibility in pairing with its partner (Ψ can pair with A, G, and U), but it can also provide base pairing stability.

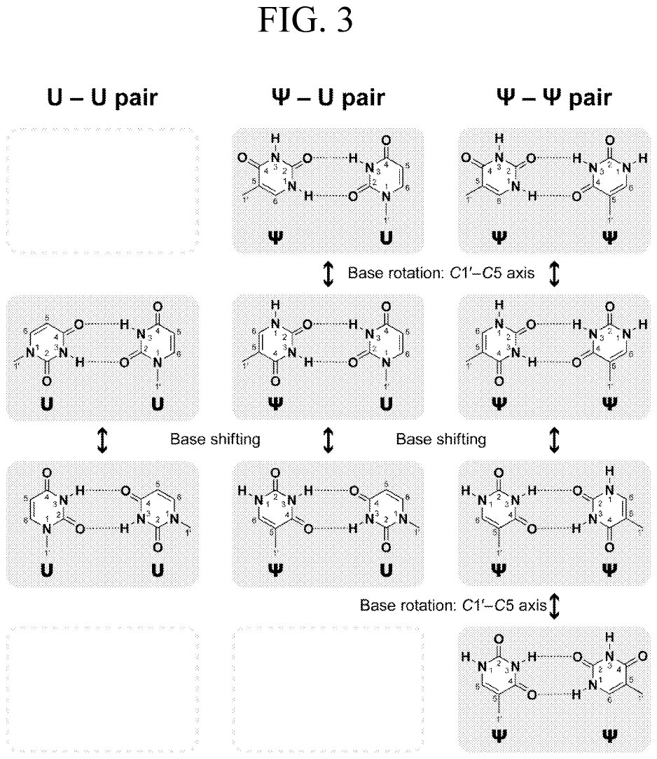

The U-U pair is not stable and rarely existent, U can form base-pairing with Ψ-U-Ψ pair. Based on the proposed U-Ψ (or Ψ-U) base pair structure (see FIG. 3, middle column), there are two hydrogen bonds between Ψ and U, generating three slightly different configurations of possible U-Ψ pair (see FIG. 3, middle column). Applying this to Ψ-Ψ pair, because there is an extra hydrogen bond donor at position 1 (—N—H) in both Ts, rotation of each base around the C1′-C5-C2 axis can, in theory, generate four different configurations of possible Ψ-Ψ base pair without altering the geometry of the backbone (see FIG. 3, right column). Thus, the probability of forming the Ψ-Ψ base pair is higher than the probability of forming the U-Ψ (or U-Ψ) pair. In addition, because it is the C—C bond in Ψ that links the base to the sugar, the rotation around the C1′-C5-C2 axis of a Ψ should be much freer as compared to the rotation around the C1′-N1-C4 axis of a U (and the N—C bond of any other known nucleotides). This makes it easier to form the four configurations of the Ψ-Ψ pair, thus arguing that the Ψ-Ψ pair is of high affinity/stability relative to the Ψ-U pair. However, the geometry of base-pairing is also a determinant in codon-anticodon recognition. Besides the affinity of the Ψ-Ψ pair, the base-pair geometry should also be considered in the context of translating ribosomes.

The results suggest that Ψ-Ψ is a genuine high-affinity base pair, pointing to the possibility that this base pair exists in nature. In this regard, this study notes that there is a Ψ₃₁-Ψ₃₉ base-pair in the anticodon stem-loop of the elongator tRNA^{Met}(CAU), and this Ψ-Ψ base-pair appears to be conserved across species from yeast to humans, suggesting that it is functionally relevant. It is likely that Ψ-Ψ base-pair also exists in other RNA species.

The experiments showed that when the PTC alone was targeted for pseudouridylation by a specific gRNA, a moderate level of nonsense suppression was detected. Targeting the anticodon of a specific tRNA alone also resulted in nonsense suppression, but the suppression level was even smaller (close to the background). This phenomenon is likely due to different pseudouridylation level at the target site Under normal circumstance where only one copy of gRNA was introduced, targeted tRNA pseudouridylation was much lower than targeted PTC pseudouridylation (2-12% versus ~26%), thus explaining why nonsense suppression by PTC pseudouridylation was more efficient than by tRNA pseudouridylation. Consistently, when three copies of tRNA-gRNA were transformed into the cell, tRNA pseudouridylation was dramatically increased. As a result, nonsense suppression was observed even when tRNA alone was targeted. Thus, gRNA-guided pseudouridylation targeting either strand (the mRNA PTC or the anticodon of the matching tRNA) could lead to nonsense suppression. However, given the low level of pseudouridylation (especially for tRNA pseudouridylation) and the low affinity of Ψ-U (or U-Ψ) base-pair (relative to the Ψ-Ψ pair), nonsense suppression generated this way was very low. With respect to specificity, PTC-pseudouridylation targets a specific PTC and is thus highly specific. In contrast, targeted tRNA pseudouridylation modifies a specific tRNA, which would probably act on all stop codons, including the PTC and the other normal stop codons. and is thus of low specificity. However, given the low level of pseudouridylation and low affinity of Ψ-U base pair, the off-target effect is likely minimum.

Only when both the PTC and the anticodon of the specific tRNA were pseudouridylated to form a Ψ-Ψ base pair did we observe a robust improvement in nonsense suppression. In a practical sense, simultaneously targeting both uridines on two matching strands would not only raise the efficiency of nonsense suppression but also increase the specificity of nonsense suppression.

It is also worth noting that most known PTCs are UAG and UGA, many of which result from Lys (AAG) and Arg (CGA), respectively. The fact that the study is able to convert UAG and UGA back to the Lys codon and Arg codon through targeted pseudouridylation suggests that the study can restore the wild-type proteins. In a clinical sense, the approach is hugely advantageous.

### Example 9: Application of Ψ-Ψ Pairing to Sense Codon-Anticodon Recognition

The targeted pseudouridylation approach has identified the novel Ψ-Ψ pair in codon-anticodon recognition during nonsense suppression. This finding raises an extremely interesting question: Is the Ψ-Ψ pair also applicable to sense codon-anticodon recognition? In other words, is it possible to target a potential U-U mis-pair in sense codon-anticodon recognition and change the near-cognate pairing (two Watson-Crick pairs and one U-U pair) to a “cognate” sense codon-anticodon pairing? If the new “cognate” tRNA proves to be able to compete with the original cognate tRNA in codon recognition, the coding/decoding specificity of a sense codon can potentially be changed. Using the same rule where the triplet pairs include both Ψ-Ψ and Watson-Crick pairs (e.g., one Ψ-Ψ pair and two conventional Watson-Crick pairs), the study has generated a list of all possible codon-anticodon pairing combinations. Specifically, from the current genetic code (64 codons), the study has identified 44 potential alternative codon-anticodon pairs and 35 of them, including 32 sense codons and three nonsense codons, would result in change in coding specificity. These 35 triplet codon-anticodon pairs, which involve Ψ-Ψ pairing, would dramatically expand the current genetic code. As a result, the expanded code would alter protein sequences (coding specificity changes for some codons) and lead to protein diversification. In this regard, the uridine of some codons and tRNA anticodons has already been known to be pseudouridylated, and it is possible that a new codon-anticodon pairing (involving a Ψ-Ψ base-pair) forms (see Table 1 below).

TABLE 1
Codon-anticodon pairs with potential U—U
mismatch. Based on the human (H. sapiens) tRNA
set (from GtRNAdb); other species may vary. U in
bold: potential U—U mismatch.

| Codon | Codon | Anticodon | Anticodon |
|---|---|---|---|
| amino acid | (4′ to 3′) | (4′ to 3′) | amino acid |
| Ala | GCU¹ | UGC² | Ala |
| Gly | GGU¹ | UCC² | Gly |
| Pro | CCU¹ | UGG² | Pro |
| Thr | ACU¹ | UGU² | Thr |
| Val | GUU¹ | UAC² | Val |
| Ser | UCU¹ | UGA² | Ser |
| Arg | CGU¹ | UCG² | Arg |
| Leu | CUU¹ | UAG² | Leu |
| Ile | AUU¹ | UAU^{2 3} | Ile |
| Phe | UUU | UAA² | Leu |
| Asn | AAU | UUU² | Lys |
| Asp | GAU | UUC² | Glu |
| His | CAU | UUG² | Gln |
| Ser | AGU | UCU² | Arg |
| Val | GUU | GUç⁴ | Asp |
| Val | GUC | GUC | Asp |
| Val | GUA | UUC | Glu |
| Val | GUG | CUC | Glu |
| Ile | AUU | GUU⁴ | Asn |
| Ile | AUC | GUU | Asn |
| Ile | AUA | UUU | Lys |
| Met | AUG | CUU | Lys |
| Leu | CUU | GUG⁴ | His |
| Leu | CUC | GUG | His |
| Leu | CUA | UUG | Gln |
| Leu | CUG | CUG | Gln |
| Phe | UUU | GUA^{4 5} | Tyr |
| Phe | UUC | GUA⁵ | Tyr |
| Cys | UGU | GCU⁴ | Ser |
| Cys | UGC | GCU | Ser |
| Stop codon | UGA | UCU^{6 7} | Arg |
| Trp | UGG | CCU | Arg |
| Tyr | UAU | GUU⁴ | Asn |
| Tyr | UAC | GUU | Asn |
| Stop codon | UAA | UUU⁶ | Lys |
| Stop codon | UAG | CUU⁶ | Lys |
| Ser | UCU | AGU⁸ | Thr |
| Ser | UCC | AGU⁸ | Thr |
| Ser | UCA | UGU | Thr |
| Ser | UCG | CGU | Thr |
| Phe | UUU | AAU⁸/GAU⁴ | Ile |
| Phe | UUC | AAU⁸/GAU | Ile |
| Leu | UUA | UAU⁹ | Ile |
| Leu | UUG | CAU | Met |

¹Amino acids unchanged (9 out of 44).
²Wobble uridine (U₃₄) involved in U—U mismatch.
³Naturally pseudouridylated at U34.
⁴Third pair: G₃₄ • U₊₃ wobble pair.
⁵Naturally pseudouridylated at U₃₅.
⁶Investigated in this work.
⁷Naturally pseudouridylated at U₃₆.
⁸Third pair: I₃₄ • U₊₃/I₃₄ • C₊₃ wobble pair.
⁹U₃₆ naturally pseudouridyalted in S. cerevisiae; unknown
in human.

Alternative coding by sense codons, manipulated by targeted pseudouridylation, also has clinical applications. It is known that a large number of inherited genetic diseases result from missense mutations. According to the proposed expanded codon-anticodon recognition code involving Ψ-Ψ pair, some of these missense mutations could be converted back to code for original amino acids after targeted pseudouridylation, thus restoring wild-type proteins. For example, assume a missense mutation from A to T occurs, converting a codon ATT (AUU in RNA coding for Isoleucine) to TTT (UUU in RNA coding for Phenylalanine). Expression of a designer box H/ACA gRNA (with two pseudouridylation pockets) simultaneously targeting the first uridine of the mutant sense codon in the mutant mRNA (converting UUU to ΨUU) and the third nucleotide (uridine) of the triplet anticodon within tRNA^{Ile}(AAU) (converting AAU to AAW) would allow this triplet codon-anticodon recognition to have one Ψ-Ψ pair and two Watson-Crick pairs (⁵′ΨUU³′-⁵′AAΨ³′). While the mutant sense codon (UUU), even if it is pseudouridylated (ΨUU), will still be recognized by its cognate tRNA, tRNA^{Phe}(GAA), the pseudouridylated tRNA^{Ile}(AAΨ) might be able to effectively compete with tRNA^{Phe}(GAA) in recognizing the pseudouridylated mutant codon (ΨUU), thus creating a protein with either Isoleucine or Phenylalanine incorporated at the mutant codon. Because Isoleucine is the original wild-type amino acid, the protein with Isoleucine being incorporated is, therefore, converted back to the wild-type protein. Given the stable Ψ-Ψ pair in codon-anticodon recognition, a substantial fraction of the protein would be converted to the wild type, which would significantly alleviate the disease phenotype.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the components and steps in any sequence which is effective to meet the objectives there intended, unless the context specifically indicates the contrary.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 17, 2025, is named “1134-259 NATL SeqList.xml” and is 122,219 bytes in size. The sequence listing contained in this AXML file is part of the specification and is hereby incorporated by reference herein in its entirety.

LIST OF SEQUENCES

| SEQ ID NO: | Description | Sequence |
|---|---|---|
| 1 | S. cerevisiae | UCCUUGUUAGCUCAGUUGGUAGAGCGUUCGGCUUUUAACCGAA |
| | tRNA^{Lys}(UUU) | AUGUCAGGGGUUCGAGCCCCCUAUGAGGAG |
| 2 | S. cerevisiae | GCCUUGUUGGCGCAAUCGGUAGCGCGUAUGACUCUUAAUCAUA |
| | tRNA^{Lys}(CUU) | AGGUUAGGGGUUCGAGCCCCCUACAGGGCU |
| 3 | S. cerevisiae S | GCUCGCGUGGCGUAAUGGCAACGCGUCUGACUUCUAAUCAGAA |
| | tRNA^{Arg}(UCU) | GAUUAUGGGUUCGACCCCCAUCGUGAGUG |
| 4 | Human | GCCCGGAUAGCUCAGUCGGUAGAGCAUCAGACUUUUAAUCUGA |
| | tRNA^{Lys}(UUU) | GGGUCCAGGGUUCAAGUCCCUGUUCGGGCG |
| 5 | Human | GCCCGGCUAGCUCAGUCGGUAGAGCAUGAGACUCUUAAUCUCA |
| | tRNA^{Lys}(CUU) | GGGUCGUGGGUUCGAGCCCCACGUUGGGCG |
| 6 | Human | GGCUCUGUGGCGCAAUGGAUAGCGCAUUGGACUUCUAAUUCAA |
| | tRNA^{Arg}(UCU) | AGGUUGUGGGUUCGAGUCCCACCAGAGUCG |
| 7 | Targeting molecule | GGGACUGAAUGCGCUCCGGCGAGGCAGCCCACAUCAAGUGGAAC |
| | for S cerevisiae: | UACACAGACUUCCUUGUCGCAAAGCCGCGGUCCCAAGAGCAAUC |
| | tRNA^{Lys}(UUU) | CUUCCCGAUGCGCUGCUCAUCUAUUAAAAUCAAUAGUAGAAUG |
| | | UAGUAAAGCCGACGGGAGACAUUC |
| 8 | Targeting molecule | GGGACUGCUUAUGAUCCGGCGAGGCAGCCCACAUCAAGUGGAA |
| | for S cerevisiae: | CUACACAGACUUCCUUGUCGCAGAGUCACGGUCCCAAGAGCAAU |
| | tRNA^{Lys}(CUU) | CCUUCCCGUUAUGAUGCUCAUCUAUUAAAAUCAAUAGUAGAAU |
| | | GUAGUAGAGUCAUCGGGAGACAUUC |
| 9 | Targeting molecule | GGGACCUCUUCUGAUACGGCGAGCCUGCCCACAUCAAGUGGAAC |
| | for S cerevisiae: | UACACAGACAUGCUUGUCGCGAAGUCACGGUCCCAAGAGCAAUC |
| | tRNA^{Arg}(UCU) | CUUCCCGUUCUGAUGCUCAUCUAUUAAAAUCAAUAGUAGAAUG |
| | | UAGUGAAGUCAGCGGGAGACAUUC |
| 10 | Targeting molecule | CCACACACCUCAGAUCCAGUCCAGGGCAGCUUCCCUGUUCUGCA |
| | for human | AAGUCUUGUGUGGACAUUAAAAUUGCGACCUCAGAUCCAGUAU |
| | tRNA^{Lys}(UUU) | AACUAUAAAUAGUAAUGCUGCAAAGUCUUCGCAGACAAAA |
| 11 | Targeting molecule | CCACACACCUGAGAUCCAGUCCAGGGCAGCUUCCCUGUUCUGCA |
| | for human | GAGUCUUGUGUGGACAUUAAAAUUGCGACCUGAGAUCCAGUAU |
| | tRNA^{Lys}(CUU) | AACUAUAAAUAGUAAUGCUGCAGAGUCUUCGCAGACAAAA |
| 12 | Targeting molecule | CCACACACUUUGGAUACAGUCCAGGGCAGCUUCCCUGUUCUGCG |
| | for human | AAGUCCUGUGUGGACAUUAAAAUUGCCUCUUUGGAUACAGUAU |
| | tRNA^{Arg}(UCU) | AACUAUAAAUAGUAAUGCUGCGAAGUCCAGGCAGACAAAA |
| 13 | Modified | UCCUUGUUAGCUCAGUUGGUAGAGCGUUCGGCUUUΨAACCGAA |
| | S. cerevisiae | AUGUCAGGGGUUCGAGCCCCCUAUGAGGAG |
| | tRNA^{Lys}(UUU) | |
| 14 | Modified | Modified tRNA^{Lys}(CUU)- |
| | S. cerevisiae | GCCUUGUUGGCGCAAUCGGUAGCGCGUAUGACUCUΨAAUCAUA |
| | tRNA^{Lys}(CUU) | AGGUUAGGGGUUCGAGCCCCCUACAGGGCU |
| 15 | Modified | GCUCGCGUGGCGUAAUGGCAACGCGUCUGACUUCΨAAUCAGAA |
| | S. cerevisiae S | GAUUAUGGGUUCGACCCCCAUCGUGAGUG |
| | tRNA^{Arg}(UCU) | |
| 16 | Modified human | GCCCGGAUAGCUCAGUCGGUAGAGCAUCAGACUUUYAAUCUGA |
| | tRNA^{Lys}(UUU) | GGGUCCAGGGUUCAAGUCCCUGUUCGGGCG |
| 17 | Modified human | GCCCGGCUAGCUCAGUCGGUAGAGCAUGAGACUCUTAAUCUCA |
| | tRNA^{Lys}(CUU) | GGGUCGUGGGUUCGAGCCCCACGUUGGGCG |
| 18 | Modified human | GGCUCUGUGGCGCAAUGGAUAGCGCAUUGGACUUCAAUUCAA |
| | tRNA^{Arg}(UCU) | AGGUUGUGGGUUCGAGUCCCACCAGAGUCG |
| 19 | mRNA targeting | GGCACAACCUUCUGACCUGCUUUCUCUUAUGUGAGUAGUGUUA |
| | nucleic acid | AGAACUAUGUGCUAUACAAAUAAUUGCAGCACCUUCUGCAGUA |
| | molecule for | UAACUAUAAAUAGUAAUGCUGCAAGAACUCUGCAGACAAAA |
| | pseudouridylation | |
| 20 | Plasmid vector | TCTTCCGCTTCCTCGCTCACTGACTCGCTGCGCTCGGTCGTTCGGC |
| | sequence for | TGCGGCGAGCGGTATCAGCTCACTCAAAGGCGGTAATACGGTTAT |
| | S. cerevisiae | CCACAGAATCAGGGGATAACGCAGGAAAGAACATGTGAGCAAAA |
| | mRNA-tRNA | GGCCAGCAAAAGGCCAGGAACCGTAAAAAGGCCGCGTTGCTGGC |
| | | GTTTTTCCATAGGCTCCGCCCCCCTGACGAGCATCACAAAAATCG |
| | | ACGCTCAAGTCAGAGGTGGCGAAACCCGACAGGACTATAAAGAT |
| | | ACCAGGCGTTTCCCCCTGGAAGCTCCCTCGTGCGCTCTCCTGTTCC |
| | | GACCCTGCCGCTTACCGGATACCTGTCCGCCTTTCTCCCTTCGGGA |
| | | AGCGTGGCGCTTTCTCATAGCTCACGCTGTAGGTATCTCAGTTCGG |
| | | TGTAGGTCGTTCGCTCCAAGCTGGGCTGTGTGCACGAACCCCCCG |
| | | TTCAGCCCGACCGCTGCGCCTTATCCGGTAACTATCGTCTTGAGTC |
| | | CAACCCGGTAAGACACGACTTATCGCCACTGGCAGCAGCCACTGG |
| | | TAACAGGATTAGCAGAGCGAGGTATGTAGGCGGTGCTACAGAGTT |
| | | CTTGAAGTGGTGGCCTAACTACGGCTACACTAGAAGAACAGTATT |
| | | TGGTATCTGCGCTCTGCTGAAGCCAGTTACCTTCGGAAAAAGAGT |
| | | TGGTAGCTCTTGATCCGGCAAACAAACCACCGCTGGTAGCGGTGG |
| | | TTTTTTTGTTTGCAAGCAGCAGATTACGCGCAGAAAAAAAGGATC |
| | | TCAAGAAGATCCTTTGATCTTTTCTACGGGGTCTGACGCTCAGTGG |
| | | AACGAAAACTCACGTTAAGGGATTTTGGTCATGAGATTATCAAAA |
| | | AGGATCTTCACCTAGATCCTTTTAAATTAAAAATGAAGTTTTAAAT |
| | | CAATCTAAAGTATATATGAGTAAACTTGGTCTGACAGTTACCAAT |
| | | GCTTAATCAGTGAGGCACCTATCTCAGCGATCTGTCTATTTCGTTC |
| | | ATCCATAGTTGCCTGACTCCCCGTCGTGTAGATAACTACGATACG |
| | | GGAGGGCTTACCATCTGGCCCCAGTGCTGCAATGATACCGCGAGA |
| | | CCCACGCTCACCGGCTCCAGATTTATCAGCAATAAACCAGCCAGC |
| | | CGGAAGGGCCGAGCGCAGAAGTGGTCCTGCAACTTTATCCGCCTC |
| | | CATCCAGTCTATTAATTGTTGCCGGGAAGCTAGAGTAAGTAGTTC |
| | | GCCAGTTAATAGTTTGCGCAACGTTGTTGCCATTGCTACAGGCATC |
| | | GTGGTGTCACGCTCGTCGTTTGGTATGGCTTCATTCAGCTCCGGTT |
| | | CCCAACGATCAAGGCGAGTTACATGATCCCCCATGTTGTGCAAAA |
| | | AAGCGGTTAGCTCCTTCGGTCCTCCGATCGTTGTCAGAAGTAAGTT |
| | | GGCCGCAGTGTTATCACTCATGGTTATGGCAGCACTGCATAATTCT |
| | | CTTACTGTCATGCCATCCGTAAGATGCTTTTCTGTGACTGGTGAGT |
| | | ACTCAACCAAGTCATTCTGAGAATAGTGTATGCGGCGACCGAGTT |
| | | GCTCTTGCCCGGCGTCAATACGGGATAATACCGCGCCACATAGCA |
| | | GAACTTTAAAAGTGCTCATCATTGGAAAACGTTCTTCGGGGCGAA |
| | | AACTCTCAAGGATCTTACCGCTGTTGAGATCCAGTTCGATGTAAC |
| | | CCACTCGTGCACCCAACTGATCTTCAGCATCTTTTACTTTCACCAG |
| | | CGTTTCTGGGTGAGCAAAAACAGGAAGGCAAAATGCCGCAAAAA |
| | | AGGGAATAAGGGCGACACGGAAATGTTGAATACTCATACTCTTCC |
| | | TTTTTCAATATTATTGAAGCATTTATCAGGGTTATTGTCTCATGAG |
| | | CGGATACATATTTGAATGTATTTAGAAAAATAAACAAATAGGGGT |
| | | TCCGCGCACATTTCCCCGAAAAGTGCCACCTGACGTCTAAGAAAC |
| | | CATTATTATCATGACATTAACCTATAAAAATAGGCGTATCACGAG |
| | | GCCCTTTCGTCTTCAAGAATTAACTGTGGGAATACTCAGGTATCGT |
| | | AAGATGCAAGAGTTCGAATCTCTTAGCAACCATTATTTTTTTCCTC |
| | | AACATAACGAGAACACACAGGGGCGCTATCGCACAGAATCAAAT |
| | | TCGATGACTGGAAATTTTTTGTTAATTTCAGAGGTCGCCTGACGCA |
| | | TATACCTTTTTCAACTGAAAAATTGGGAGAAAAAGGAAAGGTGAG |
| | | AGCCGCGGAACCGGCTTTTCATATAGAATAGAGAAGCGTTCATGA |
| | | CTAAATGCTTGCATCACAATACTTGAAGTTGACAATATTATTTAAG |
| | | GACCTATTGTTTTTTCCAATAGGTGGTTAGCAATCGTCTTACTTTC |
| | | TAACTTTTCTTACCTTTTACATTTCAGCAATATATATATATATATTT |
| | | CAAGGATATACCATTCTAATGTCTGCCCCTAAGAAGATCGTCGTTT |
| | | TGCCAGGTGACCACGTTGGTCAAGAAATCACAGCCGAAGCCATTA |
| | | AGGTTCTTAAAGCTATTTCTGATGTTCGTTCCAATGTCAAGTTCGA |
| | | TTTCGAAAATCATTTAATTGGTGGTGCTGCTATCGATGCTACAGGT |
| | | GTCCCACTTCCAGATGAGGCGCTGGAAGCCTCCAAGAAGGTTGAT |
| | | GCCGTTTTGTTAGGTGCTGTGGGTGGTCCTAAATGGGGTACAGGT |
| | | AGTGTTAGACCTGAACAAGGTTTACTAAAAATCCGTAAAGAACTT |
| | | CAATTGTACGCCAACTTAAGACCATGTAACTTTGCATCCGACTCTC |
| | | TTTTAGACTTATCTCCAATCAAGCCACAATTTGCTAAAGGTACTGA |
| | | CTTCGTTGTTGTCAGAGAATTAGTGGGAGGTATTTACTTTGGTAAG |
| | | AGAAAGGAAGACGATGGTGATGGTGTCGCTTGGGATAGTGAACA |
| | | ATACACCGTTCCAGAAGTGCAAAGAATCACAAGAATGGCCGCTTT |
| | | CATGGCCCTACAACATGAGCCACCATTGCCTATTTGGTCCTTGGAT |
| | | AAAGCTAATGTTTTGGCCTCTTCAAGATTATGGAGAAAAACTGTG |
| | | GAGGAAACCATCAAGAACGAATTTCCTACATTGAAGGTTCAACAT |
| | | CAATTGATTGATTCTGCCGCCATGATCCTAGTTAAGAACCCAACC |
| | | CACCTAAATGGTATTATAATCACCAGCAACATGTTTGGTGATATC |
| | | ATCTCCGATGAAGCCTCCGTTATCCCAGGTTCCTTGGGTTTGTTGC |
| | | CATCTGCGTCCTTGGCCTCTTTGCCAGACAAGAACACCGCATTTGG |
| | | TTTGTACGAACCATGCCACGGTTCTGCTCCAGATTTGCCAAAGAA |
| | | TAAGGTCAACCCTATCGCCACTATCTTGTCTGCTGCAATGATGTTG |
| | | AAATTGTCATTGAACTTGCCTGAAGAAGGTAAGGCCATTGAAGAT |
| | | GCAGTTAAAAAGGTTTTGGATGCAGGTATCAGAACTGGTGATTTA |
| | | GGTGGTTCCAACAGTACCACGGAAGTCGGTGATGCTGTCGCCGAA |
| | | GAAGTTAAGAAAATCCTTGCTTAAAAAGATTCTCTTTTTTTATGAT |
| | | ATTTGTACATAAACTTTATAAATGAAATTCATAATAGAAACGACA |
| | | CGAAATTACAAAATGGAATATGTTCATAGGGTAGAATTAATTCTC |
| | | ATGTTTGACAGCTTATCATCGGATCGATCCAATATCAAAGGAAAT |
| | | GATAGCATTGAAGGATGAGACTAATCCAATTGAGGAGTGGCAGC |
| | | ATATAGAACAGCTAAAGGGTAGTGCTGAAGGAAGCATACGATAC |
| | | CCCGCATGGAATGGGATAATATCACAGGAGGTACTAGACTACCTT |
| | | TCATCCTACATAAATAGACGCATATAAGTACGCATTTAAGCATAA |
| | | ACACGCACTATGCCGTTCTTCTCATGTATATATATATACAGGCAAC |
| | | ACGCAGATATAGGTGCGACGTGAACAGTGAGCTGTATGTGCGCAG |
| | | CTCGCGTTGCATTTTCGGAAGCGCTCGTTTTCGGAAACGCTTTGAA |
| | | GTTCCTATTCCGAAGTTCCTATTCTCTAGCTAGAAAGTATAGGAAC |
| | | TTCAGAGCGCTTTTGAAAACCAAAAGCGCTCTGAAGACGCACTTT |
| | | CAAAAAACCAAAAACGCACCGGACTGTAACGAGCTACTAAAATA |
| | | TTGCGAATACCGCTTCCACAAACATTGCTCAAAAGTATCTCTTTGC |
| | | TATATATCTCTGTGCTATATCCCTATATAACCTACCCATCCACCTT |
| | | TCGCTCCTTGAACTTGCATCTAAACTCGACCTCTACATTTTTTATG |
| | | TTTATCTCTAGTATTACTCTTTAGACAAAAAAATTGTAGTAAGAAC |
| | | TATTCATAGAGTGAATCGAAAACAATACGAAAATGTAAACATTTC |
| | | CTATACGTAGTATATAGAGACAAAATAGAAGAAACCGTTCATAAT |
| | | TTTCTGACCAATGAAGAATCATCAACGCTATCACTTTCTGTTCACA |
| | | AAGTATGCGCAATCCACATCGGTATAGAATATAATCGGGGATGCC |
| | | TTTATCTTGAAAAAATGCACCCGCAGCTTCGCTAGTAATCAGTAA |
| | | ACGCGGGAAGTGGAGTCAGGCTTTTTTTATGGAAGAGAAAATAGA |
| | | CACCAAAGTAGCCTTCTTCTAACCTTAACGGACCTACAGTGCAAA |
| | | AAGTTATCAAGAGACTGCATTATAGAGCGCACAAAGGAGAAAAA |
| | | AAGTAATCTAAGATGCTTTGTTAGAAAAATAGCGCTCTCGGGATG |
| | | CATTTTTGTAGAACAAAAAAGAAGTATAGATTCTTTGTTGGTAAA |
| | | ATAGCGCTCTCGCGTTGCATTTCTGTTCTGTAAAAATGCAGCTCAG |
| | | ATTCTTTGTTTGAAAAATTAGCGCTCTCGCGTTGCATTTTTGTTTTA |
| | | CAAAAATGAAGCACAGATTCTTCGTTGGTAAAATAGCGCTTTCGC |
| | | GTTGCATTTCTGTTCTGTAAAAATGCAGCTCAGATTCTTTGTTTGA |
| | | AAAATTAGCGCTCTCGCGTTGCATTTTTGTTCTACAAAATGAAGCA |
| | | CAGATGCTTCGTTAACAAAGATATGCTATTGAAGTGCAAGATGGA |
| | | AACGCAGAAAATGAACCGGGGATGCGACGTGCAAGATTACCTAT |
| | | GCAATAGATGCAATAGTTTCTCCAGGAACCGAAATACATACATTG |
| | | TCTTCCGTAAAGCGCTAGACTATATATTATTATACAGGTTCAAATA |
| | | TACTATCTGTTTCAGGGAAAACTCCCAGGTTCGGATGTTCAAAATT |
| | | CAATGATGGGTAACAAGTACGATCCGATATATGCGGTGTGAAATA |
| | | CCGCACAGATGCGTAAGGAGAAAATACCGCATCAGGCGCCATTC |
| | | GCCATTCAGGCTGCGCAACTGTTGGGAAGGGCGATCGGTGCGGGC |
| | | CTCTTCGCTATTACGCCAGCTGGCGAAAGGGGGATGTGCTGCAAG |
| | | GCGATTAAGTTGGGTAACGCCAGGGTTTTCCCAGTCACGACGTTG |
| | | TAAAACGACGGCCAGTGAATTCGTCGAGTTTATCATTATCAATAC |
| | | TGCCATTTCAAAGAATACGTAAATAATTAATAGTAGTGATTTTCCT |
| | | AACTTTATTTAGTCAAAAAATTAGCCTTTTAATTCTGCTGTAACCC |
| | | GTACATGCCCAAAATAGGGGGGGGTTACACAGAATATATAACAT |
| | | CGTAGGTGTCTGGGTGAACAGTTTATTCCTGGCATCCACTAAATAT |
| | | AATGGAGCCCGCTTTTTAAGCTGGCATCCAGAAAAAAAAAGAATC |
| | | CCAGCACCAAAATATTGTTTTCTTCACCAACCATCAGTTCATAGGT |
| | | CCATTCTCTTAGCGCAACTACAGAGAACAGGGGCACAAACAGGC |
| | | AAAAAACGGGCACAACCTCAATGGAGTGATGCAACCTGCCTGGA |
| | | GTAAATGATGACACAAGGCAATTGACCCACGCATGTATCTATCTC |
| | | ATTTTCTTACACCTTCTATTACCTTCTGCTCTCTCTGATTTGGAAAA |
| | | AGCTGAAAAAAAAGGTTGAAACCAGTTCCCTGAAATTATTCCCCT |
| | | ACTTGACTAATAAGTATATAAAGACGGTAGGTATTGATTGTAATT |
| | | CTGTAAATCTATTTCTTAAACTTCTTAAATTCTACTTTTATAGTTAG |
| | | TCTTTTTTTTAGTTTTAAAACACCAAGAACTTAGTTTCGACGGATC |
| | | CTTTTTATTTCTTTCTAAGTGGGTACTGGCAGGAGTCGGGGCCTAG |
| | | TTTAGAGAGAAGTAGACTCAGTCGACAAGAGCTCTTTTTATTTCTT |
| | | TCTAAGTGGGTACTGGCAGGAGTCGGGGCCTAGTTTAGAGAGAAG |
| | | TAGACTCAGGTACCAACTCGAGTTTTTATTTCTTTCTAAGTGGGTA |
| | | CTGGCAGGAGTCGGGGCCTAGTTTAGAGAGAAGTAGACTCAGCTA |
| | | GCAACTGCAGAGTAATCCTTCTTACATTGTATCGTAGCGCTGCATA |
| | | TATAATGCGTAAAATTTTCAAGCTTGGCGTAATCATGGTCATAGCT |
| | | GTTTCCTGTGTGAAATTGTTATCCGCTCACAATTCCACACAACATA |
| | | CGAGCCGGAAGCATAAAGTGTAAAGCCTGGGGTGCCTAATGAGT |
| | | GAGCTAACTCACATTAATTGCGTTGCGCTCACTGCCCGCTTTCCAG |
| | | TCGGGAAACCTGTCGTGCCAGCTGCATTAATGAATCGGCCAACGC |
| | | GCGGGGAGAGGCGGTTTGCGTATTGGGCGC |
| 21 | Plasmid vector | TAGTTATTAATAGTAATCAATTACGGGGTCATTAGTTCATAGCCCA |
| | sequence for human | TATATGGAGTTCCGCGTTACATAACTTACGGTAAATGGCCCGCCT |
| | mRNA-tRNA | GGCTGACCGCCCAACGACCCCCGCCCATTGACGTCAATAATGACG |
| | construct | TATGTTCCCATAGTAACGCCAATAGGGACTTTCCATTGACGTCAAT |
| | | GGGTGGAGTATTTACGGTAAACTGCCCACTTGGCAGTACATCAAG |
| | | TGTATCATATGCCAAGTACGCCCCCTATTGACGTCAATGACGGTA |
| | | AATGGCCCGCCTGGCATTATGCCCAGTACATGACCTTATGGGACT |
| | | TTCCTACTTGGCAGTACATCTACGTATTAGTCATCGCTATTACCAT |
| | | GGTGATGCGGTTTTGGCAGTACATCAATGGGCGTGGATAGCGGTT |
| | | TGACTCACGGGGATTTCCAAGTCTCCACCCCATTGACGTCAATGG |
| | | GAGTTTGTTTTGGCACCAAAATCAACGGGACTTTCCAAAATGTCG |
| | | TAACAACTCCGCCCCATTGACGCAAATGGGCGGTAGGCGTGTACG |
| | | GTGGGAGGTCTATATAAGCAGAGCTGGTTTAGTGAACCGTCAGAT |
| | | CCAATACTATTCCACTTCTACTCAAATCCGCCACCATGGTGAGCA |
| | | AGGGCGAGGAGCTGTTCACCGGGGTGGTGCCCATCCTGGTCGAGC |
| | | TGGACGGCGACGTAAACGGCCACAAGTTCAGCGTGTCCGGCGAG |
| | | GGCGAGGGCGATGCCACCTACGGCAAGCTGACCCTGAAGTTCATC |
| | | TGCACCACCGGCAAGCTGCCCGTGCCCTGGCCCACCCTCGTGACC |
| | | ACCCTGACCTACGGCGTGCAGTGCTTCAGCCGCTACCCCGACCAC |
| | | ATGAAGCAGCACGACTTCTTCAAGTCCGCCATGCCCGAAGGCTAC |
| | | GTCCAGGAGCGCACCATCTTCTTCAAGGACGACGGCAACTACAAG |
| | | ACCCGCGCCGAGGTGAAGTTCGAGGGCGACACCCTGGTGAACCG |
| | | CATCGAGCTGAAGGGCATCGACTTCAAGGAGGACGGCAACATCCT |
| | | GGGGCACAAGCTGGAGTACAACTACAACAGCCACAACGTCTATAT |
| | | CATGGCCGACAAGCAGAAGAACGGCATCAAGGTGAACTTCAAGA |
| | | TCCGCCACAACATCGAGGACGGCAGCGTGCAGCTCGCCGACCACT |
| | | ACCAGCAGAACACCCCCATCGGCGACGGCCCCGTGCTGCTGCCCG |
| | | ACAACCACTACCTGAGCACCCAGTCCGCCCTGAGCAAAGACCCCA |
| | | ACGAGAAGCGCGATCACATGGTCCTGCTGGAGTTCGTGACCGCCG |
| | | CCGGGATCACTCTCGGCATGGACGAGCTGTACAAGTAAAGCGGCC |
| | | GCGACTCTAGATCATAATCAGCCATACCACATTTGTAGAGGTTTT |
| | | ACTTGCTTTAAAAAACCTCCCACACCTCCCCCTGAACCTGAAACA |
| | | TAAAATGAATGCAATTGTTGTTGTTAACTTGTTTATTGCAGCTTAT |
| | | AATGGTTACAAATAAAGCAATAGCATCACAAATTTCACAAATAAA |
| | | GCATTTTTTTCACTGCATTCTAGTTGTGGTTTGTCCAAACTCATCA |
| | | ATGTATCTTAAGGCGTAAATTGTAAGCGTTAATATTTTGTTAAAAT |
| | | TCGCGTTAAATTTTTGTTAAATCAGCTCATTTTTTAACCAATAGGC |
| | | CGAAATCGGCAAAATCCCTTATAAATCAAAAGAATAGACCGAGA |
| | | TAGGGTTGAGTGTTGTTCCAGTTTGGAACAAGAGTCCACTATTAA |
| | | AGAACGTGGACTCCAACGTCAAAGGGCGAAAAACCGTCTATCAG |
| | | GGCGATGGCCCACTACGTGAACCATCACCCTAATCAAGTTTTTTG |
| | | GGGTCGAGGTGCCGTAAAGCACTAAATCGGAACCCTAAAGGGAG |
| | | CCCCCGATTTAGAGCTTGACGGGGAAAGCCGGCGAACGTGGCGA |
| | | GAAAGGAAGGGAAGAAAGCGAAAGGAGCGGGCGCTAGGGCGCT |
| | | GGCAAGTGTAGCGGTCACGCTGCGCGTAACCACCACACCCGCCGC |
| | | GCTTAATGCGCCGCTACAGGGCGCGTCAGGTGGCACTTTTCGGGG |
| | | AAATGTGCGCGGAACCCCTATTTGTTTATTTTTCTAAATACATTCA |
| | | AATATGTATCCGCTCATGAGACAATAACCCTGATAAATGCTTCAA |
| | | TAATATTGAAAAAGGAAGAGTCCTGAGGCGGAAAGAACCAGCTG |
| | | TGGAATGTGTGTCAGTTAGGGTGTGGAAAGTCCCCAGGCTCCCCA |
| | | GCAGGCAGAAGTATGCAAAGCATGCATCTCAATTAGTCAGCAACC |
| | | AGGTGTGGAAAGTCCCCAGGCTCCCCAGCAGGCAGAAGTATGCA |
| | | AAGCATGCATCTCAATTAGTCAGCAACCATAGTCCCGCCCCTAAC |
| | | TCCGCCCATCCCGCCCCTAACTCCGCCCAGTTCCGCCCATTCTCCG |
| | | CCCCATGGCTGACTAATTTTTTTTATTTATGCAGAGGCCGAGGCCG |
| | | CCTCGGCCTCTGAGCTATTCCAGAAGTAGTGAGGAGGCTTTTTTG |
| | | GAGGCCTAGGCTTTTGCAAAGATCGATCAAGAGACAGGATGAGG |
| | | ATCGTTTCGCATGATTGAACAAGATGGATTGCACGCAGGTTCTCC |
| | | GGCCGCTTGGGTGGAGAGGCTATTCGGCTATGACTGGGCACAACA |
| | | GACAATCGGCTGCTCTGATGCCGCCGTGTTCCGGCTGTCAGCGCA |
| | | GGGGCGCCCGGTTCTTTTTGTCAAGACCGACCTGTCCGGTGCCCTG |
| | | AATGAACTGCAAGACGAGGCAGCGCGGCTATCGTGGCTGGCCAC |
| | | GACGGGCGTTCCTTGCGCAGCTGTGCTCGACGTTGTCACTGAAGC |
| | | GGGAAGGGACTGGCTGCTATTGGGCGAAGTGCCGGGGCAGGATC |
| | | TCCTGTCATCTCACCTTGCTCCTGCCGAGAAAGTATCCATCATGGC |
| | | TGATGCAATGCGGCGGCTGCATACGCTTGATCCGGCTACCTGCCC |
| | | ATTCGACCACCAAGCGAAACATCGCATCGAGCGAGCACGTACTCG |
| | | GATGGAAGCCGGTCTTGTCGATCAGGATGATCTGGACGAAGAGCA |
| | | TCAGGGGCTCGCGCCAGCCGAACTGTTCGCCAGGCTCAAGGCGAG |
| | | CATGCCCGACGGCGAGGATCTCGTCGTGACCCATGGCGATGCCTG |
| | | CTTGCCGAATATCATGGTGGAAAATGGCCGCTTTTCTGGATTCATC |
| | | GACTGTGGCCGGCTGGGTGTGGCGGACCGCTATCAGGACATAGCG |
| | | TTGGCTACCCGTGATATTGCTGAAGAGCTTGGCGGCGAATGGGCT |
| | | GACCGCTTCCTCGTGCTTTACGGTATCGCCGCTCCCGATTCGCAGC |
| | | GCATCGCCTTCTATCGCCTTCTTGACGAGTTCTTCTGAGCGGGACT |
| | | CTGGGGTTCGAAATGACCGACCAAGCGACGCCCAACCTGCCATCA |
| | | CGAGATTTCGATTCCACCGCCGCCTTCTATGAAAGGTTGGGCTTCG |
| | | GAATCGTTTTCCGGGACGCCGGCTGGATGATCCTCCAGCGCGGGG |
| | | ATCTCATGCTGGAGTTCTTCGCCCACCCTAGGGGGAGGCTAACTG |
| | | AAACACGGAAGGAGACAATACCGGAAGGAACCCGCGCTATGACG |
| | | GCAATAAAAAGACAGAATAAAACGCACGGTGTTGGGTCGTTTGTT |
| | | CATAAACGCGGGGTTCGGTCCCAGGGCTGGCACTCTGTCGATACC |
| | | CCACCGAGACCCCATTGGGGCCAATACGCCCGCGTTTCTTCCTTTT |
| | | CCCCACCCCACCCCCCAAGTTCGGGTGAAGGCCCAGGGCTCGCAG |
| | | CCAACGTCGGGGCGGCAGGCCCTGCCATAGCCTCAGGTTACTCAT |
| | | ATATACTTTAGATTGATTTAAAACTTCATTTTTAATTTAAAAGGAT |
| | | CTAGGTGAAGATCCTTTTTGATAATCTCATGACCAAAATCCCTTAA |
| | | CGTGAGTTTTCGTTCCACTGAGCGTCAGACCCCGTAGAAAAGATC |
| | | AAAGGATCTTCTTGAGATCCTTTTTTTCTGCGCGTAATCTGCTGCT |
| | | TGCAAACAAAAAAACCACCGCTACCAGCGGTGGTTTGTTTGCCGG |
| | | ATCAAGAGCTACCAACTCTTTTTCCGAAGGTAACTGGCTTCAGCA |
| | | GAGCGCAGATACCAAATACTGTCCTTCTAGTGTAGCCGTAGTTAG |
| | | GCCACCACTTCAAGAACTCTGTAGCACCGCCTACATACCTCGCTCT |
| | | GCTAATCCTGTTACCAGTGGCTGCTGCCAGTGGCGATAAGTCGTG |
| | | TCTTACCGGGTTGGACTCAAGACGATAGTTACCGGATAAGGCGCA |
| | | GCGGTCGGGCTGAACGGGGGGTTCGTGCACACAGCCCAGCTTGGA |
| | | GCGAACGACCTACACCGAACTGAGATACCTACAGCGTGAGCTATG |
| | | AGAAAGCGCCACGCTTCCCGAAGGGAGAAAGGCGGACAGGTATC |
| | | CGGTAAGCGGCAGGGTCGGAACAGGAGAGCGCACGAGGGAGCTT |
| | | CCAGGGGGAAACGCCTGGTATCTTTATAGTCCTGTCGGGTTTCGC |
| | | CACCTCTGACTTGAGCGTCGATTTTTGTGATGCTCGTCAGGGGGGC |
| | | GGAGCCTATGGAAAAACGCCAGCAACGCGGCCTTTTTACGGTTCC |
| | | TGGCCTTTTGCTGGCCGAGGGCCTATTTCCCATGATTCCTTCATAT |
| | | TTGCATATACGATACAAGGCTGTTAGAGAGATAATTAGAATTAAT |
| | | TTGACTGTAAACACAAAGATATTAGTACAAAATACGTGACGTAGA |
| | | AAGTAATAATTTCTTGGGTAGTTTGCAGTTTTAAAATTATGTTTTA |
| | | AAATGGACTATCATATGCTTACCGTAACTTGAAAGTATTTCGATTT |
| | | CTTGGCTTTATATATCTTGTGGAAAGGACGAAACACCGAATTCAT |
| | | AAAGCTTGGGCTAAGGGAGATGATTGGGTAGAAAGTACTATTCTA |
| | | TTCATTTGCCTCCCAGCCTACAGTCGACATAGAGCTCTTAGCAGCA |
| | | CGTAAATATTGGCGTTAAGATTCTAAAATTATCTCCAGTATTAACT |
| | | GTGCTGCTGAAGGTACCATATCTAGATTTTTTTTTTGCTCACATGT |
| | | TCTTTCCTGCGTTATCCCCTGATTCTGTGGATAACCGTATTACCGC |
| | | CATGCAT |
| 22 | Designed molecule | 5′-. . . NNNNN**NNNNN . . .- 3′ |
| 23 | mRNA/tRNA | 5′-. . . NNNNNUNNNNNN . . .- 3′ |
| | Substrate | |
| 24 | sctKuuu_40-57R | CGA ACC CCT GAC ATT TCG |
| 25 | sctKcuu_40-57R | CGA ACC CCT AAC CTT ATG |
| 26 | sctRucu_39-56R | CGA ACC CAT AAT CTT CTG |
| 27 | sctLcaa_51-71R | AGA TTC GAA CTC TTG CAT CTT |
| 28 | scSNR81_35-53R | GTC TGT GTA GTT CCA CTT G |
| 29 | HtKuuu_41-62R | GGA CTT GAA CCC TGG ACC CTC A |
| 30 | HtKcuu_41-62R | GGG CTC GAA CCC ACG ACC CTG G |
| 31 | HtSgcu_47-66R | CGA ACC CAC GCG TGC AGA GC |
| 32 | sctKuuu-U36rH | mG mG mU mU mA dA dA dA dG mC mC mG mA mA mU |
| | | mG mC mU mC |
| 33 | sctKcuu-U36rH | mG mA mU mU mA dA dG dA dG mU mC mA mU mA mU |
| | | mG mC mG mC |
| 34 | sctRucu-U36rH | mG mA mU mU mA dG dA dA dG mU mC mA mG mA mU |
| | | mG mC mG mU |
| 35 | scCUP1K30UA | CTA CAT GAT TAG GCT AGC TAC AAC GAT GGC ATT |
| | A_10-23 | GTT C |
| 36 | scCUP1stop_10-23 | AGT CGA CTT AGG CTA GCT ACA ACG ATT CCC AGA GC |
| 37 | Oligo_dT-18 | TTT TTT TTT TTT TTT TTT |
| 38 | CUP1_50-69F | GCC AAT GTG GTA GCT GCA AA |
| 39 | CUP1_133-152R | TCA GAC TTG TTA CCG CAG GG |
| 40 | mCherry_201-220F | CCC TCA GTT CAT GTA CGG CT |
| 41 | mCherry_302-321R | GCC GTC CTC GAA GTT CAT CA |
| 42 | EGFP_466-485F | AAG CAG AAG AAC GGC ATC AA |
| 43 | EGFP_542-561R | GGG GGT GTT CTG CTG GTA GT |
| 44 | TDH3_151-170F | CAC GGT AGA TAC GCT GGT GA |
| 45 | TDH3_254-273R | GTC AAC GTT GGA AGA ACC CC |
| 46 | hACTB_744-765F | CAC CAT TGG CAA TGA GCG GTT C |
| 47 | hACTB_941-962R | GCC AGG GCA GTG ATC TCC TTC T |
| 48 | Cup1^{K30UAA}mRNA | 5′-UGCCAAΨAAUCAUG-3′ |
| 49 | Cup1^{K30UGA}mRNA | 5′-UGCCAAΨGAUCAUG-3′ |
| 50 | Cup1^{K30UAG}mRNA | 5′-UGCCAAΨAGUCAUG-3′ |
| 51 | PTC^{(K30X)}-gRNA | 5′-CAUGAUAC-stem loop-GUUUGGCA(N₈)AUA-3′ |
| 52 | pre-tRNA^{Lys}(UUU) | 5′-UCGGCUUUΨAagcgyau-3′ |
| 53 | Pre-tRNA^{Lys}(CUU) | 5′-AUGACUCUΨAAUCAUAA-3′ |
| 54 | Pre-tRNA^{Arg}(UCU) | 5′-CUGACUUCΨAAUCAGAA-3′ |
| 55 | Sc-TKu | 5′-AUGCGCUGC-stem loop-GUAAAGCCGA(N₆)ACA-3′ |
| 56 | Sc-TKc | 5′-UUAUGAUGC-stem loop-GUAGAGUCAU(N₆)ACA-3′ |
| 57 | Sc-TR | 5′-UUCUGAUGC-stem loop-GUGAAGUCAG(N₆)ACA-3′ |
| 58 | DNAzyme 10-23 | 5′-TTAGGCTAGCTACAACGATGGY-3′ |
| 59 | mCherryK¹⁴uAA | 5′-CCAUCAUCΨAAGAGUUCA-3′ |
| | mRNA | |
| 60 | mCherryK14uAG | 5′-CCAUCAUCΨAGGAGUUCA-3′ |
| | mRNA | |
| 61 | tRNALys(UUU) | 5′-AGACUUUΨAAUCUGAGG-3′ |
| 62 | tRNALyS(CUU) | 5′-RGACUCUΨAAUCYCAGG-3′ |
| 63 | Hs- | 5′-UGAACUCUAG-stem loop-CUGAUGAUGG(N₆)ACA-3′ |
| | PTC^{(K14x)}_gRNA | |
| 64 | Hs-TKu | 5′-CCUCAGAUUC-stem loop-GAAAAGUCU(N₇)ACA-3′ |
| 65 | Hs-TKc | 5′-CCUGRGAUUC-stem loop-GAAGAGUCU(N₇)ACA-3′ |
| 66 | 2′-O-methyl | 5′-ANNNNN-3′ |
| | RNA chimeric | |
| | oligo | |
| 67 | tRNA generic | 5′-NNNNN-3′ |
| | sequence | |
| 68 | mRNA generic | 5′-CCAAΨAA-3′ |
| | sequence | |
| 69 | PTC(K30X) | 5′-UGCCAA((UAA)/(UAG)/(UGA))UCAUGU-3′ |
| | mRNA | |
| 70 | PTC(K30X) | 5′-UGCCAA((ΨAA)/(ΨAG)/(ΨGA))UCAUGU-3′ |
| | pseudouridylated | |
| | mRNA | |
| 71 | PTC(K14X) | 5′-AUCAUC((UAA)/(UAG)/(UGA))GAGUUC-3′ |
| | mRNA | |
| 72 | PTC(K14X) | 5′-AUCAUC((ΨAA)/(ΨAG)/(ΨGA))GAGUUC-3′ |
| | pseudouridylated | |
| | mRNA | |

1. An isolated modified tRNA, comprising a modified anticodon sequence N34N35N36 that comprises a modified uridine at position 36 (N36) of the anticodon sequence.

2. The isolated modified tRNA of claim 1, wherein the modified uridine is pseudouridine (Ψ).

3. The isolated modified tRNA of claim 2, comprising a modified sequence of any one sequence selected from SEQ ID Nos: 1, 2, 3, 4, 5, and 6, wherein the uridine (U) at position 36 (N36) is replaced with pseudouridine (Ψ).

4. The isolated modified tRNA of claim 1, wherein the modified tRNA is tRNA-Lys having a modified anticodon of CUP.

5. The isolated modified tRNA of claim 4, wherein the modified tRNA has a sequence of SEQ ID NO:4, and the uridine at position 36 (N36) is replaced with pseudouridine (Ψ).

6. The isolated modified tRNA of claim 1, wherein the modified tRNA is tRNA-Lys having a modified anticodon of UUΨ.

7. The isolated modified tRNA of claim 6, wherein the modified tRNA has a sequence of SEQ ID NO:5, and the uridine at position 36 (N36) is replaced with pseudouridine (Ψ).

8. The isolated modified tRNA of claim 1, wherein the modified tRNA is tRNA-Arg having a modified anticodon of UCΨ.

9. The isolated modified tRNA of claim 8, wherein the modified tRNA has a sequence of SEQ ID NO:6 and the uridine at position 36 (N36) is replaced with pseudouridine (Ψ).

10. The isolated modified tRNA of claim 1, further comprising one or more modifications in one or more nucleotides of the modified tRNA, wherein the one or more nucleotides comprise a 2′-O-methyl modified sugar moiety, a 2′-O-methoxyethyl modified sugar moiety, a phosphorothioate internucleoside linkage, or a phosphodiester internucleoside linkage, or a combination thereof.

11. The isolated modified tRNA of claim 10, wherein the one or more further modifications are at positions other than position 36 (N36).

12. A method of treating a premature translation termination codon N1N2N3 (PTC)-related disease by nonsense suppression in a subject, the method comprising the steps of:
a) modifying a PTC in an mRNA in a cell of the subject by converting a uridine (U) residue in the PTC into a pseudouridine (Ψ) to create a modified mRNA, wherein the PTC results in the PTC-related disease, wherein the uridine is at position 1 (N1) in the PTC; and
b) providing a modified tRNA in the cell, wherein the modified tRNA comprises a modified uridine at position N36 of an anticodon sequence (N34N35N36);
wherein the modified uridine in the modified tRNA is capable of paring with the pseudouridine (Ψ) in the modified mRNA to attach an amino acid to the translation of the modified mRNA and produce a full length translation product from the modified mRNA.

13. The method of claim 12, wherein the disease is one of Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, or cancer.

14. The method of claim 12, wherein the modified uridine in the modified tRNA is a pseudouridine (Ψ).

15. The method of claim 14, wherein the PTC is UAG and wherein the modified tRNA is tRNA-Lys, having an anticodon sequence of CUΨ.

16. The method of claim 14, wherein the PTC is UAA and wherein the modified tRNA is tRNA-Lys having an anticodon sequence of UUΨ.

17. The method of claim 14, wherein the PTC is UGA and wherein the modified tRNA is tRNA-Arg having an anticodon sequence of UCΨ.

18. The method of claim 12, wherein the step of providing a modified tRNA in the cell comprises the substep of introducing a tRNA targeting nucleic acid molecule for pseudouridylation into the cell, wherein the tRNA targeting nucleic acid molecule comprises a guide region capable of forming a partially double stranded nucleic acid complex with a tRNA in the cell, wherein the partially double stranded nucleic acid complex is capable of engaging a mammalian pseudouridylation enzyme, wherein the guide region assists in positioning the uridine in the tRNA in the partially double stranded nucleic acid complex for it to be converted to a pseudouridine by the mammalian pseudouridylation enzyme.

19. The method of claim 12, wherein the step of modifying a PTC comprises the substep of introducing a mRNA targeting nucleic acid molecule for pseudouridylation into the cell, wherein the mRNA targeting nucleic acid molecule comprises a guide region capable of forming a partially double stranded nucleic acid complex with the mRNA, wherein the partially double stranded nucleic acid complex is capable of engaging a mammalian pseudouridylation enzyme, wherein the guide region assists in positioning the uridine in the PTC in the partially double stranded nucleic acid complex for it to be converted to a pseudouridine by the mammalian pseudouridylation enzyme.

20. The method of claim 12, wherein the step of providing a modified tRNA in the cell comprises the substep of introducing into the cell the modified tRNA.

21. The method of claim 20, wherein the modified tRNA is an artificially synthetic tRNA.

22. The method of claim 12, wherein the modified tRNA further comprises one or more modifications in one or more nucleotides of the modified tRNA, wherein the one or more nucleotides comprise a 2′-O-methyl modified sugar moiety, a 2′-O-methoxyethyl modified sugar moiety, a phosphorothioate internucleoside linkage, or a phosphodiester internucleoside linkage, or a combination thereof.

23. The method of claim 22, wherein the one or more further modifications are at positions other than position 36 (N36).

24. The method of claim 12, wherein the modified tRNA comprises the sequence of SEQ ID NO:13, 14 or 15.

25. The method of claim 12, wherein the modified tRNA comprises the sequence of SEQ ID NO:16, 17 or 18.

26. A method of reading through a premature translation termination codon N1N2N3 (PTC) in an mRNA comprising:
a) modifying the PTC in the mRNA in a cell by converting a uridine (U) residue in the PTC into a pseudouridine (Ψ) to create a modified mRNA, wherein the uridine is at position 1 (N1) in the PTC; and
b) providing a modified tRNA in the cell, wherein the modified tRNA comprises a modified uridine at the last position (position 36 or N36) of an anticodon sequence (N34N35N36) of the modified tRNA;
wherein the modified uridine in the modified tRNA is capable of paring with the pseudouridine (Ψ) in the modified mRNA to incorporate an amino acid at the modified PTC and produce a full length translation product from the modified mRNA.

27. The method of claim 26, wherein the PTC is UAG and wherein the modified tRNA is tRNA-Lyc, having an anticodon sequence of CUΨ.

28. The method of claim 26, wherein the PTC is UAA and wherein the modified tRNA is tRNA-Lys having an anticodon sequence of UUΨ.

29. The method of claim 26, wherein the PTC is UGA and wherein the modified tRNA is tRNA-Arg having an anticodon sequence of UCΨ.

30. The method of claim 26, wherein the step of modifying a PTC comprises the substep of introducing a mRNA targeting nucleic acid molecule for pseudouridylation into the cell, wherein the mRNA targeting nucleic acid molecule comprises a guide region capable of forming a partially double stranded nucleic acid complex with the mRNA, wherein the partially double stranded nucleic acid complex is capable of engaging a mammalian pseudouridylation enzyme, wherein the guide region assists in positioning the uridine in the PTC in the partially double stranded nucleic acid complex for it to be converted to a pseudouridine by the mammalian pseudouridylation enzyme.

31. The method of claim 26, wherein the step of providing a modified tRNA in the cell comprises the substep of introducing a tRNA targeting nucleic acid molecule for pseudouridylation into the cell, wherein the tRNA targeting nucleic acid molecule comprises a guide region capable of forming a partially double stranded nucleic acid complex with a tRNA in the cell, wherein the partially double stranded nucleic acid complex is capable of engaging a mammalian pseudouridylation enzyme, wherein the guide region assists in positioning the uridine in the tRNA in the partially double stranded nucleic acid complex for it to be converted to a pseudouridine by the mammalian pseudouridylation enzyme.

32. The method of claim 26, wherein the step of providing a modified tRNA in the cell comprises the substep of introducing into the cell the modified tRNA.

33. The method of claim 32, wherein the modified tRNA is an artificially synthetic tRNA.

34. The method of claim 32, wherein the modified tRNA further comprises one or more modifications in one or more nucleotides of the modified tRNA, wherein the one or more nucleotides comprise a 2′-O-methyl modified sugar moiety, a 2′-O-methoxyethyl modified sugar moiety, a phosphorothioate internucleoside linkage, or a phosphodiester internucleoside linkage, or a combination thereof.

35. The method of claim 34, wherein the one or more further modifications are at positions other than position 36 (N36).

36. A mRNA/tRNA complex, comprising:
a modified mRNA comprising a modified premature translation termination codon N1N2N3 (PTC), wherein the uridine at position N1 of the PTC has been converted to a pseudouridine (Ψ); and
a modified tRNA, wherein the tRNA comprises a modified uridine at position N36 of the anticodon sequence N34N35N36 of the tRNA,
wherein the pseudouridine (Ψ) in the modified mRNA pairs with the modified uridine in the anticodon sequence of the modified tRNA.

37. The mRNA/tRNA complex of claim 36, wherein the modified uridine in the modified tRNA is pseudouridine (Ψ).

38. The mRNA/tRNA complex of claim 36, wherein the modified PTC is ΨAG and wherein the modified tRNA is tRNA-Lys having a modified anticodon of CUΨ.

39. The mRNA/tRNA complex of claim 36, wherein the modified PTC is ΨAA and wherein the modified tRNA is tRNA-Lys having a modified anticodon of UUΨ.

40. The mRNA/tRNA complex of claim 36, wherein the modified PTC is ΨGA and wherein the modified tRNA is tRNA-Arg having a modified anticodon of UCΨ.

41. The mRNA/tRNA complex of claim 36, the modified tRNA further comprises one or more modifications in one or more nucleotides of the modified tRNA, wherein the one or more nucleotides comprise a 2′-O-methyl modified sugar moiety, a 2′-O-methoxyethyl modified sugar moiety, a phosphorothioate internucleoside linkage, or a phosphodiester internucleoside linkage, or a combination thereof.

42. The mRNA/tRNA complex of claim 41, wherein the one or more further modifications are at positions other than position 36 (N36).

43. A kit for treating a PTC-related disease by nonsense suppression, the kit comprising:
(1) a modified tRNA comprising a modified uridine at position 36 (N36) of an anticodon sequence N34N35N36 of the tRNA;
(2) a nucleic acid molecule for pseudouridylation of a target uridine in a target mRNA in a cell, wherein the nucleic acid molecule comprises a guide region capable of forming a partially double stranded nucleic acid complex with the target mRNA comprising the target uridine, wherein the partially double stranded nucleic acid complex is capable of engaging a mammalian pseudouridylation enzyme, wherein the guide region assists in positioning the target uridine in the partially double stranded nucleic acid complex for it to be converted to a pseudouridine by the mammalian pseudouridylation enzyme, wherein the target uridine is a uridine in a PTC; and
(3) instructions for using the kit.

44. The kit of claim 43, wherein the modified uridine in the modified tRNA is selected from the group consisting of pseudouridine (Ψ), 2′-O-methyl uridine, 2′-O-methoxyethyl uridine, uridine with a phosphorothioate internucleoside linkage and uridine with a phosphodiester internucleoside linkage.

45. A kit for treating a PTC-related disease by nonsense suppression, the kit comprising:
(1) a tRNA targeting nucleic acid molecule for pseudouridylation of a target uridine in a target tRNA, wherein the tRNA targeting nucleic acid molecule comprises a guide region capable of forming a partially double stranded nucleic acid complex with the target tRNA in a cell, wherein the partially double stranded nucleic acid complex is capable of engaging a mammalian pseudouridylation enzyme, wherein the guide region assists in positioning the uridine in the tRNA in the partially double stranded nucleic acid complex for it to be converted to a pseudouridine by the mammalian pseudouridylation enzyme;
(2) a nucleic acid molecule for pseudouridylation of a target uridine in a target mRNA in a cell, wherein the nucleic acid molecule comprises a guide region capable of forming a partially double stranded nucleic acid complex with the target mRNA comprising the target uridine, wherein the partially double stranded nucleic acid complex is capable of engaging a mammalian pseudouridylation enzyme, wherein the guide region assists in positioning the target uridine in the partially double stranded nucleic acid complex for it to be converted to a pseudouridine by the mammalian pseudouridylation enzyme, wherein the target uridine is a uridine in a PTC; and
(3) instructions for using the kit.

46. A method for expanding a genetic codon, the method comprising the step of
a) modifying an original genetic codon (N1N2N3) that contains one or more uridines (U) in a mRNA in a cell by converting the one or more U residues in the original genetic codon into one or more pseudouridines (Ψ) to create a modified mRNA with a modified genetic codon; and
b) providing a modified tRNA in the cell, wherein the modified tRNA comprises an anticodon that
(1) contains one or more pseudouridines (Ψ) that pair with the one or more pseudouridines (Ψ) in the modified genetic codon, and
(2) complements to the modified genetic codon at positions other than the one or more pseudouridines (Ψ),
wherein the modified tRNA pairs with the modified genetic codon in the mRNA and incorporates an amino acid that is different from the amino acid encoded by the original genetic codon into a peptide encoded by the mRNA.
